(19) United States
(12) Patent Application Publication
(10) Pub. No.: US 2024/0002911 A1
(43) Pub. Date: Jan. 4, 2024

(54) Methods Of Signal Amplification

(71) Applicant: Phitonex, Inc., Durham, NC (US)

(72) Inventors: Craig LaBODA, Raleigh, NC (US); Michael STADNISKY, Bend, OR (US); Nicholas PINKIN, Durham, NC (US); Sean BURROWS, Durham, NC (US)

(21) Appl. No.: 18/254,141

(22) PCT Filed: Dec. 9, 2021
(86) PCT No.: PCT/US2021/062552

§ 371 Date: May 23, 2023

Related U.S. Application Data
(60) Provisional application No. 63/237,929, filed on Aug. 27, 2021.
(60) Provisional application No. 63/237,922, filed on Aug. 27, 2021.
(60) Provisional application No. 63/123,770, filed on Dec. 10, 2020.

(51) Int. Cl. C12Q 1/6818; C12Q 1/6841; C12Q 1/6874

(52) U.S. Cl. CPC C12Q 1/6818; C12Q 1/6804; C12Q 1/6874; C12Q 1/6841

(57) ABSTRACT

Provided herein are nucleic acid nanostructure-based compositions that enable amplification of detectable signals and methods that enable tunable, well-controlled, and quantitative amplification of detectable signals from labeled target molecules.

### CROSS-REFERENCE TO RELATED APPLICATIONS

This application is a 35 U.S.C. 371 National Phase of PCT/US2021/062552, filed Dec. 9, 2021, which claims the benefit under 35 U.S.C. § 119(e) of U.S. Provisional Application Nos. 63/237,929 filed Aug. 27, 2021; 63/237,922 filed Aug. 27, 2021 and 63/123,770 filed Dec. 10, 2020. The entire contents of the aforementioned applications are incorporated by reference herein.

### BACKGROUND

Fluorescence-based detection is a ubiquitous tool used throughout the life sciences to observe, identify, and differentiate between various molecular targets of interest, including, but not limited to, antigens on the surface of or within a cell, proteins, antibodies, exosomes, and oligonucleotides. In certain instances, the target molecules or particles may be present at very low concentrations or densities, making them particularly difficult to detect. To circumvent this, researchers typically use very bright fluorophores to observe weakly expressed targets; however, even the brightest fluorophores available with molar extinction coefficients exceeding 1.5 million still often cannot provide enough signal to detect something as rare as a single molecule. In such cases, the fluorescence signal used to detect the target must be amplified by increasing the number of fluorophores that are bound to the target (e.g. through the use of secondary antibodies) rather than by simply choosing a brighter fluorophore. Fluorescence amplification can raise the emission above the threshold of detection, allowing researchers to observe very rare events.

Current methods of fluorescence amplification, however, suffer from important limitations. For example, they are severely limited in their degree of multiplexing, i.e., the number of targets that can be simultaneously observed within a single sample. Current methods typically provide researchers with only a handful of different fluorescent labels to use, which limits the depth or the complexity of the experiment. In addition, many of the conventional amplification methods lack specificity, resulting in the amplification of multiple targets rather than a single, well-defined target. This forces researchers to choose between either performing a low-plex experiment, and amplifying a single target, or a low-resolution experiment, amplifying all targets. Furthermore, current methods of amplification lack well-specified, tunable control over the amplification factor and instead produce a distribution of amplification factors across the various targets, resulting in a high degree of variability and limiting their utility in taking quantitative measurements.

The most common method of detecting cellular markers is through the use of fluorescently labeled primary antibodies. Primary antibodies bind to specific cell markers, providing a direct method of detection once attached to the target molecule. Secondary antibodies, on the other hand, target and bind to primary antibodies based on the host species and isotype of the primary antibody (“Introduction to Secondary Antibodies” Thermo Fisher Scientific, 9 Sep. 2020, at the world wide web at www.thermofisher.com/us/en/home/life-science/antibodies/antibodies-learning-center/antibodies-resource-library/antibody-methods/introduction-secondary-antibodies.html). By labeling secondary antibodies with fluorescent tags, they can be added to a cell sample already containing primary antibodies to indirectly detect the marker to which the primary antibody is bound. The workflow for this indirect detection requires an additional staining step; however, the primary benefit of using secondary antibodies is that multiple secondary antibodies can bind to a single primary antibody, thereby providing an amplified fluorescent signal over that obtained with the primary antibody. Furthermore, the secondary antibodies can also be labeled with other molecules, such as biotin, which when used in conjunction with streptavidin can result in large networks of secondary antibodies, further increasing the amplified fluorescent signal.

One major limitation of amplification via secondary antibodies is that they indiscriminately bind to primary antibodies that share the same host species and isotype. Oftentimes in cell labeling applications, a cell sample will be stained with multiple primary antibodies that each target a different marker but share the same isotype and underlying host species. In such cases, it is impossible to selectively choose which target is amplified, and instead, all primary antibodies of the same isotype/species are amplified when the same fluorescently labeled secondary antibody is added to the sample.

To enable multiplexed detection with secondary antibody amplification, researchers must ensure that the primary antibodies they choose differ in species and/or isotype such that secondary antibodies that each target a different species/isotype, and each carry a different fluorescent tag, are added to the sample to produce an amplified fluorescent signal of a different color for each category of isotype/species. Although this provides some degree of multiplexing, the number of markers that researchers want to observe vastly outnumbers the available isotypes and host species combinations from which to choose, and thus the degree of multiplexing is severely limited. Furthermore, certain primary antibodies may only be available in a specific isotype/species format, making it difficult to design an experiment that allows for specific, targeted amplification of certain subsets of markers.

An alternative approach to amplifying fluorescent signals is to stain cells with antibodies that target the fluorophores, such as phycoerythrin (PE) or Allophycocyanin (APC), rather than the primary antibodies themselves (“Anti-Dye Antibodies” Thermo Fisher Scientific, 9 Sep. 2020, at the world wide web at www.thermofisher.com/us/en/home/life-science/antibodies/primary-antibodies/epitope-tag-antibodies/anti-dye-antibodies.html). These are known as anti-dye antibodies. In this workflow, the user stains cells with fluorescently labeled primary antibodies and then subsequently adds anti-dye antibodies that target and bind to the dyes attached to the primary antibodies. The anti-dye antibody can also be biotinylated, which then allows the user to add streptavidin and biotinylated fluorophores to bind additional fluorophores to the streptavidin labeled anti-dye antibody. In cases where the anti-dye antibody does not quench the target dye, these additional fluorophores amplify the original base signal provided by the primary fluorophore. In cases where the anti-dye antibody does quench the target dye, the new fluorophores can be used to convert the signal over to a brighter, alternative fluorescent dye.

Anti-dye antibodies, however, suffer from a variety of drawbacks as well. First, they are only available for a subset of fluorophores and tandems, which inherently limits the degree of multiplexing that can be achieved. Their use also places constraints on the experimental design and/or instrumentation that can be utilized. Users are forced to design their experiments based around the subset of fluorophores available for amplification, e.g., PE or APC, many of which are known to suffer from fluorescence performance limitations such as cross-laser excitation or spectral spillover. This not only complicates the experimental design but also makes the downstream data analysis difficult.

While the above outlined amplification methods rely on the use of indirect antibodies, branched DNA amplification uses fluorescently tagged oligonucleotides to recognize and enhance the fluorescence signal associated with specific targets (Player A N, Shen L P, Kenny D, Antao V P, Kolberg J A. Single-copy gene detection using branched DNA (bDNA) in situ hybridization. J Histochem Cytochem. 2001; 49(5):603-612.). The general concept begins with the fixation and permeabilization of the cell samples. Custom pairs of oligonucleotides designed to hybridize to specific mRNA and/or DNA targets are then added to the sample. When these primary probe pairs hybridize in adjacent locations, they enable the binding of a long oligonucleotide, called the preamplifier, which behaves as the trunk of the branched amplification network. Shorter amplifier strands are then added, hybridizing to the pre-amplifier strand and providing branches for the binding of many small, fluorescently labeled probe oligos. Multiple probe oligos can bind to each preamplifier, creating one large fluorescent network that amplifies the fluorescence signal for detecting the underlying target mRNA or DNA sequence. This method of amplification has been commercialized by Thermo Fisher Scientific using a product called the PRIMEFLOW RNA Assay (“RNA Detection by Flow Cytometry” Thermo Fisher Scientific, 9 Sep. 2020, at the world wide web at www.thermofisher.com/us/en/home/life-science/cell-analysis/flow-cytometry/flow-cytometry-assays-reagents/rna-detection-flow-cytometry.html). A variety of other in-situ hybridization (ISH) amplification methods that operate in a similar manner also exist, e.g., RNASCOPE (available from, for example, Bio-Techne, Minneapolis, MN, USA).

Branched DNA amplification offers very bright signals with high specificity; however, the degree of multiplexing is greatly limited. First, branched DNA amplification can only be used to target mRNA or DNA sequences rather than a variety of molecular species. Probe oligos are typically labeled with a single fluorophore, practically allowing for only a single color to be detected per laser line (unless fluorophores with unusually large Stokes shifts are utilized). Most other multiplexed fluorescence applications leverage tandems, i.e., Forster resonance energy transfer pairs, to observe more than one color per laser line, but the lack of small, structured, non-protein based tandems that can be used in this application places upper bounds on its multiplexing capabilities.

### SUMMARY

Provided for herein are nucleic acid nanostructure complexes (also referred to herein as “nanostructure complexes”) comprising a primary nucleic acid nanostructure (also referred to herein as “primary nanostructure”) linked to one or more secondary nucleic acid nanostructures (also referred to herein as “secondary nanostructures”). In certain embodiments, the primary nanostructure is linked to a specificity determining molecule. In certain embodiments, the primary nanostructure is linked to the specificity determining molecule and/or secondary nanostructures via a nucleic acid linker, such as wherein the nucleic acid linker is a hybridized at least partially double-stranded linker. In certain embodiments, the primary nanostructure is linked to the specificity determining molecule via a biotin/biotin-binding protein complex and linked to the secondary nanostructures via a nucleic acid linker. In certain embodiments, the nanostructure complex comprises the general structure: P(-L-S)n, wherein P is the primary nanostructure, L is a hybridized at least partially double-stranded nucleic acid linker, S is one or more adjacently linked secondary nanostructures, and n is an integer greater than zero, and wherein: (i) the primary nanostructure comprises n number of partially single-stranded nucleic acid linker extensions, (ii) there are n number of secondary nanostructures comprising an at least partially single-stranded nucleic acid linker extension, and (iii) at least a portion of the nucleic acid sequence of the single-stranded region of the n number of primary nanostructure nucleic acid linker extensions is at least partially complementary to at least a portion of the nucleic acid sequence of the single-stranded region of one of the secondary nucleic acid linker extensions sufficient to form a hybridized at least partially double-stranded linker between the primary nanostructure and each of the secondary nanostructures, thus linking the primary nanostructure to n number of secondary nanostructures.

In certain embodiments, a primary nanostructure comprises one or a combination of fluorophore moieties that give the primary nanostructure a spectral profile and is linked to n number of secondary nanostructures having the same spectral profile, wherein the secondary nanostructures with the same spectral profile amplify the fluorescence signal of the nanostructure complex in comparison to the fluorescence signal of the primary nanostructure alone. In certain embodiments, the secondary nanostructures having the same spectral profile as the primary nanostructure also each comprise the same number of fluorophore moieties as the primary nanostructure and amplify the fluorescence signal of the nanostructure complex by a factor of about n+0.5, n+0.6, n+0.7, n+0.8, n+0.9 or n+1 times the amount of the fluorescence signal of the primary nanostructure alone.

In certain embodiments, a primary nanostructure comprises a unique identifying sequence and is linked to n number of secondary nanostructures comprising the same unique identifying sequence, wherein the secondary nanostructures with the same unique identifying sequence amplify the sequencing signal of the nanostructure complex in comparison to the sequencing signal of primary nanostructure alone. In certain embodiments, the secondary nanostructures also each comprise the same number of unique identifying sequences as the primary nanostructure and amplify the sequencing signal of the nanostructure complex by a factor of about n+0.5, n+0.6, n+0.7, n+0.8, n+0.9 or n+1 times the amount of the sequencing signal of the primary nanostructure alone.

In certain embodiments, a primary nanostructure comprises x number of a fluorophore moiety or a combination of fluorophore moieties and is linked to y number of secondary nanostructures each comprising z number of the same fluorophore moiety or combination of fluorophore moieties as the primary nanostructure, wherein z can be independently determined for each secondary nanostructure and the sum of z from the secondary nanostructures is zₜₒₜₐₗ and the fluorescent signal of the nanostructure complex compared to the fluorescent signal of the primary nanostructure alone is amplified by a factor of about (x+zₜₒₜₐₗ)/x. In certain embodiments, z is the same for each secondary nanostructure and zₜₒₜₐₗ=y*z.

In certain embodiments, a nanostructure complex comprises a primary nucleic acid nanostructure adjacently linked to one or more proximal secondary nucleic acid nanostructures, wherein at least one of the proximal secondary nanostructure is further linked to another secondary nanostructure, optionally, wherein the primary nanostructure and the one or more proximal secondary nanostructures and/or the one or more proximal secondary nanostructures and the another secondary nanostructure linked to the proximal secondary nanostructure is linked via a hybridized at least partially double-stranded nucleic acid linker. In certain embodiments, the nanostructure complex comprises the general formula: P-L₁-S_{P}-L₂-(Sₓ-L_{y})ₙ-Z, wherein P is the primary nanostructure, L₁ is a linker linking the primary nanostructure to a secondary nanostructure, S_{P} (proximal secondary nanostructure) is a secondary nanostructure adjacently linked to the primary nanostructure, L₂ is a linker linking S_{P} to another secondary nanostructure, n is zero or a positive integer, (Sₓ-L_{y}) comprises a secondary nanostructure Sₓ and linker L_{y} linking Sₓ to an additional secondary nanostructure; and Z is an additional one or more secondary nanostructures. In certain embodiments, Z is a terminal secondary nanostructure S_{T}.

Provided for herein are nanostructure complexes comprising a nucleic acid scaffold to which is attached at least one primary nucleic acid nanostructure, wherein the primary nanostructure comprises an at least partially single-stranded nucleic acid linker extension that is hybridized to at least a portion of sequence of the nucleic acid scaffold.

Provided for herein are multiplexed compositions comprising two or more different primary nanostructures, at least one of which is part of a nanostructure complex according to this disclosure.

Provided for herein are methods of labeling a target molecule, the method comprising binding a nucleic acid nanostructure complex according to this disclosure to the target molecule. Further provided for herein are multiplex methods of labeling one or more target molecules comprising binding two or more nanostructure complexes according to this disclosure or at least one nanostructure complex according to this disclosure and at least one primary nanostructure to the one or more target molecules according to a method of this disclosure.

Provided for herein is a kit for performing the method of this disclosure, comprising a nanostructure complex of this disclosure, or a component thereof, and/or comprising reagents and/or apparatus for labeling a target molecule according to the method of this disclosure; optionally, wherein the kit further comprises instructions either printed and/or on an electronic storage medium, buffers and/or additional reagents, and/or packaging materials.

### BRIEF DESCRIPTION OF THE DRAWINGS/FIGURES

FIG. 1 shows: (Left) A primary nucleic acid nanostructure is bound to a target of interest and includes a single-stranded DNA (ssDNA) linker extension (a). (Right) A secondary nanostructure containing a complementary ssDNA linker extension, (a′), hybridizes to (a), forming a primary-secondary nanostructure complex with twice the fluorescence signal intensity.

FIGS. 2A and 2B show three-way multiplexing using secondary nucleic acid nanostructures. FIG. 2A: Primary nucleic acid nanostructures 1, 2, and 3 are bound to targets of interest, respectively, and include ssDNA linker extensions (a), (b), and (c). FIG. 2B: Secondary nucleic acid nanostructures 1, 2, and 3 containing the complementary ssDNA linker extensions (a′), (b′), and (c′) hybridize to (a), (b), and (c), respectively, forming three different colors of primary-secondary nanostructure complexes, each providing twice the fluorescence.

FIG. 3 shows fluorescence spectra for three different nucleic acid nanostructures assembled with either a polyT ssDNA extension or a ssDNA extension with a mixture of bases.

FIGS. 4A and 4B show three-way multiplexed primary nucleic acid nanostructure staining with two-way multiplexed secondary nucleic acid nanostructure amplification. FIG. 4A: Primary nucleic acid nanostructures 1, 2, and 3 are bound to targets of interest, respectively. Primary nucleic acid nanostructures 1 and 3 include ssDNA linker extensions (a) and (c). FIG. 4B: Secondary nucleic acid nanostructures containing complementary ssDNA linker extensions only hybridize to Primary nucleic acid nanostructures 1 and 3 because primary nucleic acid nanostructure 2 does not have a ssDNA linker extension for amplification.

FIGS. 5A and 5B show degenerate secondary nucleic acid nanostructure amplification. FIG. 5A: Primary nucleic acid nanostructure 1 is used to stain multiple targets (1 and 3) while primary nucleic acid nanostructure 2 only stains target 2. FIG. 5B: Secondary nucleic acid nanostructure 2 is added to amplify the fluorescence signal from target 2. Secondary nucleic acid nanostructure 1 is added to amplify the fluorescence signals from targets 1 and 3 simultaneously.

FIGS. 6A and 6B show how secondary nucleic acid nanostructures can convert the resultant spectral profile. FIG. 6A: Primary nucleic acid nanostructures 1, 2, and 3, all containing the same ssDNA linker extension (a) are used to stain targets 1, 2, and 3, respectively. FIG. 6B: A single secondary nucleic acid nanostructure of a different spectral profile from any of the primary nucleic acid nanostructures hybridizes with all three primary nucleic acid nanostructures via its ssDNA linker extension (a′), allowing for every target to now be observed with a single, alternative spectral profile.

FIGS. 7A and 7B are illustrations of tunable amplification factors. FIG. 7A: Primary nucleic acid nanostructures 1 and 2 are bound to targets 1 and 2. Primary nucleic acid nanostructure 1 includes two copies of the same ssDNA linker extension (a), while primary nucleic acid nanostructure 2 contains three copies of the same ssDNA linker extension (b). FIG. 7B: Secondary nucleic acid nanostructure 1 and 2 containing the complementary ssDNA linker extensions (a′) and (b′) hybridize to all available ssDNA linker extensions (a) and (b) respectively, amplifying primary nucleic acid nanostructure 1 by a factor of 3-fold and primary nucleic acid nanostructure 2 by a factor of 4-fold.

FIGS. 8A and 8B illustrate tunable amplification via tunable numbers of fluorophores per secondary nucleic acid nanostructure. FIG. 8A: Primary nucleic acid nanostructures 1 and 2 are bound to targets 1 and 2. Primary nucleic acid nanostructure 1 includes two copies of the same ssDNA linker extension (a) while primary nucleic acid nanostructure 2 contains three copies of the same ssDNA linker extension (b). FIG. 8B: Secondary nucleic acid nanostructures 1 and 2 containing the complementary ssDNA linker extensions (a′) and (b′) hybridize to all available ssDNA linker extensions (a) and (b), respectively. Secondary nucleic acid nanostructure 2 carries twice as many fluorophores as secondary nucleic acid nanostructure 1, resulting in greater amplification.

FIGS. 9A and 9B illustrate secondary nucleic acid nanostructure barcoding. FIG. 9A: Primary nucleic acid nanostructures 1 and 2 are bound to targets 1 and 2. Primary nucleic acid nanostructure 1 includes two ssDNA linker extensions, (a) and (b), while primary nucleic acid nanostructure 2 contains two ssDNA linker extensions, (a) and (c). FIG. 9B: Secondary nucleic acid nanostructures 1, 2, and 3, each having a different spectral profile and containing the complementary ssDNA linker extensions (a′), (b′), and (c′), hybridize to the primary nucleic acid nanostructure extensions (a), (b), and (c), respectively, creating a different three-component barcode for targets 1 and 2.

FIGS. 10A and 10B illustrate secondary nanostructure intensity barcoding. FIG. 10A: Primary nucleic acid nanostructures 1 and 2 are bound to targets 1 and 2, each containing a different number of ssDNA linker extensions, but all with the same sequence (a). FIG. 10B: Secondary nucleic acid nanostructure 1 containing the complementary ssDNA linker extension (a′) hybridizes to all extensions, amplifying target 1 by a factor of 2-fold and target 2 by a factor of 4-fold. These different intensities can be used as a barcode to distinguish between the two targets, even though they use the same signal.

FIGS. 11A and 11B illustrate nanostructure polymerization. Secondary nucleic acid nanostructures 1 and 2 are designed to polymerize with one another. Secondary nucleic acid nanostructure 1 contains two ssDNA linker extensions, both of sequence (a′). Secondary nucleic acid nanostructure 2 contains two ssDNA linker extensions, both of sequence (a) (FIG. 11A). By mixing secondary nucleic acid nanostructures 1 and 2 with the primary nucleic acid nanostructure bound to the target, secondary nucleic acid nanostructures 1 and 2 form a polymer that amplifies the signal for detecting the target (FIG. 11B).

FIGS. 12A and 12B illustrate controlled nucleic acid nanostructure polymerization. Four unique secondary nucleic acid nanostructures, 1 through 4, are designed with two ssDNA linker extensions each, all of which are unique (FIG. 12A). When secondary nucleic acid nanostructures 1 through 4 are added to the primary nucleic acid nanostructure bound to the target, they assemble into a 5-nanostructure linear complex (chain) based on the hybridization of their complementary sequences (FIG. 12B).

FIGS. 13A, 13B and 13C illustrate semi-controlled nucleic acid nanostructure polymerization. A nucleic acid nanostructure set consisting of a primary nucleic acid nanostructure, two different secondary nucleic acid nanostructures 1 and 2 that can ultimately polymerize and a terminating nucleic acid nanostructure (FIG. 13A). Certain nucleic acid nanostructures can be mixed together ahead of time at a well-defined stoichiometry since they do not react with one another. This stoichiometry has an impact on the average polymerization lengths (FIG. 13B). Solutions are mixed to create various nanostructure polymers of various lengths (FIG. 13C).

FIG. 14 shows four nucleic acid nanostructures attached to a single coordinating scaffold strand attached to a target, resulting in a 4-fold signal amplification.

FIG. 15 illustrates that nanostructures with different numbers of ssDNA linker extensions can create large, branched, amplifying networks of nucleic acid nanostructures.

FIG. 16 shows histograms of the signal from monomer and dimer nucleic acid nanostructures (NOVAFLUOR Yellow 610, Thermo Fisher Scientific, Waltham, MA) conjugated to an anti-CD4 antibody and used to stain peripheral blood mononuclear cells (PBMCs). The higher signals from the dimers provide evidence that they formed and were able to amplify the signal relative to the monomer control. Chain-1 and Chain-2 refer to the DNA linkages that bind two nucleic acid nanostructures together, and thus form dimers. Chain-1 and Chain-2 are 24 and 32 nucleotides long, respectively.

FIG. 17 shows size exclusion chromatography (SEC) chromatograms of the NOVAFLUOR Yellow 610 monomer and dimer nucleic acid nanostructures monitored at 260 nm. The shorter elution times of the dimers indicate that their structures are larger than the monomer and provide evidence that the dimer formed. Chain-1 and Chain-2 refer to the DNA linkages that bind two nucleic acid nanostructures together, and thus form dimers. Chain-1 and Chain-2 are 24 and 32 nucleotides long, respectively.

FIGS. 18A, 18B and 18C shows example histograms showing the signal from primary and primary+secondary (i.e., dimer) nucleic acid nanostructure-antibody conjugates on PBMCs. Illustrative signal from a dimer after adding a secondary nucleic acid nanostructure, in a separate step, to the primary nucleic acid nanostructure-antibody conjugate (FIG. 18A), illustrative signal from the primary nucleic acid nanostructure-antibody conjugate prior to adding the secondary nucleic acid nanostructure (FIG. 18B), illustrative signal after adding a secondary nucleic acid nanostructure negative control (i.e. no linker extension) to the primary nucleic acid nanostructure-antibody conjugate (FIG. 18C).

FIGS. 19A, 19B and 19C show example data for two-step staining with two spectrally unique nucleic acid nanostructures. For this example, the two nucleic acid nanostructures are NOVAFLUOR Red 710 and NOVAFLUOR Yellow 590. FIG. 19A shows an illustrative spectral profile from primary nucleic acid nanostructure, NOVAFLUOR Yellow 590, before adding the secondary nucleic acid nanostructure, NOVAFLUOR Red 710, that has a different spectral profile. FIG. 19B shows two illustrative unique spectral profiles when both NOVAFLUOR Red 710 and NOVAFLUOR Yellow 590 are present (i.e., after adding secondary nucleic acid nanostructure NOVAFLUOR Red 710 in a separate step). FIG. 19C shows the resultant illustrative spectral profile when adding the secondary negative control NOVAFLUOR Red 710.

FIG. 20 shows a schematic illustration of three fluorescence amplification strategies using streptavidin as a cross-linker.

FIG. 21 shows a demonstration of Strategy 1 from FIG. 20 wherein human PBMCs were stained using an anti-CD4 antibody-biotin conjugate, washed, and then stained with a streptavidin-conjugated nucleic acid nanostructure, in this example, streptavidin-NOVAFLUOR Yellow 610 was used. Streptavidin was labeled with NOVAFLUOR Yellow 610 using amine-reactive chemistry, leaving open all biotin-binding sites. The staining is compared to CD4 staining using a primary antibody directly labeled with NOVAFLUOR Yellow 610.

FIG. 22 shows a demonstration of Strategy 2 from FIG. 20 wherein human PBMCs were stained using an anti-CD4 antibody-biotin conjugate, washed, and then stained with a streptavidin-conjugated nucleic acid nanostructure, in this example, streptavidin-NOVAFLUOR Yellow 610-Biotin was used. Streptavidin was labeled with biotinylated NOVAFLUOR nucleic acid nanostructure at the ratios indicated. The staining is compared to CD4 staining using a primary antibody directly labeled with NOVAFLUOR Yellow 610.

FIG. 23 shows a demonstration of Strategy 3 from FIG. 20 wherein human PBMCs were stained with a complex of anti-CD4 antibody-biotin conjugate, streptavidin, and biotinylated NOVAFLUOR Yellow 610 nucleic acid nanostructure. The staining was compared to CD4 staining using a primary antibody directly labeled with NOVAFLUOR Yellow 610.

FIG. 24 is a demonstration of Strategy 2 from FIG. 20 using nucleic acid nanostructure dimers to further amplify the fluorescence signal. Human PBMCs were stained using an anti-CD4 antibody-biotin conjugate, washed, and then stained with streptavidin-NOVAFLUOR Yellow 610 nucleic acid nanostructure monomer or dimer. Streptavidin was labeled with biotinylated NOVAFLUOR monomer or dimer at a 2:1 ratio. The staining was compared to CD4 staining using a primary antibody directly labeled with NOVAFLUOR Yellow 610. Chain-1 and Chain-2 refer to the DNA linkages that bind two nucleic acid nanostructures together, and thus form dimers. Chain-1 and Chain-2 were 24 and 32 nucleotides long, respectively.

FIGS. 25A, 25B and 25C show histograms of the signal from monomer and dimer NOVAFLUOR Ultraviolet CD4 stained PBMCs. FIGS. 25A, 25B, and 25C depict NOVAFLUOR Ultraviolet 430, NOVAFLUOR Ultraviolet 445, and NOVAFLUOR Ultraviolet 755, respectively. The higher signals from the dimers provide evidence that they formed and were able to amplify the signal relative to the monomer control.

### DETAILED DESCRIPTION

### Definitions

It is to be noted that the term “a” or “an” entity refers to one or more of that entity; for example, “a linker,” is understood to represent one or more linkers. As such, the terms “a” (or “an”), “one or more,” and “at least one” can be used interchangeably herein.

Furthermore, “and/or” where used herein is to be taken as specific disclosure of each of the specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever aspects are described herein with the language “comprising” or “comprises” otherwise analogous aspects described in terms of “consisting of,” “consists of,” “consisting essentially of,” and/or “consists essentially of,” and the like are also provided.

Unless defined otherwise, technical and scientific terms used herein have the same meaning as commonly understood by one of ordinary skill in the art to which this disclosure is related.

Numeric ranges are inclusive of the numbers defining the range. Even when not explicitly identified by “and any range in between,” or the like, where a list of values is recited, e.g., 1, 2, 3, or 4, unless otherwise stated, the disclosure specifically includes any range in between the values, inclusive of the end-points, e.g., 1 to 3, 1 to 4, 2 to 4, etc.

The headings provided herein are solely for ease of reference and are not limitations of the various aspects or aspects of the disclosure, which can be had by reference to the specification as a whole.

As used herein, a “linker” is a component of a conjugated molecule whose purpose is to link together other components of the molecule or, when the other components of the conjugated molecule are not linked together, the portion of a component present for the purpose of conjugating to another constituent but that would otherwise not necessarily be present. For example, an antibody would not normally or necessarily have a polynucleotide attached to it, but for the purposes of this disclosure, a polynucleotide can be attached to an antibody to form a linker to link the antibody to another molecule to form a conjugate molecule. Likewise, a nucleic acid nanostructure of this disclosure may not necessarily have a certain at least partially single-stranded extension, but for the purposes of this disclosure, a nucleic acid nanostructure can comprise an at least partially single-stranded linker extension to link the nanostructure to another molecule, such as an antibody, to form a conjugate molecule.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring.”

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-standard amino acids. A polypeptide can be derived from a natural biological source or produced by recombinant technology but is not necessarily translated from a designated nucleic acid sequence. It can be generated in any manner, including by chemical synthesis.

A “protein” as used herein can refer to a single polypeptide, i.e., a single amino acid chain as defined above, but can also refer to two or more polypeptides that are associated, e.g., by disulfide bonds, hydrogen bonds, or hydrophobic interactions, to produce a multimeric protein.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are recombinant polypeptides that have been separated, fractionated, or partially or substantially purified by any suitable technique.

Other polypeptides disclosed herein are fragments, derivatives, analogs, or variants of the foregoing polypeptides, and any combination thereof. The terms “fragment,” “variant,” “derivative” and “analog” when referring to polypeptide subunit or multimeric protein as disclosed herein can include any polypeptide or protein that retains at least some of the activities of the complete polypeptide or protein, but which is structurally different. Fragments of polypeptides include, for example, proteolytic fragments, as well as deletion fragments. Variants include fragments as described above, and also polypeptides with altered amino acid sequences due to amino acid substitutions, deletions, or insertions. Variants can occur spontaneously or be intentionally constructed. Intentionally constructed variants can be produced using art-known mutagenesis techniques. Variant polypeptides can comprise conservative or non-conservative amino acid substitutions, deletions or additions. Derivatives are polypeptides that have been altered so as to exhibit additional features not found on the native polypeptide. Examples include fusion proteins. Derivative polypeptides can also be referred to herein as “polypeptide analogs.” As used herein a “derivative” can refer to a subject polypeptide having one or more amino acids chemically derivatized by reaction of a functional side group. Also included as “derivatives” are those peptides that contain one or more standard or synthetic amino acid derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

As used herein, the term “specificity determining molecule” refers in its broadest sense to a molecule that recognizes a target molecule (target) and associates with it. Specificity determining molecules include binding molecules that can specifically bind to an antigenic determinant, such as an antibody binds an epitope, and also molecules that can bind to receptors, such as receptor ligands (e.g., gastrin-releasing peptide (GRP) and gastrin-releasing peptide receptor (GRPR)). Thus, representative examples of specificity determining molecules include peptides, recombinant, natural, or engineered receptor/ligand proteins, aptamers, tetramers (folded MHC proteins with peptides used for detecting T cell receptors), non-antibody proteins or antibody mimetics, e.g., affilins, affimers, affitins, alphabodies, avimers, fynomers, Kunitz domain peptides, nanoCLAMPS, Designed Ankyrin Repeat Proteins (DARPins), monobodies, anticalins, affibodies, and SOMAmers (further examples are referred to in the Global Bioanalysis Consortium (GBC) and the European Medicines Agency “classification of critical reagents as analyte specific or binding reagents, specifically antibodies; peptides; engineered proteins; antibody, protein and peptide conjugates; reagent drugs; aptamers and anti-drug antibody (ADA) reagents including positive and negative controls (King, L E, et al. Ligand Binding Assay Critical Reagents and Their Stability: Recommendations and Best Practices from the Global Bioanalysis Consortium Harmonization Team. AAPS J. 2014 May; 16(3): 504-515). In certain embodiments, a specificity determining molecule may target genomic material, e.g. DNA or RNA, to perform fluorescence in situ hybridization (FISH) or other biological assays, e.g., on chromatin accessibility or gene expression.

Disclosed herein are certain binding molecules comprising antibodies, or antigen-binding fragments, variants, or derivatives thereof. Unless specifically referring to full-sized antibodies such as naturally-occurring antibodies, the term “binding molecule” encompasses full-sized antibodies including bispecific antibodies (e.g., comprising a first binding domain binding to a first epitope, and a second binding domain binding to a second epitope), as well as antigen-binding fragments, variants, analogs, or derivatives of such antibodies, e.g., naturally-occurring antibody or immunoglobulin molecules or engineered antibody molecules or fragments that bind antigen in a manner similar to antibody molecules.

The terms “antibody” and “immunoglobulin” can be used interchangeably herein. Basic immunoglobulin structures in vertebrate systems are relatively well understood. See, e.g., Harlow et al., Antibodies: A Laboratory Manual, (Cold Spring Harbor Laboratory Press, 2nd ed. 1988). Antibodies or antigen-binding fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

As used herein, the term “chimeric antibody” will be held to mean any antibody wherein the immunoreactive region or site is obtained or derived from a first species and the constant region (which can be intact, partial or modified) is obtained from a second species. In some embodiments the target binding region or site will be from a non-human source (e.g. mouse or primate) and the constant region is human.

The term “bispecific antibody” as used herein refers to an antibody that has binding sites for two different antigens within a single antibody molecule. It will be appreciated that other molecules in addition to the canonical antibody structure can be constructed with two binding specificities. It will further be appreciated that antigen binding by bispecific antibodies can be simultaneous or sequential. Triomas and hybrid hybridomas are two examples of cell lines that can secrete bispecific antibodies. Bispecific antibodies can also be constructed by recombinant means. (Strohlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which the variable domain in either the heavy chain, light chain or both is altered by at least partial replacement of one or more CDRs from an antibody of known specificity and, by partial framework region replacement and sequence changing. Although the CDRs can be derived from an antibody of the same class or even subclass as the antibody from which the framework regions are derived, it is envisaged that the CDRs will be derived from an antibody of different class, e.g., from an antibody from a different species. An engineered antibody in which one or more “donor” CDRs from a non-human antibody of known specificity is grafted into a human heavy or light chain framework region is referred to herein as a “humanized antibody.” In some instances, not all of the CDRs are replaced with the complete CDRs from the donor variable region to transfer the antigen binding capacity of one variable domain to another; instead, minimal amino acids that maintain the activity of the target-binding site are transferred. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

The term “polynucleotide” (also referred to as an “oligonucleotide”) is intended to encompass a singular nucleic acid as well as plural nucleic acids with “nucleic acid” referring to, for example, DNA or RNA or an analog thereof such as comprising a synthetic backbone or base. In certain embodiments, the polynucleotide or nucleic acid is DNA. In other embodiments, a polynucleotide or nucleic acid can be RNA. A nucleic acid or polynucleotide can comprise a conventional phosphodiester bond or a non-conventional bond (e.g., an amide bond, such as found in peptide nucleic acids (PNA)). By “isolated” nucleic acid or polynucleotide is intended a nucleic acid molecule, DNA or RNA, which has been removed from its native environment such as an isolated nucleic acid molecule or construct, e.g., messenger RNA (mRNA) or plasmid DNA (pDNA). For example, a recombinant polynucleotide encoding a polypeptide subunit contained in a vector is considered isolated as disclosed herein. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, a polynucleotide or a nucleic acid can be or can include a regulatory element such as a promoter, ribosome binding site, or a transcription terminator.

A “nucleic acid nanostructure” is an oligonucleotide construction of any size and composed of one or more oligonucleotide strands and can have a tertiary and/or a quaternary structure and be composed of natural and/or synthetic nucleic acid bases. A nucleic acid nanostructure comprised substantially or entirely of DNA is also referred to herein as a DNA nanostructure. In certain embodiments, a nucleic acid nanostructure can include fluorescent moieties of any type, including but not limited to small organic dyes (of all varieties and base structures, e.g. rhodamines, cyanines, oxazines, etc.), naturally occurring fluorophores, phosphorescent molecules, fluorescent proteins, fluorescent polymers, quantum dots or other fluorescent nanoparticles, upconverting particles, lanthanides, and bioluminescent molecules, as well as unique identifying sequences. As used herein, the terms “nucleic acid nanostructure” and “nanostructure” are interchangeable.

As used herein, a “PHITON” (Thermo Fisher Scientific, Waltham, MA) is a nucleic acid nanostructure produced by PHITONEX, Inc. (now a part of Thermo Fisher Scientific), Durham, North Carolina (U.S. Patent Publication No. 2020/0124532, Lebeck, A., Dwyer, C., LaBoda C., Resonator Networks for Improved Label Detection, Computation, Analyte Sensing, and Tunable Random Number Generation; which is incorporated herein in its entirety). PHITONs are fluorescent labels composed of a DNA-based scaffold that precisely arranges fluorophores in order to engineer their interactions and the overall fluorescent properties of the structure. The underlying scaffold presents many unique opportunities for fluorescence amplification. For example, as disclosed herein, the underlying scaffold can be leveraged to programmatically control the interactions between individual PHITONs in order to chain them together for a collectively enhanced fluorescence signal. Examples of the PHITON nucleic acid nanostructure are NOVAFLUOR nucleic acid nanostructures (Thermo Fisher Scientific, Waltham, MA).

As used herein, unless otherwise specified, “complementary base pairing” refers to A/T, A/U, or C/G base pairing and corresponding pairing of synthetic or non-standard nucleotides, e.g., isocytosine/isoguanine (isoC/isoG). To the extent that thymidine (T) is specified as a base in a nucleic acid, for the purposes of simplifying this disclosure, unless otherwise specified, it is understood that uracil (U) is intended if the nucleic acid is RNA.

Unless otherwise specified in a particular context, the terms “conjugated to” and “linked to” are used interchangeably herein.

As used herein, a “conjugate” is a composition having distinct parts, components moieties, constituents, or the like linked together.

As used herein, “cell enrichment” modalities include magnetic or bubble-based enrichment including positive or negative enrichment via metal particles or microbubbles conjugated to specificity determining molecules and microfluidic-based cell enrichment based on size or other characteristics e.g., fluorophore-conjugated specificity determining molecules; or a combination of one or more these methods (generally the concept is enriching either positively or negatively based on cell characteristics like identity, size, granularity, mass, etc.).

“Cell sorting” modalities such as fluorescence-activated cell sorting (FACS) includes the use of fluorophore-conjugated specificity determining molecules to sort/enrich cell population(s) of interest, e.g., for downstream analysis.

As used herein, “immunofluorescent cell labeling” modalities involve the process in which antigens (such as protein antigens) of interest that are expressed in or on a cell can be detected using primary antibodies covalently conjugated to fluorophores (direct detection), a two-step approach with unlabeled primary antibody followed by fluorophore-conjugated secondary antibody (indirect detection), or other variations known to those of skill in the art. Additionally, such methods can include the use of cell membrane or DNA stains. In this manner, one or a multitude of cells from one or more samples, tissues, patients, etc., can be measured via immunofluorescent techniques (flow cytometry, immunofluorescence imaging, etc.) and/or enriched such as via FACS.

As used herein, “genomic analysis” modalities involve the examination of the transcriptome (identity, copy number of mRNA or other RNA species including alternative transcript isoforms and single nucleotide polymorphisms (SNPs), either using whole transcriptome analysis (WTA) or using targeted panels (e.g., examining 100s of selected genes), on a per-cell or per-tissue basis, as well as potentially determining the location of the RNA in combination with its identity); T and or B cell receptor sequencing in which DNA sequencing is performed to examine the receptors of these immune cells; DNA sequencing to examine germline DNA e.g. to detect copy-number variation (CNV) at a single cell level; the use of sequence-tagged antibodies to examine protein/antigen expression through methods such as Cellular Indexing of Transcriptomes and Epitopes by Sequencing (CITE-Seq), TOTALSEQ (BioLegend, San Diego, CA) (“proteogenomics”) or AbSeq (BD Biosciences); assessing DNA accessibility and chromatin e.g. through single cell ATAC-Seq; assessing the extent and targets of gene editing e.g. through single cell CRISPR screens; or a combination of one or more of the methods listed above. In addition to cells in suspension based methods, genomic analysis also includes the addition of location-based data either through assaying genomic material directly e.g., FISH, Multiplexed Error-Robust Fluorescence In Situ Hybridization (MERFISH), spatial transcriptomics or by leveraging a sequence tag to assay the presence and location of proteins and other antigens e.g. through the use of sequence-tagged antibodies. The measurement of either in-solution or location-based assays could include the use of Sanger sequencing, next-generation sequencing, long read sequencing, or in situ sequencing.

As used herein, a “fluorescent label” (also called a fluorophore, fluorescent tag, fluorescent dye, or fluorescent probe) is a molecule that is attached to aid in the detection of a biomolecule such as a protein, antibody, or amino acid. A fluorescent label may be a naturally occurring fluorescent protein (e.g. phycoerythrin, PE), a derivative thereof (e.g. PE-Cy7) including tandem dyes, polymer dyes, single molecule dyes, fluorescent nucleic acids, or scaffold-based fluorescent labels e.g. nucleic acid nanostructure including fluorescent DNA nanostructures such as NOVAFLUOR nucleic acid nanostructures (Thermo Fisher Scientific, Waltham, MA). Examples of NOVAFLUOR nucleic acid nanostructures include, but are not limited to, NOVAFLUOR Ultraviolet 430, NOVAFLUOR Ultraviolet 445, NOVAFLUOR Ultraviolet 755, NOVAFLUOR Blue 510, NOVAFLUOR Blue 530, NOVAFLUOR Blue 555, NOVAFLUOR Blue 585, NOVAFLUOR Blue 610/30S, NOVAFLUOR Blue 610/70S, NOVAFLUOR Blue 660/40S, NOVAFLUOR Blue 660/120S, NOVAFLUOR Yellow 570, NOVAFLUOR Yellow 590, NOVAFLUOR Yellow 610, NOVAFLUOR Yellow 660, NOVAFLUOR Yellow 690, NOVAFLUOR Yellow 700, NOVAFLUOR Yellow 730, NOVAFLUOR Red 660, NOVAFLUOR Red 685, NOVAFLUOR Red 700 and NOVAFLUOR Red 710.

As used herein, a “spectral profile” means a well-defined absorbance and/or emission spectrum of a nanostructure and/or nanostructure complex as a whole, defined by the identity, composition, number, placement, orientations, and/or interactions (e.g. FRET) between the fluorophore moieties attached to the nanostructure and/or nanostructure complex.

As used herein, a “target” molecule refers to an epitope or something that can be targeted genomically, e.g., through DNA complementarity. In certain embodiments, epitopes/antigens can be targeted using a specificity determinant such as an antibody or binding fragment thereof.

Unless otherwise specified, the terms “barcode,” “feature barcode,” and “unique identifying sequence” are used interchangeably and refer to an oligonucleotide sequence that can be used to distinguish between one or multiple species.

### Overview

Current methods of fluorescence amplification offer a limited degree of multiplexing and specificity, making it difficult to study many different low density (or low concentration) targets in a single experiment. Further, there are currently no fluorescence amplification methods that provide both tunable and well-controlled, quantitative amplification (and particularly not across the multiple orders of magnitude needed to detect single molecules). Existing methods result in a distribution of amplification factors across targets, which inevitably introduces variability in the amplified signal.

Provided herein are compositions and methods utilizing nanostructure complexes comprising secondary nucleic acid nanostructures, also referred to herein as “secondary nanostructures,” designed to recognize, bind to, amplify, and/or alter the signal of primary nucleic acid nanostructures, also referred to herein as “primary nanostructures,” of the nanostructure complex. In certain embodiments, the use of secondary nanostructures amplifies the intensity of the signal of a nanostructure complex in comparison to the signal of a primary nanostructure alone. As disclosed in detail herein, in contrast to current workflows, the use of secondary nanostructures provides a much higher limit of multiplexing, greater specificity regarding the target molecules, and very fine control over the amplification factor.

While many exemplary and illustrative embodiments outlined throughout this disclosure may refer to amplifying fluorescence signals, it should be noted that the general concept of controllably increasing the number of nanostructures bound to a specific target also enables amplification of sequence signals, such as provided by unique identifying sequences that are part of the nucleic acid linker extensions between nucleic acid nanostructures or are incorporated into the sequence of a nucleic acid nanostructure itself. For purposes of this disclosure, unless otherwise specified, a nucleic acid nanostructure comprising a nucleic acid linker extension that comprises a unique identifying sequence is considered to comprise the unique identifying sequence. It should be noted that the general concept of controllably increasing the number of nanostructures bound to a specific target to control the number of unique identifying sequences present can also be used to control the number of DNA binding proteins, enzymes, substrates, or other “cargo” to be associated with the nanostructure and/or nanostructure complex including when the nanostructure complex is bound a target.

In certain embodiments, target molecules are first stained (bound) with a primary nanostructure that either is capable of recognizing a target molecule or is attached to, for example, an antibody or other biomolecules that recognize and bind to a target (i.e. specificity determining molecule). In certain embodiments, a primary nanostructure includes an at least partially single-stranded DNA (ssDNA) linker extension off of the edge of the underlying DNA nanostructure (for brevity, unless otherwise stated, a “single-stranded DNA linker extension” refers to both an at least partially ssDNA linker extension and an entirely ssDNA linker extension). This extension allows for the hybridization of a secondary nanostructure which contains a complementary ssDNA sequence. In certain embodiments, the secondary nanostructure is labeled with the same fluorophores or otherwise comprises the same spectral profile, and upon hybridization, the secondary nanostructure amplifies the fluorescence signal of the primary nanostructure as there are now two attached nanostructures of the same spectral profile per target molecule. As explained in detail herein, this concept extends to the addition of additional secondary nanostructures and/or fluorophores and also to the control of the number of unique identifying sequences and amplification of a sequencing signal.

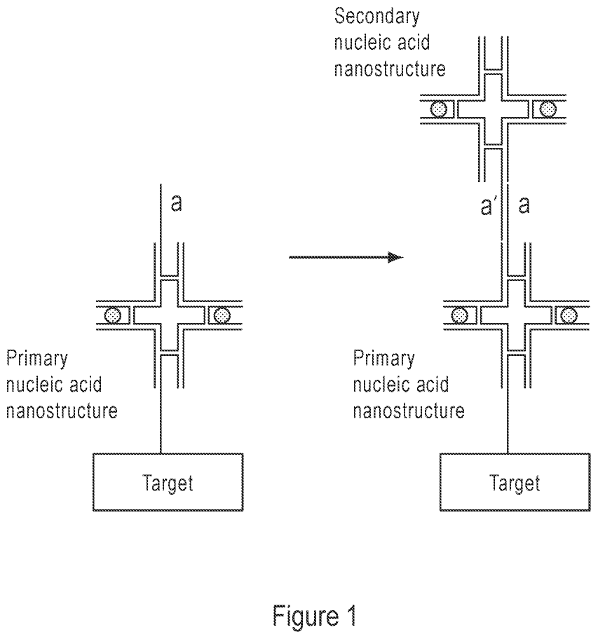

FIG. 1 illustrates an example where a target has been stained with a primary nanostructure comprising an at least partially single-stranded DNA (ssDNA) linker extension (a). A secondary nanostructure comprising a complementary at least partially ssDNA linker extension (a′) is contacted with the primary nanostructure to attach it to the primary nanostructure. The two nanostructures hybridize to one another via their linker extensions, forming a stable primary-secondary nanostructure complex that can theoretically provide twice the fluorescence signal. In certain other embodiments, the primary and secondary nanostructures may be hybridized prior to staining the target, but for the sake of clarity and consistency throughout this disclosure, unless otherwise stated, examples will assume that the target is first stained with the primary nanostructure before attachment of any secondary nanostructures. It is understood, however, that any other order of linking of nanostructure complex components and/or binding to the target are contemplated.

Further, it is understood that although generally the methods outlined throughout this disclosure refer to amplifying fluorescence signals through the use of secondary nanostructures that comprise additional fluorophores with the same spectral profile, in certain embodiments, secondary nanostructures may also comprise fluorophores that alter the spectral profile of the primary nanostructure, be blank, i.e., contain no fluorophores, and/or even contain quenchers that intentionally absorb the fluorescence of the primary nanostructure. In certain embodiments, a blank secondary nanostructure can be used to amplify the number of unique DNA sequences attached to a target such as can be useful in sequencing applications. In certain embodiments, a secondary quenching nanostructure can be used to reduce the fluorescence signal from a specific target, for example in the case where certain fluorescence signals are too bright, and therefore make it difficult to detect other signals in a multiplexed application.

The sequence space provided by use of ssDNA linker extensions enables a high degree of multiplexing for fluorescence amplification applications when using secondary nanostructures. For example, using just 5 bases per ssDNA linker extension, there are 4\hat{ }5 different unique sequences (based on the standard bases A, T, C, and G), or 512 different sets of complementary sequences, each of which can potentially be used for a different color of primary-secondary nanostructure pairs—although, in practice, some of this sequence space should be avoided based on secondary structures and/or the potential for dimer formation between off-target primary-secondary nanostructure pairs. Regardless, the exponential scaling with respect to the length of the ssDNA linker extensions implies that the multiplexing limits of this method vastly outnumber the isotype/species multiplexing options available to secondary antibody-based amplification methods.

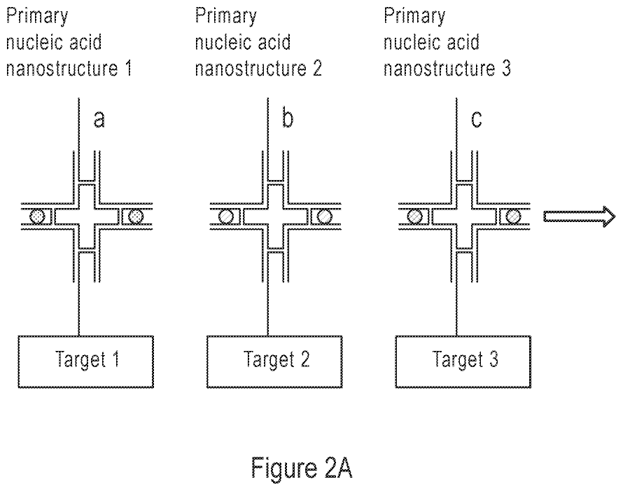

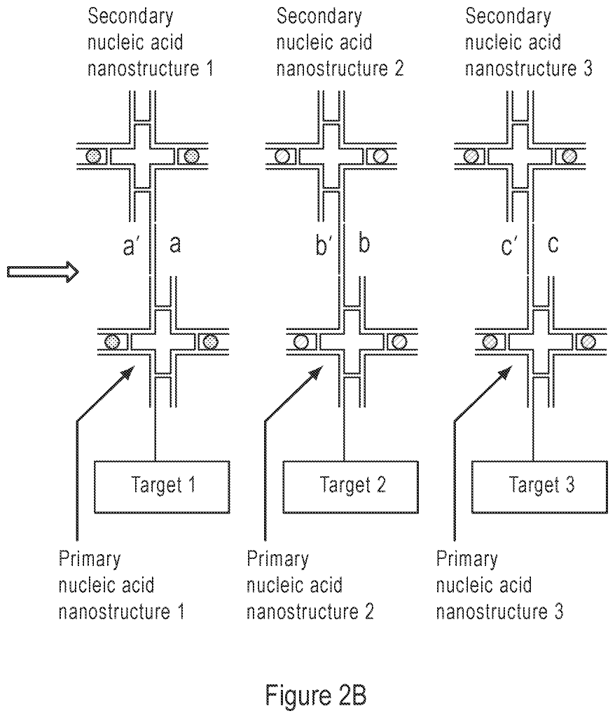

FIGS. 2A-2B demonstrate multiplexed fluorescence amplification via secondary nanostructures. For example, primary nanostructures 1, 2, and 3, each having a different spectral profile, are first used to stain three separate target molecules. These primary nanostructures contain ssDNA linker extensions (a), (b), and (c) respectively. Secondary nanostructures 1, 2, and 3, with complementary ssDNA linker extensions (a′), (b′), and (c′), respectively, are then added, which hybridize to the corresponding primary nanostructures, and amplify the original fluorescence signals. Through this example, one of ordinary skill in the art can understand that numerous additional primary nanostructures, targeting additional target molecules, can each be attached to additional secondary nanostructures to amplify or otherwise modulate their signal, to achieve an unprecedented degree of multiplexing in a sample. In certain embodiments, at least about 2, 3, 4, 5, 6, 7, 8, 9, 10, 15, 20, 25, 30, 40, 50, 75, 100, 200, 300, 400, 500, 600, 700, 800, 900, or 1000 different target molecules can be labeled in such manner. In certain embodiments, between any of about 2, 3, 4, 5, 6, 7, 8, 9, 10, 15, 20, 25, 30, 40, 50, 75, 100, 200, 300, 400, 500, 600, 700, 800, or 900 and any of about 3, 4, 5, 6, 7, 8, 9, 10, 15, 20, 25, 30, 40, 50, 75, 100, 200, 300, 400, 500, 600, 700, 800, 900, or 1000 different target molecules can be labeled in such manner.

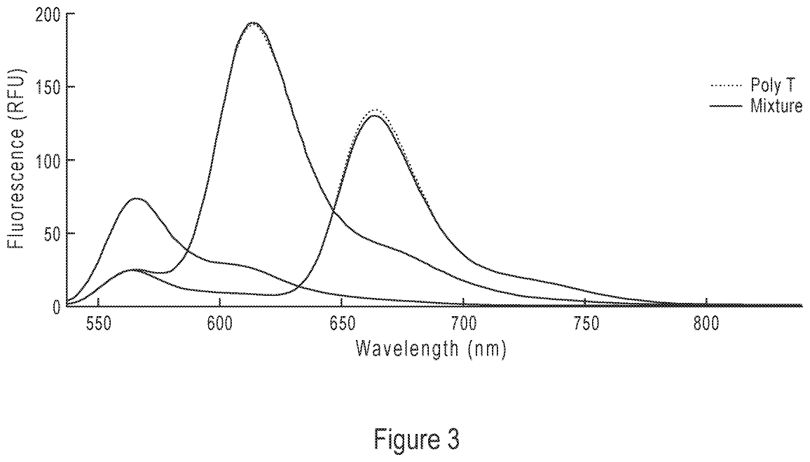

Importantly, it has been observed that the inclusion of different ssDNA linker extensions on a nanostructure has very little impact on both the fluorescence properties of the nanostructure as well as its assembly. FIG. 3 shows the fluorescence spectrum of three different nanostructures, each assembled with two different ssDNA linker extensions. In one case, the linker extension consists of a polyT sequence, while in the other case, the linker extension is a unique sequence containing a mixture of bases. In this example, the fluorescence spectra are nearly identical. Thus, it is contemplated that as long as problematic sequences are avoided, e.g., G-quadruplexes or I-motifs, the ssDNA linker extensions can be swapped out with little effect on downstream applications.

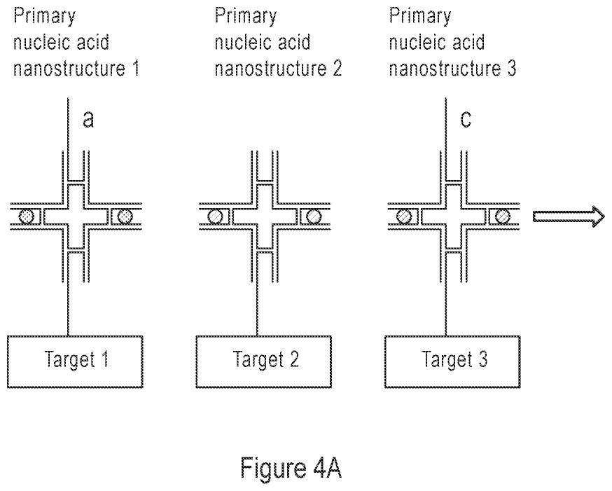

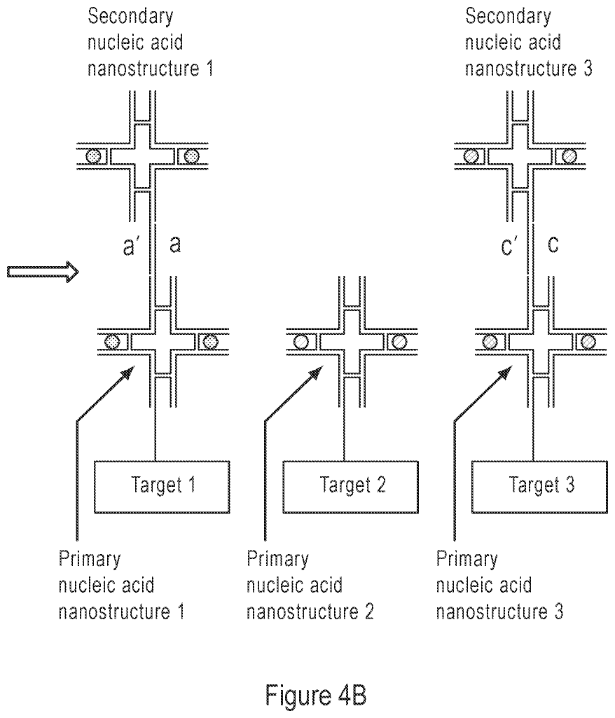

Further, when using primary-secondary nanostructure complexes, researchers can choose which signals are amplified, i.e., the method not only has a high multiplexing upper limit, but it is also very specific. FIGS. 4A-4B show an example in which three separate targets are stained with primary nanostructures 1, 2, and 3, each having a different spectral profile. Unlike the example in FIGS. 2A-2B though, only primary nanostructures 1 and 3 contain ssDNA extensions—(a) and (c), respectively. Primary nanostructure 2 does not have a linker extension to serve as an additional binding region. Thus, even if a mixture of secondary nanostructures that typically target primary nanostructures 1, 2, and 3 are added to the sample, only primary nanostructures 1 and 3 are amplified since a secondary nanostructure cannot bind to primary nanostructure 2.

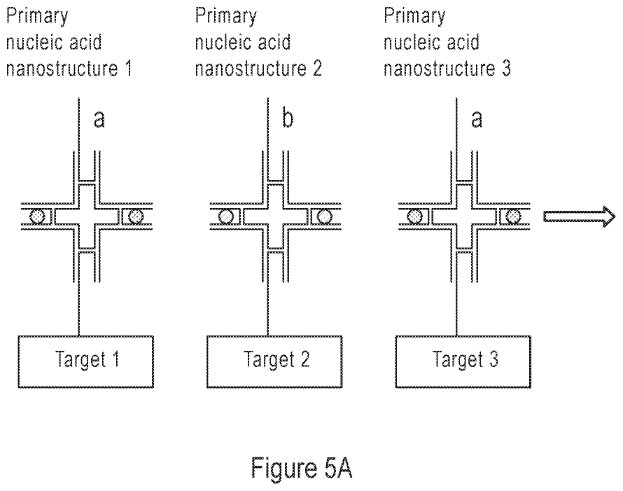

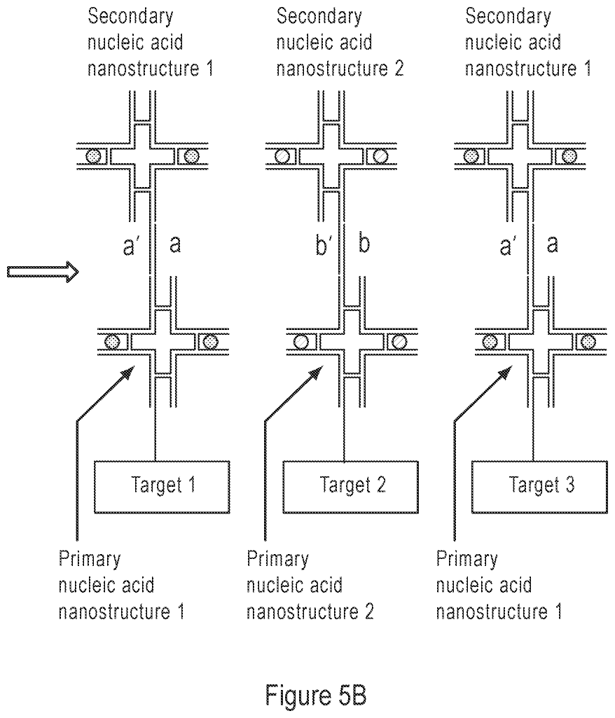

In the examples above, each unique primary nanostructure (in certain embodiments meaning having a different spectral profile and/or in certain embodiments comprising a different unique identifying sequence) binds to a different target and contains a different ssDNA linker extension. This allows for the highest degree of multiplexing and specificity; however, it may not always be required. In certain embodiments, the same primary nanostructure can be used to detect different targets, allowing for the same ssDNA linker extension to be used to amplify the fluorescence signal for multiple targets. FIGS. 5A-5B shows such an example of multiplexing where primary nanostructure 1 is used to stain targets 1 and 3. Both of these targets can then be amplified with the addition of the same secondary nanostructure 1. This degenerate form of amplification allows researchers to amplify categories of targets in a manner similar to the way secondary antibodies target categories of species and isotype combinations, illustrating the flexibility of the platform if absolute specificity is unnecessary.

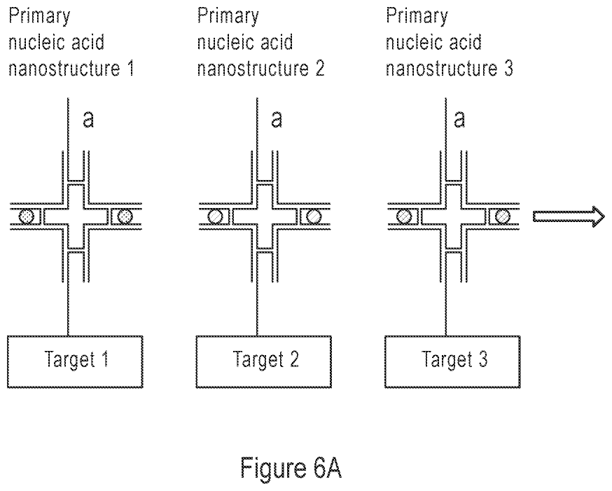

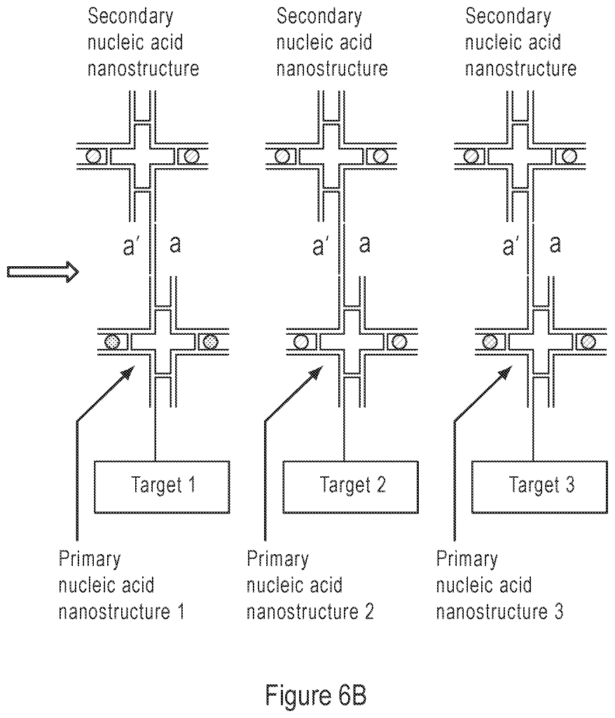

Also provided for herein is the use of degenerate ssDNA linker extensions across different primary nanostructures to convert the spectral profile of those primary nanostructures to an alternative spectral profile. For example, in FIGS. 6A-6B, primary nanostructures 1, 2, and 3 all share the same ssDNA linker extension (a). When a secondary nanostructure is added that contains ssDNA linker extension (a′), every primary nanostructure can now be observed using the same secondary nanostructure spectral profile. While in this example, every primary nanostructure is converted to the same alternative secondary spectral profile, in other embodiments, through mixing and matching different ssDNA linker extensions, different primary nanostructure spectral profiles can be converted to different secondary nanostructure spectral profiles.

In addition to enhanced multiplexing, specificity, and flexibility, the nanostructure compositions and methods of this disclosure enable the amplification factor for primary-secondary nanostructure complexes to be uniquely and controllably tuned. One method for tuning this amplification is through the use of multiple ssDNA linker extensions per nanostructure. For example, in certain embodiments, a primary nanostructure can contain n number of separate ssDNA linker extensions (wherein n can be 2, 3, 4, or more), each of which can bind to a separate corresponding secondary nanostructure. For example, when n is 3, it is contemplated that this can provide 4-fold amplification over the original fluorescence signal of the primary nanostructure since every target molecule is now labeled with four nanostructures (one primary nanostructure and three secondary nanostructures) rather than just the one primary nanostructure.

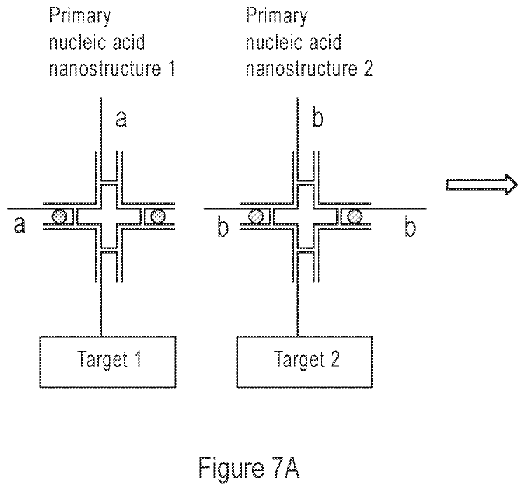

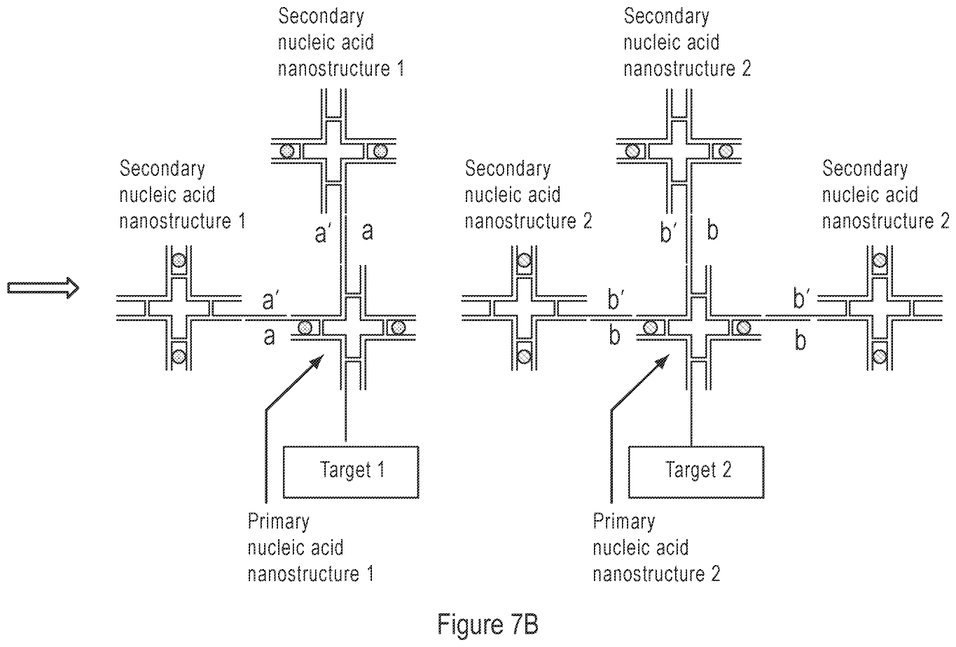

FIGS. 7A-7B show an example of two-way multiplexed amplification in which each target is amplified by a different factor. Targets 1 and 2 are first stained with primary nanostructure 1 and 2 containing ssDNA linker extensions (a) and (b), respectively. Primary nanostructure 1 contains two ssDNA linker extensions and primary nanostructure 2 contains three ssDNA linker extensions. Secondary nanostructures 1 and 2 are added, each carrying one ssDNA extension, (a′) and (b′), respectively. This allows for two copies of secondary nanostructure 1 to bind to primary nanostructure 1 and three copies of secondary nanostructure 2 to bind to primary nanostructure 2. Thus, the amplification factors are controllably set to 3-fold and 4-fold respectively, as targets 1 and 2 are labeled with three and four nanostructures each.

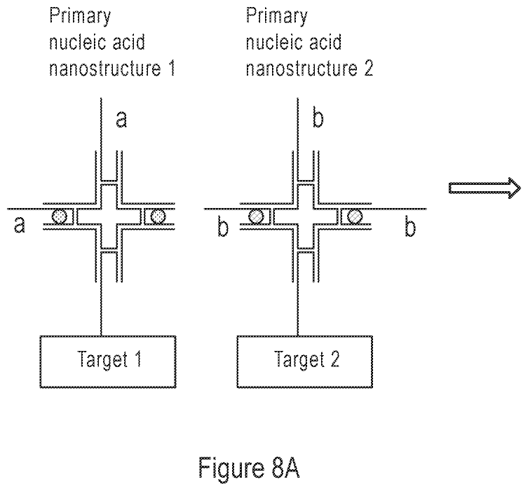

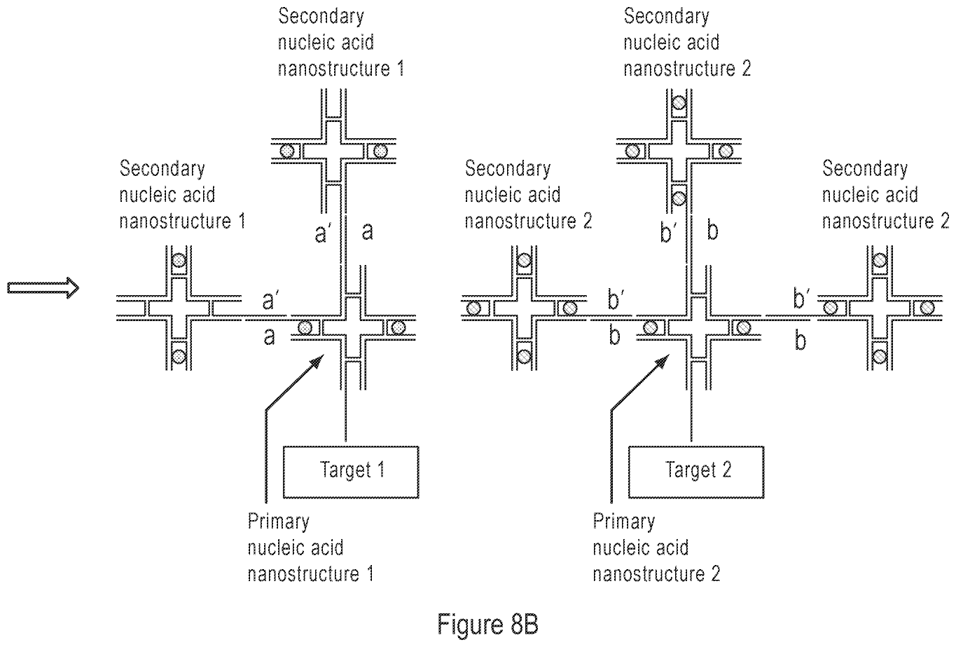

In addition to tuning the amplification factor by controlling the number of secondary nanostructures bound to each primary nanostructure, each primary and secondary nanostructure can be designed to carry with it a different number of fluorophores and/or a combination of fluorophores. FIGS. 8A-8B illustrates a similar example to FIGS. 7A-7B, this time however, each copy of secondary nanostructure 1 contains two fluorophores and each copy of secondary nanostructure 2 contains four fluorophores. This results in a 3-fold amplification of the fluorescence signal for target 1 and a 7-fold amplification of the fluorescence signal for detecting target 2.

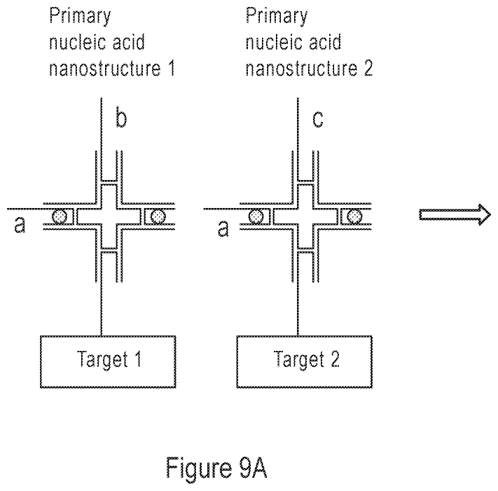

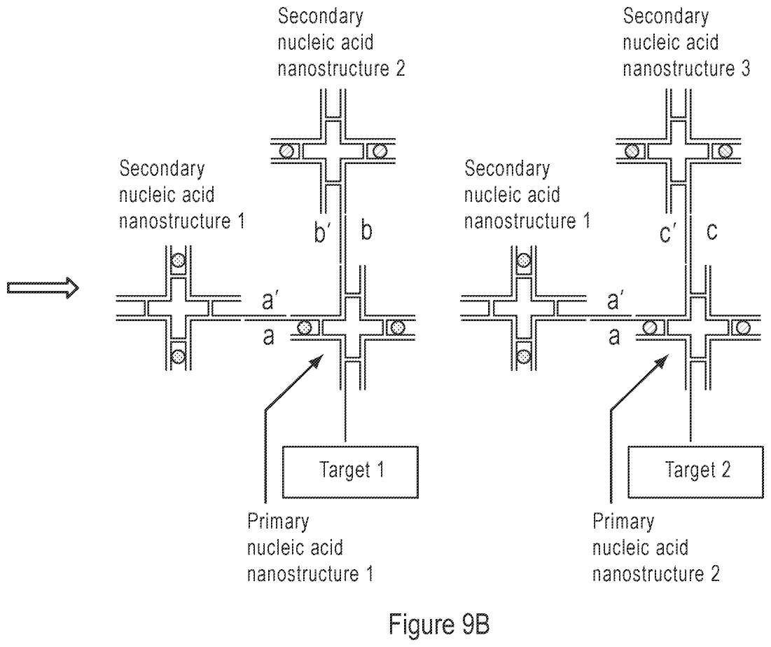

While in certain embodiments utilizing controllable amplification factors, each primary nanostructure has multiple copies of the same ssDNA linker extension, in certain embodiments, the linker extensions are unique, and different secondary nanostructures can bind to each of the primary nanostructure's ssDNA extensions, to form a unique set of secondary nanostructures (and thus a unique combination of fluorophores, a unique spectral profile, and/or unique combination of identifying sequences) attached to a target. This allows for each target molecule to be uniquely labeled (“barcoded”), as well as amplified. FIGS. 9A-9B illustrate an example in which targets 1 and 2 are stained with two primary nanostructures that have the same spectral profile but contain different ssDNA linker extensions. Primary nanostructure 1 contains extensions (a) and (b), while primary nanostructure 2 contains extensions (a) and (c). During amplification, secondary nanostructures 1, 2, and 3, with ssDNA linkers (a′), (b′), and (c′), respectively, bind to the primary nanostructure to create a uniquely coded spectral barcode for each target.

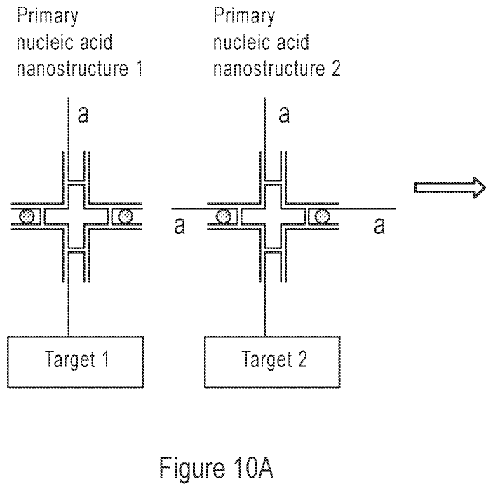

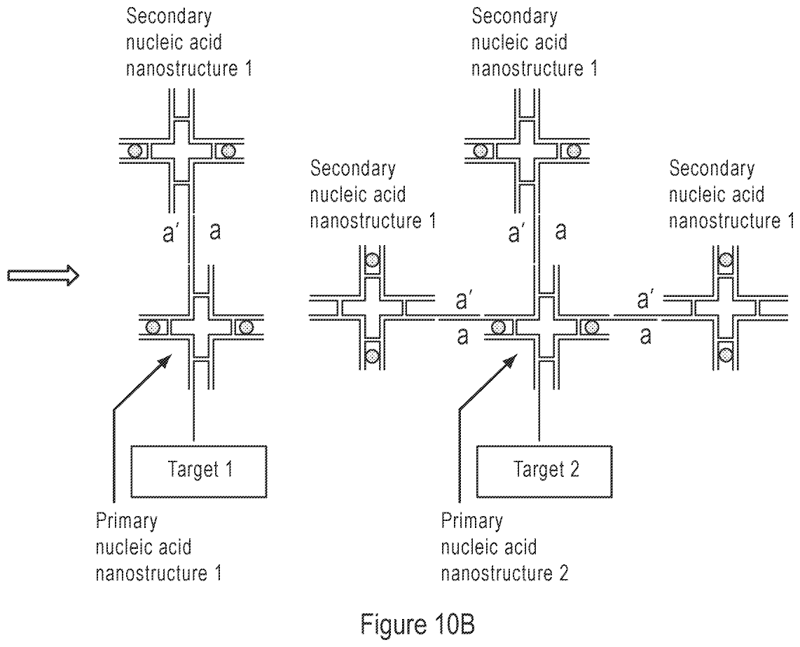

Targets may also be barcoded based on the amplification factor or final resulting signal intensities, which allows for the use of a single spectral profile and/or unique identifying sequence to be used across multiple targets. In FIGS. 10A-10B, for instance, targets 1 and 2 are labeled with the two different primary nanostructures each comprising the same fluorophores but having a different number of ssDNA linker extensions, all of sequence (a). When secondary nanostructures are added and hybridized to the primary nanostructures, target 1 is amplified 2-fold, while target 2 is amplified 4-fold. If the concentration (or density) of the targets is known ahead of time, the different signal intensities of these targets may be used to distinguish them from one another in single particle detection applications even though they share the same spectral profile (and/or unique identifying sequences). In certain embodiments, this method of amplification can simplify the instrumentation needed for detection since only a single laser source and filter set is required to identify multiple targets. In addition, using this embodiment, samples, e.g., from multiple patients, may be uniquely identified and run together as one “sample.”

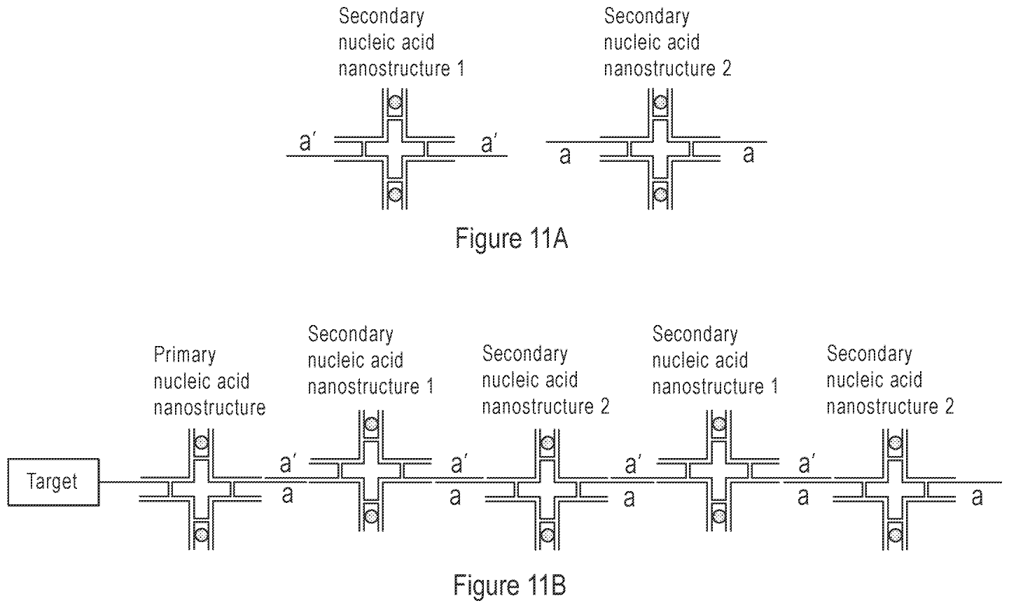

In certain embodiments, even higher degrees of signal amplification can be achieved by incorporating ssDNA linker extensions into the secondary nanostructures in order to form longer, extended networks of nucleic acid nanostructures. FIGS. 11A and 11B provide an example of this form of nanostructure polymerization in which two secondary nanostructures are designed, each having two ssDNA linker extensions of either sequence (a) or sequence (a′). In certain embodiments, a target is first stained with a primary nanostructure containing ssDNA linker extension (a). By adding a mixture of secondary nanostructure 1 and secondary nanostructure 2, a long network of alternating secondary nanostructures can hybridize to provide a significant increase in signal intensity.

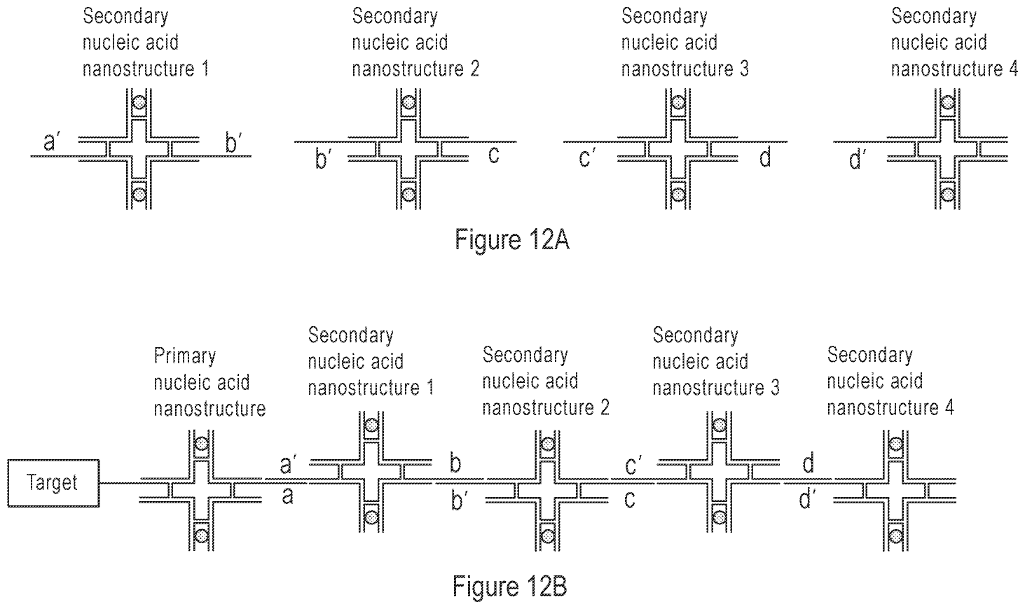

In the above embodiment, it can be difficult to control the degree of polymerization since secondary nanostructures 1 and 2 will continue to polymerize in an alternating fashion without the addition of a terminating species. To better control the degree of polymerization, the ssDNA linker extensions of the secondary nanostructures can be specifically designed to limit the polymer to a certain chain length. FIGS. 12A and 12B show an example of this in which four distinct secondary nanostructures are designed, each with a unique pair of ssDNA linker extensions. These extensions create a rule set in which secondary nanostructure 1 only binds to the primary nanostructure, secondary nanostructure 2 only binds to secondary nanostructures 1 and 3, secondary nanostructure 3 only binds to secondary nanostructures 2 and 4, and secondary nanostructure 4 acts as a terminating secondary nanostructure because it only contains a single ssDNA linker extension that binds to secondary nanostructure 3 exclusively (see FIG. 12B).

FIGS. 12A and 12B demonstrate an example of nucleic acid nanostructure polymerization in which the final length of the polymer is controlled absolutely. By only using each ssDNA sequence once, each polymer is guaranteed to consist of, at most, one primary nanostructure and four secondary nanostructures. While this degree of control can be beneficial, it is sometimes unnecessary, and it requires a unique secondary nanostructure design for each monomer unit. In certain amplification applications, simply limiting the average length of the polymer is sufficient. In these embodiments, secondary nanostructures can be designed to reuse sequences but still terminate the polymerization process, resulting in a mixture of polymer lengths. In certain embodiments, the average length of these polymers can be tuned based on the starting conditions.

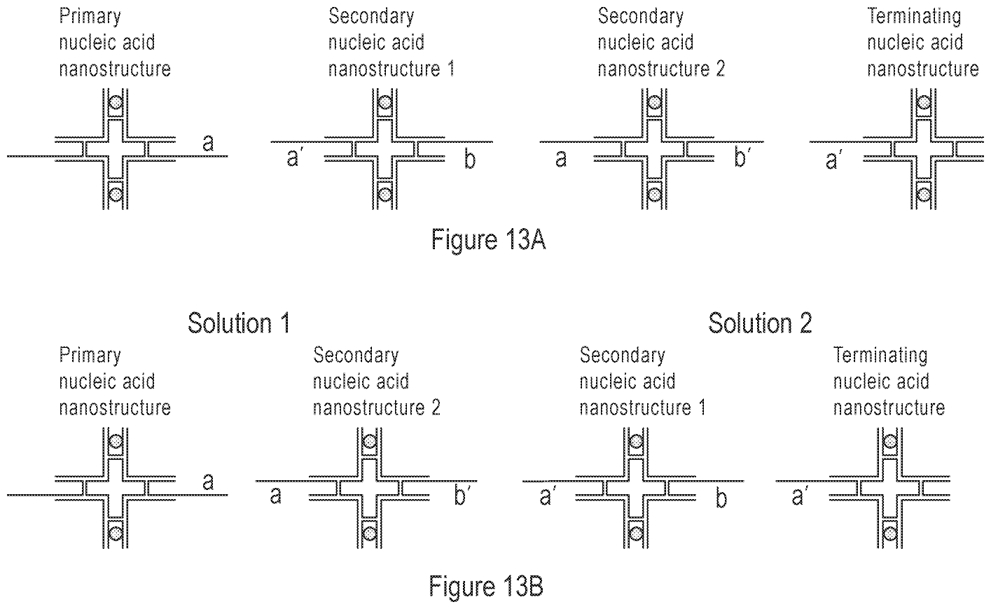

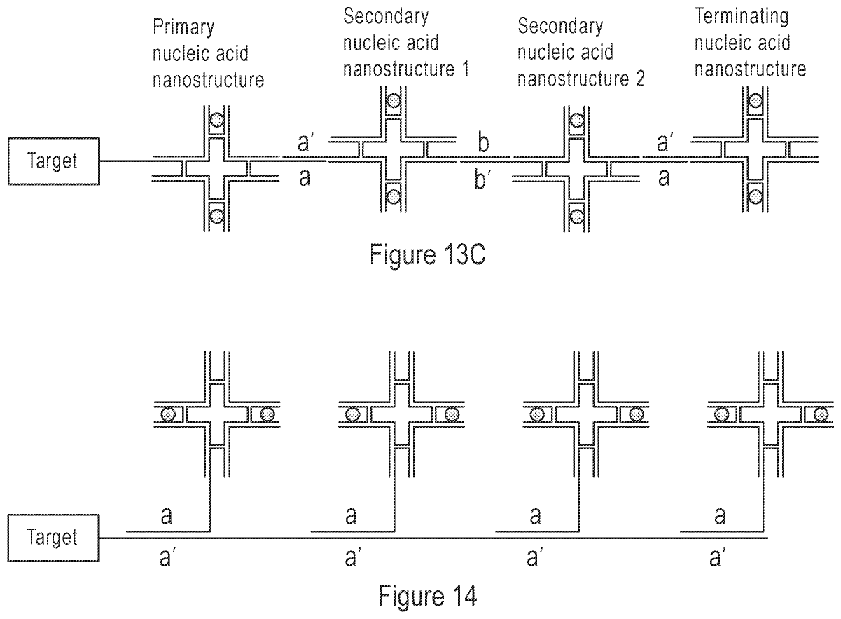

FIGS. 13A-13C show an example embodiment of a semi-controlled nucleic acid nanostructure polymerization method. In this case, there is one primary nanostructure, two secondary nanostructures, and one terminating nanostructure. The primary nanostructure only contains the ssDNA linker extension (a), and the terminating nanostructure only contains the ssDNA linker extension (a′). Secondary nanostructures 1 and 2 contain ssDNA linker extensions (a′) and (b), and (a) and (b′) respectively. The primary nanostructure is first mixed with secondary nanostructure 2 at a specific molar ratio in Solution 1. These two nanostructures will not hybridize as they do not contain any complementary ssDNA linker extensions. For the same reason, the terminating secondary nanostructure is mixed with secondary nanostructure 1, again at a specific molar ratio in Solution 2. When these two solutions are then mixed, polymers of various lengths will form. Some polymers will contain all four nanostructures, as shown in FIG. 13C. Others will consist of only a single primary nanostructure and a single terminating secondary nanostructure. Furthermore, some polymers will consist of a primary nanostructure, followed by a chain of alternating secondary nanostructures 1 and 2, and a terminating secondary nanostructure. This example is not meant to be limiting, and the average length of the resulting polymer distribution can be tuned in a variety of ways, such as changing the stoichiometry regarding the different species, the order in which they are added, and even the times at which the different species are added (e.g., the terminating secondary nanostructure could be added after waiting a specified amount of time).

In certain embodiments, polymer-like structures can be formed using one long coordinating DNA extension (also referred to herein as a “scaffold”) to which are attached multiple nanostructures in order to amplify a signal. FIG. 14 shows an example of this in which one long ssDNA scaffold extension captures four copies of the same nanostructure. The scaffold extending from the target may consist of both ssDNA and double-stranded DNA (dsDNA) regions, which can be used to continue extending its reach for capturing many nanostructures.

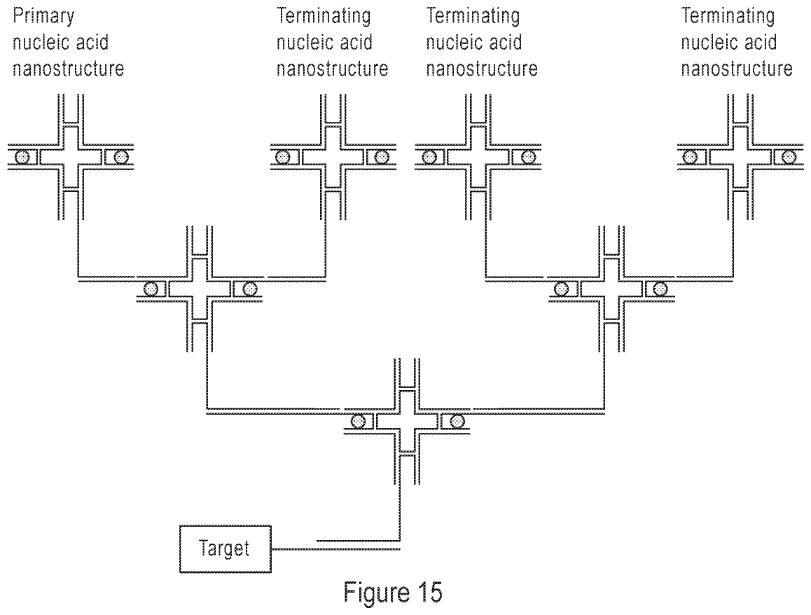

In certain embodiments of this disclosure, a secondary nanostructure can be designed to bind to a primary nanostructure and more than one other secondary nanostructure, more than two other secondary nanostructures, or otherwise nanostructure complexes can form branched networks, providing multiple layers of amplification per target. FIG. 15 illustrates an example in which only a single nanostructure is hybridized to a scaffold attached to the target, but that nanostructure has two ssDNA linker extensions that can then hybridize to other nanostructures. This network of nanostructures can be tuned based on the number of scaffolds and the lengths of these sequences. Ultimately the final network size can be limited by adding terminating nanostructures that only have a single ssDNA linker extension, thereby terminating the growth of the network. One of ordinary skill in the art will understand that while the embodiment illustrated in FIG. 15 shows a branched scaffold attached the target, similarly branched networks of primary-secondary nanostructures complexes as described elsewhere herein are part of this disclosure. In contrast, FIGS. 13C and 14 depict non-limiting examples of linear scaffolds attached to a target.

In the above embodiments, the primary nanostructures to be amplified are described as each containing one or more ssDNA linker extensions before they are bound to the target, i.e., the primary nanostructures are designed ahead of time with the intention of amplifying the signal later. In certain embodiments, however, it is also possible to modify primary nanostructures after binding to the target to reveal ssDNA domains for secondary nanostructure hybridization. This allows researchers to decide whether certain signals should be amplified even after they've already measured the sample, rather than forcing them to decide ahead of time. For example, ssDNA domains on primary nanostructures can be exposed using common, existing dsDNA alteration mechanisms, e.g., CRISPR systems or restriction enzymes such as zinc-finger nucleases. By revealing ssDNA domains, secondary nanostructures that hybridize to these newly exposed regions can be added for on-demand amplification of signals. Conversely, amplification can be stopped or quenched on-demand through the use of DNA polymerases. Rather than expose new sequences, polymerases can extend regions, thereby converting the ssDNA into double helical domains that are no longer accessible to the secondary nanostructures.

One of ordinary skill in the art will recognize that the amplification methods described in this disclosure can be mixed and matched to push the boundaries of signal detection (e.g., fluorescence and/or by sequencing). For instance, through a combination of extended nanostructure networks (FIG. 15), capture strands that polymerize those extended networks (FIG. 14), and increasing the number of fluorophore clusters per nanostructure (FIG. 8), signals can be amplified by multiple orders of magnitude. This degree of amplification can be leveraged to push the limits of detection in applications that require the ability to detect weakly expressed targets, extracellular vesicles, or even single molecules in flow cytometry and a variety of other applications.

The methods of amplification of this disclosure can be used to target a variety of molecular species, including, but not limited to, antigens, antibodies, oligonucleotides, proteins, small particles, aptamers, and extracellular vesicles. These methods can also be used to amplify signals, such as fluorescence and/or sequence signals, in a variety of different applications, including, but not limited to, flow cytometry, microscopy, immunofluorescence, immunochemistry, immunohistochemistry, other forms of imaging, polymerase chain reactions, sequencing, morphology measurements, antibody, and drug screening.

The well-controlled amplification methods outlined in this disclosure and the underlying nanostructure itself (for example, a PHITON nucleic acid nanostructure, which always has a well-defined number of fluorophores attached) can be leveraged to perform quantitative analyses of amplified signals. For instance, primary nanostructures bound to a reference sample with a known number of targets can be amplified by one of the controlled secondary nanostructure amplification methods disclosed herein. Since the number of targets in the reference sample is known, the amount of signal per target can be determined. This can also be correlated with the amount of signal per nanostructure (since the amplification method is controlled and thus the number of nanostructures is known), and finally in certain embodiments the amount of signal per individual fluorophore (since the number of fluorophores per nanostructure is controlled and known). That characterized nanostructure and amplification strategy can then be used to quantify a target that is expressed at an unknown density or suspended at an unknown concentration.

While many exemplary and illustrative embodiments outlined throughout this disclosure may refer to amplifying fluorescence signals, it should be noted that the general concept of controllably increasing the number of nanostructures bound to a specific target also enables directed amplification and localization of “cargo” (e.g. proteins, substrates, drugs, etc.) to that target. For example, primary and secondary nanostructures can include unique identifying nucleic acid sequences to which specific proteins will bind in a transcription factor-like manner to create a molecular complex for detection or effecting protein activity. Thus, the number of proteins bound can be tightly controlled using the quantitative amplification strategies outlined. Furthermore, those bound proteins can be localized to a specific part of the cell based on the target to which the amplified nanostructure network is bound. In these cases, the nanostructure behaves as a vehicle for carrying this cargo in a well-controlled, quantitative manner. It should also be noted that oligonucleotide structures like tRNA can also bind to specific sequences on the nanostructure, enabling an individual or multiple nanostructures to bring biomolecules together for assembly, effector activity, and/or for enhanced detection.

The nucleic acid nanostructure-based amplification compositions and methods disclosed herein raises the upper bound on the degree of multiplexing. It also provides high specificity (when needed), greater flexibility, and the ability to barcode (provide unique identification) the amplification through the introduction of additional spectral profiles and/or tunable fluorescence intensities. Furthermore, the various components and methods of amplification can be mixed and matched with one another to increase the fluorescence and/or sequencing signal by multiple orders of magnitude. In certain embodiments, the nanostructures and/or nanostructure complexes can be incorporated into products such as in the form of a buffer-like solution that can increase the signal of multiple colors simultaneously. In certain embodiments, the compositions and methods of the disclosure can be used in flow cytometry to perform highly multiplexed amplification. In certain embodiments, the compositions and methods of the disclosure can be used to amplify fluorescence signals in e.g., microscopy and single molecule detection and can also be used to create simple but ultra-sensitive diagnostic devices including lateral flow assays.

### Nanostructure Complexes

Provided for herein are nucleic acid nanostructure complexes comprising a primary nucleic acid nanostructure (primary nanostructure) linked to one or more secondary nucleic acid nanostructures (secondary nanostructures). For purposes of this disclosure, a “primary nanostructure” is the nanostructure that is either bound to a target or attached to a specificity determining molecule, such as an antibody, that is bound to a target. A “secondary nanostructure” is a nanostructure that is linked to a primary nanostructure and/or to another nanostructure in the complex. A secondary nanostructure that is adjacently linked to a primary nanostructure is a “proximal secondary nanostructure.” A secondary nanostructure that is adjacently linked to just one other secondary nanostructure is a “terminal secondary nanostructure.” In some embodiments, a terminal secondary nanostructure comprises only one nucleic acid linker extension or otherwise cannot be linked to additional secondary nanostructures. By “adjacently linked,” it is meant that there is a direct attachment or attachment through a linker between the primary nanostructure and the proximal secondary nanostructure but no intervening secondary nanostructure(s). In certain embodiments, a primary nanostructure is adjacently linked to any of between about 1, 2, 3, 4, 5, 6, 7, 8, 9, 10, 15, 20, 25, 30, or 40 to any of about 2, 3, 4, 5, 6, 7, 8, 9, 10, 15, 20, 25, 30, 40, or 50 secondary nanostructures. In certain embodiments, a primary nanostructure is adjacently linked to one, two, three, four, five, six, seven, eight, nine, or ten secondary nanostructures. In certain embodiments, a primary nanostructure is adjacently linked to one, two, three, or four secondary nanostructures. In certain embodiments, a primary nanostructure is linked to one or more secondary nanostructures through one or more intervening secondary nanostructures. In certain embodiments a secondary nanostructure is linked to a primary nanostructure by being linked to an intervening proximal secondary nanostructure that is adjacently linked to the primary nanostructure. In certain embodiments a secondary nanostructure is linked to a primary nanostructure by being linked to one or more intervening secondary nanostructures leading back to a proximal secondary nanostructure adjacently linked to the primary nanostructure.

In certain embodiments, a primary nanostructure itself can specifically bind to a target molecule such as to genomic material. In certain other embodiments, a primary nanostructure is linked to a specificity determining molecule.

In certain embodiments, the specificity determining molecule comprises a protein, enzyme, carbohydrate, nucleic acid, receptor, receptor ligand, and/or substrate that enable the assaying of different -omes (e.g, transcriptome, epigenome, genome, and proteome). In certain embodiments, the specificity determining molecule is a binding molecule such as an antibody or an antigen-binding fragment, variant, or derivative thereof. In certain embodiments, the binding molecule is a peptide, recombinant, natural, or engineered receptor/ligand protein, aptamers, tetramers (folded MHC proteins with peptides used for detecting T cell receptors), non-antibody proteins or antibody mimetics, e.g., affilins, affimers, affitins, alphabodies, avimers, fynomers, Kunitz domain peptides, nanoCLAMPS, Designed Ankyrin Repeat Proteins (DARPins), monobodies, nanobodies, anticalins, affibodies, and/or SOMAmers. In certain embodiments, the binding molecule is a receptor ligand. A specificity determining molecule may be either naturally occurring or synthetic. In certain embodiments, a specificity determining molecule enables the targeting of genomic material. For example, a specificity determining molecule may itself target genomic material, e.g., DNA or RNA, to perform FISH or other biological assays, e.g., on chromatin accessibility or gene expression. In certain embodiments, the reagents and methods of this disclosure provide the ability to use the tools of gene editing to target, expose, and create new amplified structures for detection. For example, various enzymes and modalities of gene editing and targeting, including degenerate systems, e.g., DNAses, and specific targeting, e.g. CRISPR and Zinc-finger nucleases.

In certain embodiments, the primary nanostructure is linked to the specificity determining molecule via a nucleic acid linker. In certain embodiments, the primary nanostructure is linked to a secondary nanostructure via a nucleic acid linker. A nucleic acid linker can be single-stranded, partially single-stranded/double-stranded, or double-stranded. In certain embodiments, the nucleic acid linker is a hybridized at least partially double-stranded linker. As referred to herein, the portion of an at least partially single-stranded linker to be hybridized with a single-stranded portion of a complementary linker is the hybridizing region and when hybridized, the double-stranded sequence is the hybridized region. A nucleic acid linker can comprise or consist of DNA or RNA or an analog thereof such as comprising a synthetic backbone or base. The nucleic acid linker can be of any length, but certain considerations can be taken into account. For example, an extremely short linker may bring conjugate components into too close of contact, resulting in steric hindrance or other interference. On the other hand, a very long linker may be more difficult to produce or may not keep the components within an optimal distance. In certain embodiments, the nucleic acid linker is at least about 10, 11, 12, 13, 14, 15, 16, 17, 18, 19, 20, 25, 30, 35, 40, 50, 60, 65, 70, or 75 nucleotides in length. In certain embodiments, the nucleic acid linker is from any of about 10, 11, 12, 13, 14, 15, 16, 17, 18, 19, 20, 25, 30, 35, 40, 50, or 60 nucleotides in length to any of about 11, 12, 13, 14, 15, 16, 17, 18, 19, 20, 25, 30, 35, 40, 50, 60, or 75 nucleotides in length; In certain embodiments, the nucleic acid linker is from any of about 10, 15, 20, 25, 30, 35, 40, or 50 nucleotides in length to any of about 15, 20, 25, 30, 35, 40, 50, or 75 nucleotides in length. In certain embodiments, the nucleic acid linker is from any of about 15, 20, 25, 30, or 35 nucleotides in length to any of about 20, 25, 30, 35, or 40 nucleotides in length. The nucleic acid linker can include both single-stranded and double-stranded segments. In certain embodiments, the double-stranded segment of the nucleic acid linker is at least about 10, 15, 20, 25, 30, 35, 40, 50, 60, 65, 70, or 75 nucleotides in length. In certain embodiments, the double-stranded segment of the nucleic acid linker is any of about 10, 15, 20, 25, 30, 35, 40, 50, or 60 nucleotides in length to any of about 15, 20, 25, 30, 35, 40, 50, 60, or 75 nucleotides in length. One of ordinary skill in the art will recognize that whereas double-stranded nucleic acids are generally thought to be made of annealed sequences of complementary base pairs, not all the pairing in a double-stranded nucleic acid segment need be complementary. There is some tolerance for two strands of nucleic acids comprising complementary bases to anneal to form a double-stranded nucleic acid incorporating some non-complementary base paring. Also, degenerate (universal) bases such as deoxyinosine exist that can pair with numerous bases. In certain embodiments, the double-stranded segment of the nucleic acid linker comprises at least about 10, 15, 20, 25, 30, 35, 40, 45, 50, 55, 60, 65, 70, or 75 complementary base pairs, even if the double-stranded segment is not entirely composed of complementary base pairs. In certain embodiments, the double-stranded segment of the nucleic acid linker comprises from any of about 10, 15, 20, 25, 30, 35, 40, 50, or 60 complementary base pairs to any of about 15, 20, 25, 30, 35, 40, 50, 60, or 75 complementary base pairs, even if the double-stranded segment is not entirely composed of complementary base pairs. In certain embodiments, at least 85%, 90%, 95%, or 98% of the double-stranded segment of the nucleic acid linker is complementary base paired. In certain embodiments, the double-stranded segment of the nucleic acid linker has no more than 1, 2, 3, 4, 5, 6, 7, 8, 9, 10, or 11 mismatched base pairs. In certain embodiments, however, 100% of the double-stranded segment is complementary base paired. In certain embodiments, the double-stranded segment comprises at least about 7, 8, 9, 10, 11, 12, 13, 14, 15, 16, 17, 18, 19, 20, 25, 30, 40, 50, 60, or 75 consecutive complementary base pairs or from any of about 7, 8, 9, 10, 15, 20, 25, 30, 35, 40, 50, or 60 consecutive complementary base pairs to any of about 8, 9, 10, 15, 20, 25, 30, 35, 40, 50, 60, or 75 consecutive complementary base pairs.

Because a hybridization between two complementary nucleic acid strands is non-covalent, components of a nanostructure complex can be linked together non-covalently. In certain embodiments, the primary nanostructure and the specificity determining molecule are not linked by a covalent bond. In certain embodiments, the primary nanostructure and at least one linked secondary nanostructure are not linked by a covalent bond. And, in certain embodiments, the primary nanostructure and none of the linked secondary nanostructures are linked by a covalent bond.

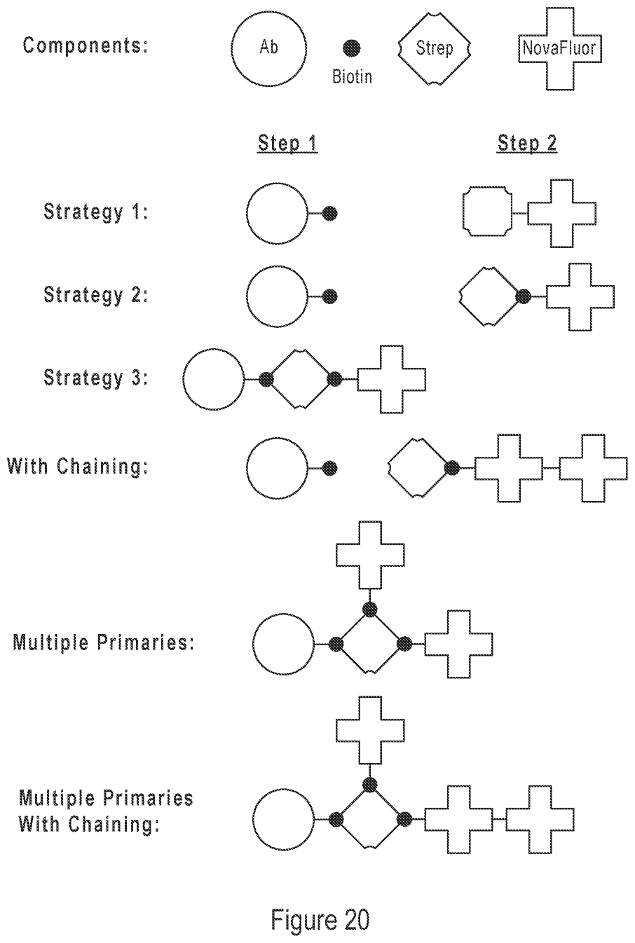

In certain embodiments, the primary nanostructure is linked to the specificity determining molecule via a biotin/biotin-binding protein complex (for example as illustrated in FIG. 20). In certain embodiments, the primary nanostructure is linked to the specificity determining molecule via an avidin-biotin, streptavidin-biotin, or NeutrAvidin-biotin complex. In certain embodiments, the primary nanostructure is linked to the specificity determining molecule via streptavidin-biotin complex. Avidin is a protein derived from both avians and amphibians that shows considerable affinity for biotin, a co-factor that plays a role in multiple eukaryotic biological processes. Avidin and other biotin-binding proteins, including streptavidin and NeutrAvidin, have the ability to bind up to four biotin molecules (FIG. 20). Because the biotin label is stable and small, it rarely interferes with the function of labeled molecules. Because biotin-binding proteins have the ability to bind up to four biotin molecules, in certain embodiments, more than one primary nanostructure is linked to the specificity determining molecule via the same biotin/biotin-binding protein complex (FIG. 20; “Multiple Primaries”). This can be done with respect to any of the approaches described below and is not limited to the particular biotin-binding protein/nanostructure arrangement shown in FIG. 20. Further, in certain embodiments, additional nanostructures can be added to primary nanostructure through chaining (as described elsewhere herein) to provide, for example, amplification of the nanostructure signal (FIG. 20; “Multiple Primaries With Chaining”).

In certain embodiments, the biotin-binding protein (e.g., streptavidin) can be complexed to the nucleic acid nanostructure using amine-reactive labeling chemistry. This approach leaves open all biotin binding sites. This biotin-binding protein/nanostructure complex can be used to label a biotinylated specificity determining molecule, such as a biotinylated antibody, with the nanostructure. For example, via the formation of a biotin/biotin-binding protein complex.

In certain embodiments, the nanostructure can be biotinylated by incorporating a linker oligo modified (such as at the 3′ terminus) with biotin. This combination can then be complexed with the biotin-binding protein (e.g., streptavidin). The complexing can be done in various ratios. This biotin-binding protein/nanostructure complex can be used to label a biotinylated specificity determining molecule, such as a biotinylated antibody, with the nanostructure, for example, via the formation of a biotin/biotin-binding protein complex.

In certain embodiments, the nanostructure can be biotinylated by incorporating a linker oligo modified (such as at the 3′ terminus) with biotin. This combination can then be complexed with the biotin-binding protein (e.g., streptavidin). The complexing can be done in various ratios. This biotin-binding protein/nanostructure complex can then be incubated with a biotinylated specificity determining molecule (e.g., an antibody) to form a specificity determining molecule/biotin-binding protein/biotinylated-nanostructure complex (e.g., an antibody/streptavidin/biotinylated-nucleic acid nanostructure complex). This pre-assembled complex can be used as a primary stain in flow cytometry.

Illustrative examples of the structure of various nanostructure complexes of this disclosure are shown in the Figures and described in the Examples. In certain embodiments a nanostructure complex comprises the general structure:

P(-L-S)ₙ

wherein P is the primary nanostructure, L is a hybridized at least partially double-stranded nucleic acid linker, S is one or more adjacently linked secondary nanostructures, and n is an integer greater than zero, wherein (i) the primary nanostructure comprises n number of partially single-stranded nucleic acid linker extensions, (ii) there are n number of secondary nanostructures comprising an at least partially single-stranded nucleic acid linker extension, and (iii) at least a portion of the nucleic acid sequence of the single-stranded region of the n number of primary nanostructure nucleic acid linker extensions is at least partially complementary to at least a portion of the nucleic acid sequence of the single-stranded region of one of the secondary nucleic acid linker extensions sufficient to form a hybridized at least partially double-stranded linker between the primary nanostructure and each of the secondary nanostructures, thus linking the primary nanostructure to n number of secondary nanostructures. In certain embodiments, n is between about 1, 2, 3, 4, 5, 6, 7, 8, 9, 10, 15, 20, 25, 30, or 40 to any of about 2, 3, 4, 5, 6, 7, 8, 9, 10, 15, 20, 25, 30, 40, or 50. In certain embodiments, n is one, two, three, four, five, six, seven, eight, nine, or ten. In certain embodiments, n is one, two, three, or four.

In certain embodiments the primary nanostructure is adjacently linked to one secondary nanostructure. For example, in certain embodiments the primary nanostructure is adjacently linked to one secondary nanostructure and (i) the primary nanostructure comprises an at least partially single-stranded nucleic acid linker extension, (ii) the secondary nanostructure comprises an at least partially single-stranded nucleic acid linker extension, and (iii) at least a portion of the nucleic acid sequence of the single-stranded region of the primary nanostructure nucleic acid linker extension is at least partially complementary to at least a portion of the nucleic acid sequence of the single-stranded region of the secondary nanostructure nucleic acid linker extension sufficient to form a hybridized at least partially double-stranded linker, thus linking the primary nanostructure to the secondary nanostructure.

In certain embodiments the primary nanostructure is adjacently linked to two secondary nanostructures via linkers comprising the same hybridizing/hybridized regions. For example, in certain embodiments the primary nanostructure is adjacently linked to two secondary nanostructures and (i) the primary nanostructure comprises two at least partially single-stranded nucleic acid linker extensions comprising the same single-stranded hybridizing region sequence, (ii) both secondary nanostructures comprise an at least partially single-stranded nucleic acid linker extension, optionally, comprising the same single-stranded hybridizing region sequence, and (iii) at least a portion of the nucleic acid sequence of the single-stranded hybridizing region of the primary nanostructure nucleic acid linker extensions is at least partially complementary to at least a portion of the nucleic acid sequence of the single-stranded hybridizing region of the secondary nanostructures' nucleic acid linker extensions, sufficient to form a hybridized at least partially double-stranded linker between the primary nanostructure and both secondary nanostructures, thus linking the primary nanostructure to the two secondary nanostructures. In certain embodiments, both secondary nanostructures comprise the same single-stranded hybridizing region sequence.

In certain embodiments the primary nanostructure is adjacently linked to three secondary nanostructures via linkers comprising the same hybridizing/hybridized regions. For example, in certain embodiments the primary nanostructure is adjacently linked to three secondary nanostructures and (i) the primary nanostructure comprises three at least partially single-stranded nucleic acid linker extensions comprising the same single-stranded hybridizing region sequence, (ii) all three secondary nanostructures comprise an at least partially single-stranded nucleic acid linker extension, and (iii) at least a portion of the nucleic acid sequence of the single-stranded hybridizing region of the primary nanostructure nucleic acid linker extensions is at least partially complementary to at least a portion of the nucleic acid sequence of the single-stranded hybridizing region of the secondary nanostructures nucleic acid linker extensions, sufficient to form a hybridized at least partially double-stranded linker between the primary nanostructure and all three secondary nanostructures, thus linking the primary nanostructure to the three secondary nanostructures. In certain embodiments, at least two and/or all three secondary nanostructures comprises the same single-stranded hybridizing region sequence.

In certain embodiments the primary nanostructure is adjacently linked to two secondary nanostructures via linkers comprising different hybridizing/hybridized regions. For example, in certain embodiments the primary nanostructure is adjacently linked to two secondary nanostructures and (i) the primary nanostructure comprises two at least partially single-stranded nucleic acid linker extensions, wherein each of the at least partially single-stranded nucleic acid linker extensions comprises a different single-stranded hybridizing region sequence, (ii) both secondary nanostructures comprise an at least partially single-stranded nucleic acid linker extension, wherein each of the at least partially single-stranded nucleic acid linker extensions comprises a different single-stranded hybridizing region sequence, and (iii) at least a portion of the nucleic acid sequence of the single-stranded hybridizing region of each of the primary nanostructure nucleic acid linker extensions is at least partially complementary to at least a portion of the nucleic acid sequence of the single-stranded hybridizing region of one of the secondary nanostructures nucleic acid linker extensions, sufficient to form a hybridized at least partially double-stranded linker between the primary nanostructure and both secondary nanostructures, thus linking the primary nanostructure to the two secondary nanostructures.

In certain embodiments the primary nanostructure is adjacently linked to three secondary nanostructures via linkers comprising different hybridizing/hybridized regions. For example, in certain embodiments the primary nanostructure is adjacently linked to three secondary nanostructures and wherein (i) the primary nanostructure comprises three at least partially single-stranded nucleic acid linker extensions, wherein at least one of the at least partially single-stranded nucleic acid linker extensions comprises a different single-stranded hybridizing region sequence, (ii) all three secondary nanostructures comprise an at least partially single-stranded nucleic acid linker extension, wherein at least one of the partially single-stranded nucleic acid linker extensions comprises a different single-stranded hybridizing region sequence, and (iii) at least a portion of the nucleic acid sequence of the single-stranded hybridizing region of the each of the primary nanostructure nucleic acid linker extensions is at least partially complementary to at least a portion of the nucleic acid sequence of the single-stranded hybridizing region of one of the secondary nanostructures nucleic acid linker extensions, sufficient to form a hybridized at least partially double-stranded linker between the primary nanostructure and all three secondary nanostructures, thus linking the primary nanostructure to the three secondary nanostructures. In certain embodiments, the primary nanostructure comprises three at least partially single-stranded nucleic acid linker extensions, and at least two and/or each of the at least partially single-stranded nucleic acid linker extensions comprises a different single-stranded hybridizing region sequence. In certain embodiments, at least two and/or each of the partially single-stranded nucleic acid linker extensions of the secondary nanostructures comprises a different single-stranded hybridizing region sequence.

One of ordinary skill in the art would understand from the preceding representative embodiments that primary nanostructures can be linked to additional adjacent secondary nanostructures (also referred to as proximal secondary nanostructures) through nucleic acid linkers comprising the same or different hybridizing/hybridized regions.

In certain embodiments, the primary nanostructure comprises one or a combination of fluorophore moieties that give the nanostructure a spectral profile. In certain embodiments, the secondary nanostructure comprises one or a combination of fluorophore moieties that give the nanostructure a spectral profile. For example, a primary and/or secondary nanostructure can comprise from any of about 1, 2, 3, 4, 5, 6, 7, 8, 9, 10, 11, 12, 13, 14, 15, 16, 17, 18, 19, 20, 21, 22 or 23 fluorophore moieties to any of about 2, 3, 4, 5, 6, 7, 8, 9, 10, 11, 12, 13, 14, 15, 16, 17, 18, 19, 20, 21, 22, 23 or 24 fluorophore moieties. In certain embodiments, the primary nanostructure and at least one of the linked secondary nanostructures both comprise one or a combination of fluorophore moieties that give the nanostructures a spectral profile. In certain embodiments, the primary nanostructure and each of the linked secondary nanostructures all comprise one or a combination of fluorophore moieties that give the nanostructures a spectral profile. In certain embodiments, the primary nanostructure and at least one of the linked secondary nanostructures in combination give the nanostructure complex a spectral profile. In certain embodiments, the primary nanostructure and all of the linked secondary nanostructures, in some embodiments whether adjacently linked to the primary nanostructure or through one or more intervening secondary nanostructures, in combination give the nanostructure complex its spectral profile.

In certain embodiments, the primary nanostructure comprises a unique identifying sequence. In certain embodiments, the secondary nanostructure comprises a unique identifying sequence. In certain embodiments, the primary nanostructure and at least one of the linked secondary nanostructures both comprise a unique identifying sequence. In certain embodiments, the primary nanostructure and each of the linked secondary nanostructures all comprise a unique identifying sequence. In certain embodiments, the primary nanostructure and/or at least one of the linked secondary nanostructures comprises two or more unique identifying sequences. In certain embodiments, a primary and/or secondary nanostructure can comprise any of about 1, 2, 3, 4, 5, 6, 7, 8, 9, 10, 11, 12, 13, 14, 15, 16, 17, 18, 19, 20, 21, 22 or 23 unique identifying sequences to any of about 2, 3, 4, 5, 6, 7, 8, 9, 10, 11, 12, 13, 14, 15, 16, 17, 18, 19, 20, 21, 22, 23 or 24 unique identifying sequences. In certain embodiments, the unique identifying sequence may be incorporated into the nucleic acid linker extension of a nanostructure and for purposes of a nanostructure comprising a unique identifying sequence are considered a part of the nanostructure.

In certain embodiments, a primary nanostructure comprises a unique identifying sequence and one or a combination of fluorophore moieties that give the primary nanostructure a spectral profile. In certain embodiments, at least one of the linked secondary nanostructures comprises a unique identifying sequence and one or a combination of fluorophore moieties that give the secondary nanostructure a spectral profile. In certain embodiments, each of the linked secondary nanostructures comprises a unique identifying sequence and one or a combination of fluorophore moieties that give the secondary nanostructures a spectral profile.

In certain embodiments, a primary nanostructure comprises one or a combination of fluorophore moieties that give the primary nanostructure a spectral profile and is linked to n number of secondary nanostructures having the same spectral profile. The secondary nanostructures with the same spectral profile can amplify the fluorescence signal of the nanostructure complex in comparison to the fluorescence signal of the primary nanostructure alone. Signal amplification as disclosed herein can bring the signal of a labeled molecule or structure within the detection limits of currently available instrumentation even if not previously possible. Such amplification has a broader application than just cells and it is contemplated to be used to examine the currently undetectable, e.g., nanocrystals, supramolecular complexes, extracellular vesicles. Additionally, in certain embodiments this amplification can enable single molecule detection by fluorescence or genomic measurements.

In certain embodiments, the amount of amplification is tunable and can be precisely controlled by the addition of additional nanostructures to the nanostructure complex. In certain embodiments, secondary nanostructures having the same spectral profile as the primary nanostructure also each comprise the same number of fluorophore moieties as the primary nanostructure and amplify the fluorescence signal of the nanostructure complex by a factor of about n+0.5, n+0.6, n+0.7, n+0.8, n+0.9 or n+1 times the amount of the fluorescence signal of the primary nanostructure alone. For example, in certain embodiments the primary nanostructure comprises one or a combination of fluorophore moieties that give the primary nanostructure a spectral profile and is linked to one secondary nanostructure comprising the same spectral profile and the same number of fluorophore moieties as the primary nanostructure, wherein the secondary nanostructure amplifies the fluorescence signal of the nanostructure complex by a factor of about 2 times the amount of the fluorescence signal of the primary nanostructure alone. For example, in certain embodiments the primary nanostructure comprises one or a combination of fluorophore moieties that give the primary nanostructure a spectral profile and is linked to two secondary nanostructures each comprising the same spectral profile and the same number of fluorophore moieties as the primary nanostructure; wherein the two secondary nanostructures amplify the fluorescence signal of the nanostructure complex by a factor of about 3 times the amount of the fluorescence signal of the primary nanostructure alone. And, for example, in certain embodiments the primary nanostructure comprises one or a combination of fluorophore moieties that give the primary nanostructure a spectral profile and is linked to three secondary nanostructures each comprising the same spectral profile and the same number of fluorophore moieties as the primary nanostructure; wherein the three secondary nanostructures amplify the fluorescence signal of the nanostructure complex by a factor of about 4 times the amount of the fluorescence signal of the primary nanostructure alone. One of ordinary skill in the art will understand from the preceding representative embodiments that additional amplification can be achieved in a quantitatively controlled manner by linking additional secondary nanostructures to the primary nanostructure either adjacently or through one or more intervening secondary nanostructures.

Similarly, and as would be understood from above, in certain embodiments the primary nanostructure comprises a unique identifying sequence and is linked to n number of secondary nanostructures comprising the same unique identifying sequence. The secondary nanostructures with the same unique identifying sequence amplify the sequencing signal of the nanostructure complex in comparison to the sequencing signal of primary nanostructure alone. In certain embodiments, the secondary nanostructures also each comprise the same number of unique identifying sequences as the primary nanostructure and amplify the sequencing signal of the nanostructure complex by a factor of about n+0.5, n+0.6, n+0.7, n+0.8, n+0.9 or n+1 times the amount of the sequencing signal of the primary nanostructure alone.

Not all embodiments of this disclosure necessarily result in amplification of a signal. For example, in certain embodiments the primary nanostructure comprises one or a combination of fluorophore moieties and is linked either adjacently or through one or more intervening secondary nanostructures to a secondary nanostructure comprising one or more quenchers that absorb the fluorescence of and/or undergo Forster resonance energy transfer with the fluorophore moieties of the primary nanostructure. In certain embodiments, the primary nanostructure comprises one or a combination of fluorophore moieties that give the primary nanostructure a spectral profile and is linked to a secondary nanostructure comprising a different fluorophore moiety or different combination of fluorophore moieties that give the secondary nanostructure and/or the nanostructure complex a spectral profile that is different from the primary nanostructure spectral profile. That is, the addition of the secondary nanostructure can change the spectral profile such that the spectral profile of the nanostructure complex is different from the spectral profile of the primary nanostructure. In certain embodiments, the secondary nanostructure comprises a fluorophore moiety or combination of fluorophore moieties that can undergo Forster resonance energy transfer with the fluorophore moiety or combination of fluorophore moieties of the primary nanostructure. In certain embodiments, the primary nanostructure is linked to at least two secondary nanostructures each comprising a different fluorophore moiety or different combination of fluorophore moieties that give each secondary nanostructure and/or the nanostructure complex a spectral profile that is different from the primary nanostructure spectral profile. In certain embodiments, at least one of the linked secondary nanostructures comprises a different fluorophore moiety or different combination of fluorophore moieties that give such secondary nanostructure a spectral profile that is different from another linked secondary nanostructure spectral profile. In certain embodiments, each of the linked secondary nanostructures comprises a different fluorophore moiety or different combination of fluorophore moieties that give each secondary nanostructure a spectral profile that is different from any other linked secondary nanostructure spectral profile.

In certain embodiments, the amount of amplification is even more precisely tunable and controllable by controlling the number of fluorophores or combinations of fluorophores of the nanostructure complex. For example, in certain embodiments, the primary nanostructure comprises x number of a fluorophore moiety or a combination of fluorophore moieties and is linked to y number of secondary nanostructures each comprising z number of the same fluorophore moiety or combination of fluorophore moieties as the primary nanostructure, wherein z can be independently determined for each secondary nanostructure and the sum of z from the secondary nanostructures is zₜₒₜₐₗ. In certain embodiments, the fluorescent signal of the nanostructure complex compared to the fluorescent signal of the primary nanostructure alone is amplified by a factor of about (x+zₜₒₜₐₗ)/x. In certain embodiments, z is the same for each secondary nanostructure and zₜₒₜₐₗ=y*z.

Certain embodiments of this disclosure provide for polymers of nanostructures. For examples, a primary nucleic acid nanostructure can be adjacently linked to one or more proximal secondary nucleic acid nanostructures and at least one of the proximal secondary nanostructures is further linked to another secondary nanostructure. As described in greater detail elsewhere herein, in certain embodiments the primary nanostructure and the one or more proximal secondary nanostructures and/or the one or more proximal secondary nanostructures and the another secondary nanostructure linked to the proximal secondary nanostructure can be linked via a hybridized at least partially double-stranded nucleic acid linker. In certain embodiments of nanostructure polymerization, at least one nanostructure of the complex is a fluorescent nanostructure and/or comprises a unique identifying sequence. In certain embodiments, polymerization can be used to amplify a signal, for example wherein the primary nanostructure, the proximal secondary nanostructure, and/or the another secondary nanostructure have at least the same spectral profile, sequencing signal, and/or fluorescence or sequence signal intensity. In certain embodiments the primary nanostructure, the proximal secondary nanostructure, and the another secondary nanostructure all have at least the same spectral profile, sequencing signal, and/or fluorescence or sequence signal intensity. In certain embodiments, polymerization can be used to adjust or modify a signal, for example wherein the primary nanostructure, the proximal secondary nanostructure, and/or the another secondary nanostructure have different spectral profiles, sequencing signal, and/or fluorescence or sequence signal intensities. In certain embodiments the primary nanostructure, the proximal secondary nanostructure, and the another secondary nanostructure all have different spectral profiles, sequencing signals, and/or fluorescence or sequence signal intensities.

In certain embodiments, a nanostructure complex comprises the general formula:

P-L₁-S_{P}-L₂-(Sₓ-L_{y})ₙ-Z

wherein P is the primary nanostructure, L₁ is a linker linking the primary nanostructure to a secondary nanostructure, S_{P} (proximal secondary nanostructure) is a secondary nanostructure adjacently linked to the primary nanostructure, L₂ is a linker linking S_{P} to another secondary nanostructure, n is zero or a positive integer, (Sₓ-L_{y}) comprises a secondary nanostructure Sₓ and linker L_{y} linking Sₓ to an additional secondary nanostructure; and Z is an additional one or more secondary nanostructures. In certain embodiments, Z is a terminal secondary nanostructure S_{T}.

It will be understood that numerous combinations of primary nanostructures, proximal secondary nanostructures, other nanostructures, and/or terminal nanostructures along with the nucleic acid linkers linking them are possible, representative, but not limiting, examples of which are provided for illustration. In certain embodiments the various nanostructures can be the same or at least have the same spectral profile, sequencing signal, and/or fluorescence or sequence signal intensity and can be linked by the same linkers or linkers that at least comprise the same hybridized region sequences. Similarly, the various nanostructures can be different or at least have different spectral profiles, sequencing signals, and/or fluorescence or sequence signal intensities and can be linked by different linkers. For example, in certain embodiments P is the same and/or has the same spectral profile, sequencing signal, and/or fluorescence or sequence signal intensity as Sₚ, S_{T}, and/or at least one or all of Sₓ. In certain embodiments P is different from Sₚ, S_{T}, and/or one or all of Sₓ. For example, in certain embodiments Sₚ is the same or has the same spectral profile, sequencing signal, and/or fluorescence or sequence signal intensity as P, S_{T}, and/or at least one or all of Sₓ. For example, in certain embodiments Sₚ is different from P, S_{T}, and/or one or all of Sₓ. For example, in certain embodiments S_{T} is the same or has the same spectral profile, sequencing signal, and/or fluorescence or sequence signal intensity as P, Sₚ, and/or at least one or all of Sₓ. For example, in certain embodiments S_{T} is different from P, Sₚ, and/or one or all of Sₓ.

In certain embodiments, L₁, L₂, and/or one or more of L_{y} are a hybridized at least partially double-stranded nucleic acid linker. In certain embodiments, L₁ and L₂ comprise the same hybridized region sequence. In certain embodiments, L₁ and L₂ comprise different hybridized region sequences. In certain embodiments, L₁ and L_{y} comprise the same hybridized region sequence. L₁ and L_{y} comprise different hybridized region sequences. In certain embodiments, L₂ and L_{y} comprise the same hybridized region sequence. In certain embodiments, L₂ and L_{y} comprise different hybridized region sequence. In certain embodiments, L₁, L₂, and L_{y} all comprise the same hybridized region sequence. And, in certain embodiments, L₁, L₂, and L_{y} each comprise different hybridized region sequences.

In certain embodiments, n is zero and the nanostructure complex comprises the general formula: P-L₁-S_{P}-L₂-Z. In certain embodiments, Z is S_{T} (e.g., P-L₁-S_{P}-L₂-S_{T}).

In certain embodiments, n is one and the nanostructure complex comprises the general formula: P-L₁-S_{P}-L₂-Sₓ-L_{y}-Z. In certain embodiments, Z is S_{T} (e.g., P-L₁-S_{P}-L₂-Sₓ-L_{y}-S_{T}).

In certain embodiments, n is two and the nanostructure complex comprises the general formula: P-L₁-S_{P}-L₂-S_{x[a]}-L_{y[a]}-S_{x[b]}-L_{y[b]}-Z. In certain embodiments, Z is S_{T} (e.g., P-L₁-S_{P}-L₂-S_{x[a]}-L_{y[a]}-S_{x[b]}-L_{y[b]}-S_{T}). In certain embodiments, (S_{x[a]}-L_{y[a]}) and (S_{x[b]}-L_{y[b]}) are the same. In certain embodiments, S_{x[a]} and S_{x[b]} are the same or at least have the same spectral profile, sequencing signal, and/or fluorescence or sequence signal intensity. In certain embodiments, L_{y[a]} and L_{y[b]} are the same or at least comprise the same hybridized region sequence(s).

In certain embodiments, n is three and the nanostructure complex comprises the general formula: P-L₁-S_{P}-L₂-S_{x[a]}-L_{y[a]}-S_{x[b]}-L_{y[b]}-S_{x[c]}-L_{y[c]}-Z. In certain embodiments, Z is S_{T} (e.g., P-L₁-S_{P}-L₂-S_{x[a]}-L_{y[a]}-S_{x[b]}-L_{y[b]}-S_{x[c]}-L_{y[c]}-S_{T}). In certain embodiments, at least two of (S_{x[a]}-L_{y[a]}), (S_{x[b]}-L_{y[b]}), and (S_{x[c]}-L_{y[c]}) are the same. In certain embodiments, all of (S_{x[a]}-L_{y[a]}), (S_{x[b]}-L_{y[b]}), and (S_{x[c]}-L_{y[c]}) are the same. In certain embodiments, at least two of S_{x[a]}, S_{x[b]}, and S_{x[c]} are the same or at least have the same spectral profile, sequencing signal, and/or fluorescence or sequence signal intensity. In certain embodiments, all of S_{x[a]}, S_{x[b]}, and S_{x[c]} are the same or at least have the same spectral profile, sequencing signal, and/or fluorescence or sequence signal intensity. In certain embodiments, at least two of L_{x[a]}, L_{x[b]}, and L_{x[c]} are the same or at least comprise the same hybridized regions sequence. In certain embodiments, all of L_{x[a]}, L_{x[b]}, and L_{x[c]} are the same or at least comprise the same hybridized region sequence.

Further, in any polymerized nanostructure disclosed herein, in certain embodiments, P is the same or at least has the same spectral profile, sequencing signal, and/or fluorescence or sequence signal intensity as Sₚ, S_{x[a]}, S_{x[b]}, S_{x[c]}, and/or S_{T}. In certain embodiments, P is different from Sₚ, S_{x[a]}, S_{x[b]}, S_{x[c]} and/or S_{T}. In certain embodiments, Sₚ is the same or at least has the same spectral profile, sequencing signal, and/or fluorescence or sequence signal intensity as P, S_{T}, S_{x[a]}, S_{x[b]}, and/or S_{x[c]}. In certain embodiments, Sₚ is different from P, S_{T}, S_{x[a]}, S_{x[b]}, and/or S_{x[c]}. In certain embodiments, S_{T} is the same or at least has the same spectral profile, sequencing signal, and/or fluorescence or sequence signal intensity as P, Sₚ, S_{x[a]}, S_{x[b]}, and/or S_{x[c]}. In certain embodiments, S_{T} is different from P, Sₚ, S_{x[a]}, S_{x[b]}, and/or S_{x[c]}.

Further, in any polymerized nanostructure disclosed herein, in certain embodiments, L₁, L₂, and L_{y} each comprise a different hybridized region sequence and n is two or more and at least two L_{y} comprise the same hybridized region sequence. In certain embodiments, L₁, L₂, and L_{y} each comprise a different hybridized region sequence and n is two or more and all L_{y} comprise the same hybridized region sequence. In certain embodiments, L₁, L₂, and L_{y} each comprise a different hybridized region sequence and n is two or more and all L_{y}, except for the L_{y} linking Sₓ to S_{T}, comprise the same hybridized region sequence. In certain embodiments, L₁ is the same or at least comprises the same hybridized region sequence as L₂, L_{y[a]}, L_{y[b]}, and/or L_{y[c]}. In certain embodiments, L₁ comprises different hybridized region sequences from L₂, L_{y[a]}, L_{y[b]}, and/or L_{y[c]}. In certain embodiments, L₂ is the same or at least comprises the same hybridized region sequence as L₁, L_{y[a]}, L_{y[b]}, and/or L_{y[c]}. In certain embodiments, L₂ comprises a different hybridized region sequence from L₁, L_{y[a]}, L_{y[b]}, and/or L_{y[c]}. In certain embodiments, L₁ comprises a different hybridized region sequence from L₂, and L₁ and L₂ comprises different hybridized region sequences from any of L_{y[a]}, L_{y[b]}, and L_{y[c]}. In certain embodiments, L₁ comprises a different hybridized region sequence from L₂, L₁ and L₂ comprise different hybridized region sequences from any of L_{y[a]}, L_{y[b]}, and L_{y[c]}, and, L_{y[a]}, L_{y[b]}, and L_{y[c]} are the same or at least comprise the same hybridized region sequence. In certain embodiments, L₁ comprises different hybridized region sequence from L₂, L₁ and L₂ comprise different hybridized region sequences from any of L_{y[a]}, L_{y[b]}, and L_{y[c]}, and, L_{y[a]}, L_{y[b]}, and L_{y[c]} are the same or at least comprise the same hybridized region sequence except if L_{y[c]} links Sₓ to S_{T}, L_{y[c]} comprises a different hybridized region sequence from L_{y[a]} and L_{y[b]}.

One of ordinary skill in the art will understand that additional L_{y} linkers can be added in a manner consistent with the illustrative examples above. Further, it would be understood that by controlling the hybridizing region sequences of the at least partially single-stranded nucleic acid linker extensions of the various nanostructures and thus the hybridized regions within the nanostructure complex, the identity and/or order of secondary nanostructures attached to a primary nanostructure is determined by the hybridizing region sequences of the at least partially single-stranded linker extensions of the secondary nanostructures.

In certain embodiments, a terminal secondary nanostructure cannot be linked by hybridization to an additional secondary nanostructure. For example, in certain embodiments, the terminal secondary nanostructure only comprises one at least partially single-stranded linker extension for hybridization to another secondary nanostructure. In certain embodiments, at least one secondary nanostructure comprises one or more quenchers that absorb the fluorescence of and/or undergo Forster resonance energy transfer with the fluorophore moieties of another nanostructure. In certain embodiments, it is a terminal secondary nanostructure that comprises one or more quenchers that absorb the fluorescence of and/or undergo Forster resonance energy transfer with the fluorophore moieties of another nanostructure.

In certain embodiments of the polymerized nanostructures of this disclosure, secondary nanostructures can be polymerized into branched networks. For example, in certain embodiments, at least one proximal secondary nanostructure is adjacently linked to two or more other secondary nanostructures such that the proximal secondary nanostructure is adjacently linked to three other nanostructures. In certain embodiments, at least one non-proximal secondary nanostructure is adjacently linked to three or more other secondary nanostructures.

Further provided herein are complexes of nanostructures assembled on a nucleic acid scaffold (see, for example FIGS. 13C and 15). In certain embodiments, a nanostructure complex comprises a nucleic acid scaffold to which is attached at least one primary nucleic acid nanostructure, wherein the primary nanostructure comprises an at least partially single-stranded nucleic acid linker extension that is hybridized to at least a portion of sequence of the nucleic acid scaffold. In certain embodiments, the nucleic acid scaffold is linked to a specificity determining molecule. In certain embodiments, the complex is bound to a target via the specificity determining molecule. For the purposes of embodiments utilizing a nucleic acid scaffold, any nanostructure adjacently attached to the scaffold is considered a primary nanostructure. In certain embodiments, at least two primary nanostructures are attached to the nucleic acid scaffold via hybridization. In certain embodiments at least two of the at least two primary nanostructures are the same or at least have the same spectral profile, sequencing signal, and/or fluorescence or sequence signal intensity. In certain embodiments, all of the primary nanostructures are the same or at least have the same spectral profile, sequencing signal, and/or fluorescence or sequence signal intensity. Conversely, it is understood that two or more of the primary nanostructures can be different. In certain embodiments, at least one primary nanostructure is linked to one or more secondary nanostructures. In certain embodiments, at least one nanostructure of the complex is a fluorescent nanostructure and/or comprises a unique identifying sequence.

Provided for herein are multiplexed compositions comprising two or more different primary nanostructures, at least one of which is part of a nanostructure complex according to any of the embodiments disclosed herein. In certain embodiments, at least one primary nanostructure comprises one or a combination of fluorophore moieties that give the primary nanostructure a spectral profile and the different primary nanostructures differ at least in the presence of fluorophore moieties, number of fluorophore moieties, position of the fluorophore moieties, and/or spectral profile of the fluorophore moieties. In certain embodiments, at least one primary nanostructure comprises a unique identifying sequence and the different primary nanostructures differ at least in the presence, number, and/or sequence of the unique identifying sequence.

In certain embodiments, at least two of the different primary nanostructures are conjugated to different specificity determining molecules. In certain embodiments, each of the different primary nanostructures is conjugated to a different specificity determining molecule. In certain embodiments, the different primary nanostructures differ only by the different specificity determining molecules conjugated to them. In certain embodiments, the different specificity determining molecules target different targets.

In certain embodiments, at least two of the different primary nanostructures of the multiplexed composition differ at least by the sequence of one or more nucleic acids comprising the primary nanostructure and/or at least two of the different primary nanostructures of the composition each comprise one or more at least partially single-stranded nucleic acid linker extensions and differ at least by the sequence and/or combination of their one or more at least partially single-stranded nucleic acid linker extensions. In certain embodiments, the at least two different primary nanostructures comprise the same spectral profile, the same sequence signal, and/or the same fluorescence intensity and/or sequence signal intensity.

In certain embodiments, the multiplexed composition comprises at least one primary nanostructure that is not linked to a secondary nanostructure. In certain embodiments, the multiplexed composition comprises two or more different nanostructure complexes. In certain embodiments, all of the primary nanostructures are part of a nanostructure complex.

In certain embodiments, at least two of the different nanostructure complexes having different primary nanostructures comprise different secondary nanostructures. In certain embodiments, the different nanostructure complexes having different primary nanostructures each comprise different secondary nanostructures. In certain embodiments, at least two of the different nanostructure complexes having different primary nanostructures comprise the same secondary nanostructure or least secondary nanostructures having the same spectral profile, sequencing signal, and/or fluorescence or sequence signal intensity. In certain embodiments, at least one secondary nanostructure comprises one or more fluorophore moieties that give the secondary nanostructure a spectral profile and the different secondary nanostructures differ at least in the presence of fluorophore moieties, number of fluorophore moieties, position of the fluorophore moieties, and/or spectral profile of the fluorophore moieties. In certain embodiments, at least one secondary nanostructure comprises a unique identifying sequence and the different secondary nanostructures differ at least in the presence, number, and/or sequence of unique identifying sequence.

In certain embodiments, at least two of the different secondary nanostructures differ at least by the sequence of one or more nucleic acids comprising the secondary nanostructure and/or at least two of the different secondary nanostructures each comprise an at least partially single-stranded nucleic acid linker extension and differ at least by the hybridizing region sequence of their at least partially single-stranded nucleic acid linker extensions. In certain embodiments, the at least two different secondary nanostructures comprise the same spectral profile, sequencing signal, and/or fluorescence or sequence signal intensity. In certain embodiments for each nanostructure complex, the primary nanostructure and each of its linked secondary nanostructures are the same or at least comprise the same spectral profile, sequencing signal, and/or fluorescence or sequence signal intensity. In certain embodiments, for each nanostructure complex, the primary nanostructure and each of its linked secondary nanostructures comprise the same number of fluorophore moieties and/or same number of unique identifying sequences.

In certain embodiments, for at least one nanostructure complex, the primary nanostructure comprises one or a combination of fluorophore moieties and is linked to a secondary nanostructure comprising one or more quenchers that absorb the fluorescence of and/or undergo Forster resonance energy transfer with the fluorophore moieties of the primary nanostructure.

In certain embodiments, the multiplexed composition comprises at least two nanostructure complexes and (i) the primary nanostructure of a first nanostructure complex comprises one or a combination of fluorophore moieties that give the primary nanostructure a spectral profile, wherein the primary nanostructure of said first nanostructure complex is linked to a secondary nanostructure comprising one or a combination of fluorophore moieties that give the secondary nanostructure a different spectral profile from its linked primary nanostructure and/or that give the first nanostructure complex a different spectral profile from its primary nanostructure; and (ii) the primary nanostructure of an at least second nanostructure complex comprises one or a combination of fluorophore moieties that give the primary nanostructure a different spectral profile than the spectral profile of the primary nanostructure of the first nanostructure complex, wherein the primary nanostructure of the at least second nanostructure complex is also linked to a secondary nanostructure comprising one or a combination of fluorophore moieties that give the secondary nanostructure a different spectral profile from its linked primary nanostructure and/or that give the second nanostructure complex a different spectral profile from its primary nanostructure. In certain embodiments, the spectral profile of at least one of the nanostructure complexes is different from the spectral profile of its primary nanostructure. In certain embodiments, the spectral profiles of at least two of the nanostructure complexes is different from the spectral profiles of their primary nanostructures. In certain embodiments, the spectral profiles of all of the nanostructure complexes is different from the spectral profiles of their primary nanostructures. In certain embodiments, at least two or all of the nanostructure complexes have spectral profiles. And, in certain embodiments, all of the nanostructure complexes have the same spectral profile.

In certain embodiments, the multiplexed composition comprises at least two nanostructure complexes wherein the primary nanostructure of a first complex comprises one or a combination of fluorophore moieties that give the primary nanostructure a spectral profile and the primary nanostructure of an at least second complex also comprises one or a combination of fluorophore moieties that give the primary nanostructure a spectral profile that is different from the spectral profile of the primary nanostructure of the first nanostructure complex, and wherein the secondary nanostructure linked to the primary nanostructure of the first nanostructure complex comprises one or a combination of fluorophore moieties that give it a spectral profile and the secondary nanostructure linked to the primary nanostructure of the at least second nanostructure complex comprises the same one or combination of fluorophore moieties as the secondary nanostructure linked to the primary nanostructure of the first nanostructure complex. In certain embodiments, the secondary nanostructure linked to the primary nanostructure of the first nanostructure complex and the secondary nanostructure linked to the primary nanostructure of the second nanostructure complex are the same.

In certain embodiments, at least two nanostructure complexes are fluorescent nanostructure complexes and differ from each other at least by their total number of fluorophore moieties and/or by their intensity of fluorescent signal. In certain embodiments, the nanostructure complexes also differ from each other by their spectral profiles. In certain embodiments, the number of fluorophore moieties of the primary nanostructure of one nanostructure complex differs from the number of fluorophore moieties of another primary nanostructure complex in the composition. In certain embodiments, the number of fluorophore moieties of a secondary nanostructure of one nanostructure complex differs from the number of fluorophore moieties of another secondary nanostructure complex in the composition.

The nanostructure complexes of the multiplexed compositions of this disclosure can comprise polymerized nanostructures as described in detail elsewhere herein. In certain embodiments, at least one nanostructure complex comprises a primary nanostructure linked to two or more secondary nanostructures. In certain embodiments, two or more nanostructure complexes comprise a primary nanostructure linked to two or more secondary nanostructures. In certain embodiments, at least one nanostructure complex comprising a primary nanostructure linked to two or more secondary nanostructures has a different number of secondary nanostructures than at least one other nanostructure complex comprising a primary nanostructure linked to two or more secondary nanostructures.

In certain embodiments, at least two nanostructure complexes comprise a unique identifying sequence and differ from each other at least by their total number of unique identifying sequences and/or by the intensity of their sequencing signal. In certain embodiments, the nanostructure complexes also differ from each other by the sequence of their unique identifying sequences. In certain embodiments, at least two nanostructure complexes target the same target molecule and at least one of said at least two nanostructure complexes comprises a unique identifying sequence and at least one other of said at least two nanostructure complexes does not comprise a unique identifying sequence with the same sequence or does not comprise a unique identifying sequence. In certain embodiments, the number of unique identifying sequences of a primary nanostructure of one nanostructure complex differs from the number of unique identifying sequences of another primary nanostructure of another nanostructure complex in the composition. In certain embodiments, the number of unique identifying sequences of a secondary nanostructure of one nanostructure complex differs from the number of unique identifying sequences of another secondary nanostructure of another nanostructure complex in the composition.

In certain embodiments, a multiplexed composition comprises at least two nanostructure complexes and at least two of the nanostructure complexes comprise a primary nanostructure linked to one or a combination of secondary nanostructures and further wherein one or combination of secondary nanostructures of one nanostructure complex is different from the one or a combination of secondary nanostructures of another nanostructure in the composition. In certain embodiments, at least two nanostructure complexes comprise in their one or a combination of secondary nanostructures at least one secondary nanostructure in common. In certain embodiments, at least two nanostructure complexes comprise in their one or combination of secondary nanostructures no secondary nanostructures in common.

In certain embodiments, one nanostructure complex has a different spectral profile and/or different intensity of fluorescent signal than that of at least one other nanostructure complex in the composition. In certain embodiments, one nanostructure complex has a different sequencing signal and/or different intensity of sequencing signal than that of at least one other nanostructure complex in the composition. In certain embodiments, at least one nanostructure complex has a unique fluorescence identity and/or sequencing identity to at least one other nanostructure complex and/or from any other nanostructure complex in the composition.

In certain embodiments, the secondary nanostructure or combination of secondary nanostructures linked to a primary nanostructure is determined by sequence complementarity of their at least partially single-stranded linker extensions with the sequence of the at least partially single-stranded linker extensions of the primary nanostructure. In certain embodiments, the sequence of the at least partially single-stranded linker extensions of one primary nanostructure is distinct for purposes of hybridization from the sequence of the at least partially single-stranded linker extensions of at least one other primary nanostructure in the composition.

It is understood that for any nanostructure complex or a nanostructure of the multiplexed composition of this disclosure, in certain embodiments at least one nanostructure complex is disclosed as bound to a target.

Provided for herein are methods of labeling a target molecule. In certain embodiments the methods comprise binding a nucleic acid nanostructure complex as described anywhere herein to the target molecule. In certain embodiments, the nanostructure complex is a part of a multiplexed composition described herein. In certain embodiments, the method further comprises measuring a fluorescent signal and/or sequencing signal of the labeled target molecule. In certain embodiments, the method further comprises detecting a labeled target molecule.

One of ordinary skill in the art will recognize that there are numerous different orders in which the specificity determining molecule and nanostructure complexes in varying degrees of assembly can be bound to a target. Several representative, non-limiting examples are provided herein.

In certain embodiments, the primary nanostructure binds to the target. For example, in certain embodiments, the method comprises (i) first attaching a primary nucleic acid nanostructure to the target molecule, wherein the primary nucleic acid nanostructure specifically binds to the target molecule and (ii) then attaching one or more secondary nanostructures to the primary nanostructure bound to the target molecule to form a nanostructure complex bound to the target molecule. In other embodiments, one or more secondary nanostructures, or all of the secondary nanostructures of the nanostructure complex, or all of the secondary nanostructures of the nanostructure complex except for one or more terminal nanostructures, can be bound to the primary nanostructure before the primary nanostructure is bound to the target molecule. As discussed in greater detail elsewhere herein, in certain embodiments, the one or more secondary nanostructures is attached to the primary nanostructure via hybridization between an at least partially single-stranded linker extension of the secondary nanostructure and an at least partially single-stranded linker extension of the primary nanostructure to form a hybridized at least partially double-stranded nucleic acid linker.

Certain embodiments comprise (i) first attaching a specificity determining molecule to the target molecule, wherein the specificity determining molecule specifically binds to the target molecule; (ii) next attaching a primary nanostructure to the specificity determining molecule bound to the target molecule; and (iii) then attaching one or more secondary nanostructures to the primary nanostructure bound to the specificity determining molecule, thus forming a nanostructure complex bound to the target molecule. In certain embodiments, the primary nanostructure is attached to the specificity determining molecule via hybridization between an at least partially single-stranded linker extension of the primary nanostructure and an at least partially single-stranded linker extension of the specificity determining molecule to form a hybridized at least partially double-stranded nucleic acid linker. In certain embodiments, the primary nanostructure is attached to the specificity determining molecule via a biotin/biotin-binding protein complex. In certain embodiments, the one or more secondary nanostructures is attached to the primary nanostructure via hybridization between an at least partially single-stranded linker extension of the secondary nanostructure and an at least partially single-stranded linker extension of the primary nanostructure to form a hybridized at least partially double-stranded nucleic acid linker.

Certain embodiments further comprise attaching at least one additional secondary nanostructure to a proximal secondary nanostructure that is already attached to a primary nanostructure. In certain embodiments, the at least one additional secondary nanostructure is attached to the proximal secondary nanostructure via hybridization between an at least partially single-stranded linker extension of the additional secondary nanostructure and an at least partially single-stranded linker extension of the proximal secondary nanostructure to form a hybridized at least partially double-stranded nucleic acid linker. Further, certain embodiments further comprise attaching at least one further additional secondary nanostructure to a secondary nanostructure that is already attached to a secondary nanostructure attached to a proximal secondary nanostructure, either adjacently or through intervening secondary nanostructures. In certain embodiments, the at least one further additional secondary nanostructure is attached to another secondary nanostructure via hybridization between an at least partially single-stranded linker extension of the further additional secondary nanostructure and an at least partially single-stranded linker extension of the other secondary nanostructure to form a hybridized at least partially double-stranded nucleic acid linker. In certain embodiments, the addition of the at least one further additional secondary nanostructure forms a linear polymer of secondary nanostructures attached to the primary nanostructure as described in greater detail elsewhere herein. In certain embodiments, the addition of two or more further additional secondary nanostructures forms a branched network of secondary nanostructures as described in greater detail elsewhere herein.

Certain components of a nanostructure can be pre-assembled before assembly of the final nanostructure. For example, in certain embodiments, two or more secondary nanostructures are linked together before they are incorporated into the nanostructure complex. Further, the order of assembly can be controlled even when various components are mixed together without regard to their order of incorporation. For example, in certain embodiments, two or more secondary nanostructures are simultaneously contacted with a primary nanostructure or a nanostructure complex that has already been partially assembled to comprise a primary nanostructure and at least one proximal secondary nanostructure, wherein the order of incorporation into the nanostructure complex of the two or more secondary nanostructures is determined by the sequence of an at least partially single-stranded nucleic acid linker extension of the secondary nanostructures (and not by the order by which they are added).

Further representative methods include wherein:
(i) the primary nanostructure and the specificity determining molecule comprising the nucleic acid nanostructure complex are already assembled together before binding the nanostructure complex to the target molecule;
(ii) the primary nanostructure and one or more secondary nanostructures comprising the nucleic acid nanostructure complex are already assembled together before binding the nanostructure complex to the target molecule;
(iii) the primary nanostructure and all secondary nanostructures comprising the nucleic acid nanostructure complex are assembled together before binding the nanostructure complex to the target molecule;
(iv) the primary nanostructure and one or more secondary nanostructures comprising the nucleic acid nanostructure complex are assembled together before attaching the primary nanostructure to the specificity determining molecule, after which the nanostructure complex is bound to the target molecule; and/or
(v) the primary nanostructure and all secondary nanostructures comprising the nucleic acid nanostructure complex are assembled together before attaching the primary nanostructure to the specificity determining molecule, after which the nanostructure complex is bound to the target molecule.

The terminal secondary nanostructure can serve a special purpose such as quenching the signal of the nanostructure complex. Thus, in certain embodiments, the primary nanostructure and all secondary nanostructures comprising the nanostructure complex are assembled together, except for one or more final terminal secondary nanostructures, before attaching the primary nanostructure to the specificity determining molecule and/or before binding the nucleic acid nanostructure complex to the target molecule. In certain embodiments, none of the final terminal secondary nanostructures are assembled onto the nanostructure complex before binding to the target molecule. Certain embodiments comprise attaching the final terminal secondary nanostructures following binding the rest of the assembled nanostructure complex to the target molecule. In certain embodiments, a fluorescent signal is measured before and after attaching one or more terminal secondary nanostructures.

In certain embodiments, at least the primary nanostructure and/or at least one secondary nanostructure of the nanostructure complex bound to the target molecule is a fluorescent nanostructure and/or comprises a unique identifying sequence. In certain embodiments, the method comprises measuring for a fluorescent signal and/or sequence signal from the labeled target molecule. In certain embodiments, a fluorescent signal and/or sequencing signal is detected. In certain embodiments the method comprises measuring for a fluorescent signal and/or sequencing signal after the primary nanostructure and/or nanostructure complex is bound to the target molecule but before the nanostructure complex is completely assembled and then measuring for a fluorescent signal and/or sequencing signal at least one additional time after at least one secondary nanostructure or additional secondary nanostructure is assembled into the nanostructure complex. In certain embodiments the method comprises measuring for a fluorescent signal from the labeled target molecule before a final terminal secondary nanostructure is attached and then measuring for a fluorescent signal after the final terminal secondary nanostructure is attached. In certain embodiments, the final terminal secondary nanostructure comprises one or more quenchers that absorb the fluorescence of and/or undergo Forster resonance energy transfer with a fluorophore moiety.

Provided for herein are multiplex methods of labeling one or more target molecules. In certain embodiments the methods comprise binding two or more nanostructure complexes of this disclosure or at least one nanostructure complex of this disclosure and at least one primary nanostructure to the one or more target molecules according the methods described above.

In certain embodiments at least two of the nanostructure complexes bind to the same target molecule but the nanostructure complexes differ in at least spectral profile, fluorescent intensity, sequencing signal, and/or sequencing signal intensity. In certain embodiments at least two of the nanostructure complexes bind to different target molecules but otherwise the nanostructure complexes have the same spectral profile, fluorescent intensity, sequencing signal, and/or sequencing signal intensity. In certain embodiments, at least two of the nanostructure complexes bind to different target molecules and the nanostructure complexes differ in at least spectral profile, fluorescent intensity, sequencing signal, and/or sequencing signal intensity. In certain embodiments, different nanostructure complexes differ at least in their primary nanostructures and/or wherein different nanostructure complexes differ at least in secondary nanostructures and/or number of secondary nanostructures. In certain embodiments, at least two different target molecules are bound by the same nanostructure complex or to nanostructure complexes having at least the same spectral profile, fluorescent signal intensity, sequencing signal, and/or sequencing signal intensity, and at least one other target molecule is bound to a nanostructure complex that differs in at least spectral profile, fluorescent signal intensity, sequencing signal, and/or sequencing signal intensity. In certain embodiments, each target molecule is bound to a different nanostructure complex that differs from the other nanostructure complex or complexes at least in spectral profile, fluorescent intensity, sequencing signal, and/or sequencing signal intensity. In certain embodiments, at least one nanostructure complex and at least one primary nanostructure bind to the same target molecule. In certain other embodiments, at least one nanostructure complex and at least one primary nanostructure bind to different target molecules.

In certain embodiments, for at least one nanostructure complex, the addition of one or more secondary nanostructures—either to form an at least partially assembled nanostructure complex before binding to a target molecule and/or to assemble a nanostructure complex wherein at least a portion of the nanostructure complex has already been bound to a target molecule—amplifies the fluorescence signal and/or sequencing signal in comparison to the primary nanostructure alone. In certain embodiments, the amplification of the signal can be stoichiometrically controlled. In certain embodiments, for at least one nanostructure complex, the primary nanostructure comprises one or a combination of fluorophore moieties that give the primary nanostructure a spectral profile and is linked to n number of secondary nanostructures having the same spectral profile, wherein the secondary nanostructures with the same spectral profile amplify the fluorescence signal of the nanostructure complex in comparison to the fluorescence signal of the primary nanostructure alone. In certain embodiments, the secondary nanostructures having the same spectral profile as the primary nanostructure also each comprise the same number of fluorophore moieties as the primary nanostructure and amplify the fluorescence signal of the nanostructure complex by a factor of about n+0.5, n+0.6, n+0.7, n+0.8, n+0.9 or n+1 times the amount of the fluorescence signal of the primary nanostructure alone. In certain embodiments the above is applied for at least two nanostructure complexes in a multiplex method.

In certain embodiments, for at least one nanostructure complex, the primary nanostructure comprises a unique identifying sequence and is linked to n number of secondary nanostructures comprising the same unique identifying sequence, wherein the secondary nanostructures with the same unique identifying sequence amplify the sequencing signal of the nanostructure complex in comparison to the sequencing signal of primary nanostructure alone. In certain embodiments, the secondary nanostructures also each comprise the same number of unique identifying sequences as the primary nanostructure and amplify the sequencing signal of the nanostructure complex by a factor of about n+0.5, n+0.6, n+0.7, n+0.8, n+0.9 or n+1 times the amount of the sequencing signal of the primary nanostructure alone. In certain embodiments the above is applied for at least two nanostructure complexes in a multiplex method.

In certain embodiments, for at least one nanostructure complex, the primary nanostructure comprises one or a combination of fluorophore moieties and is linked to a secondary nanostructure comprising one or more quenchers that absorb the fluorescence of and/or undergo Forster resonance energy transfer with the fluorophore moieties of the primary nanostructure. In certain embodiments the above is applied for at least two nanostructure complexes in a multiplex method.

In certain embodiments, for at least one nanostructure complex, the primary nanostructure comprises one or a combination of fluorophore moieties that give the primary nanostructure a spectral profile and is linked to a secondary nanostructure comprising a different fluorophore moiety or different combination of fluorophore moieties that give the secondary nanostructure and/or the nanostructure complex a spectral profile that is different from the primary nanostructure spectral profile. In certain embodiments, the spectral profile of the nanostructure complex is different from the spectral profile of the primary nanostructure. In certain embodiments the secondary nanostructure comprises a fluorophore moiety or combination of fluorophore moieties that can undergo Forster resonance energy transfer with the fluorophore moiety or combination of fluorophore moieties of the primary nanostructure. In certain embodiments, the fluorescence of the labeled target molecule is measured before and after the addition of a secondary nanostructure that gives the nanostructure complex a different spectral profile. In certain embodiments, the primary nanostructure is linked to at least two secondary nanostructures each comprising a different fluorophore moiety or a different combination of fluorophore moieties that give each secondary nanostructure and/or the nanostructure complex a spectral profile that is different from the primary nanostructure spectral profile. In certain embodiments, at least one of the linked secondary nanostructures comprises a different fluorophore moiety or different combination of fluorophore moieties that give such secondary nanostructure a spectral profile that is different from another linked secondary nanostructure spectral profile. In certain embodiments, each of the linked secondary nanostructures comprises a different fluorophore moiety or different combination of fluorophore moieties that give each secondary nanostructure a spectral profile that is different from any other linked secondary nanostructure spectral profile. Further, in certain embodiments the above is applied for at least two nanostructure complexes in a multiplex method.

In certain embodiments, for at least one nanostructure complex, the primary nanostructure comprises x number of a fluorophore moiety or a combination of fluorophore moieties and is linked to y number of secondary nanostructures each comprising z number of the same fluorophore moiety or combination of fluorophore moieties as the primary nanostructure, wherein z is independently determined for each secondary nanostructure and the sum of z from the secondary nanostructures is zₜₒₜₐₗ. The fluorescent signal of the nanostructure complex compared to the fluorescent signal of the primary nanostructure alone is amplified by a factor of about (x+zₜₒₜₐₗ)/x. In certain embodiments, z is the same for each secondary nanostructure and zₜₒₜₐₗ=y*z. In certain embodiments the above is applied for at least two nanostructure complexes in a multiplex method.

Further, once labeled by a method of this disclosure, a target molecule can be detected. Certain embodiments comprise labeling a target molecule according to the method of this disclosure and measuring for a fluorescent signal and/or sequence signal from the labeled target molecule. In certain embodiments, the method is a multiplex method and the method comprises labeling at least two target molecules and measuring for a fluorescent signal and/or sequence signal from the labeled target molecules.

Also provided for herein are kits for performing any method of this disclosure. In certain embodiments, a kit comprises a nanostructure complex described anywhere herein or a component thereof. In certain embodiments, a kit comprises reagents and/or apparatus for labeling a target molecule according to any method described herein. In certain embodiments, the kit further comprises instructions either printed and/or on an electronic storage medium, buffers and/or additional reagents, and/or packaging materials.

### Nucleic Acid Nanostructure Fluorescent Labels.

Nucleic acid nanostructure fluorescent labels have been described in detail in WO/2018/231805, which is incorporated herein by reference in its entirety. Nucleic acid nanostructure fluorescent labels, which can be used as labels can be created via a variety of techniques. In some examples, DNA self-assembly can be used to ensure that the relative locations of the resonators within a label correspond to locations specified according to a desired temporal decay profile. For example, each resonator of the network could be coupled to a respective specified DNA strand. Each DNA strand could include one or more portions that complement portions one or more other DNA strands such that the DNA strands self-assemble into a nanostructure that maintains the resonators at the specified relative locations.

In certain embodiments the nucleic acid nanostructure fluorescent label comprises one or more polynucleotides. In certain embodiments one or more of those polynucleotides has a length of at least about 10, 15, 20, 25, 30, 35, 40, 50, 55, 60, 65, 75, 80, 85, 90, 95, 100, 110, 120, 130, 140, 150, 175, 200, 300, 400, 500, 750, 1000, 2000, 3000, 4000, 5000, 7500, or 10,000 nucleotides, or any range in between. In certain embodiments one or more of those polynucleotides has a length of at least about 10, 15, 20, 25, 30, 35, 40, 50, 55, 60, 65, 75, 80, 85, 90, 95, 100, 110, 120, 130, 140, 150, 175, 200, 300, or 400 nucleotides, or any range in between. In certain embodiments, one or more of those polynucleotides has a length of at least about 20, 25, 30, 35, 40, 50, 55, 60, 65, 75, 80, 85, 90, 95, 100, 110, or 120 nucleotides, or any range in between. In certain embodiments, the nucleic acid nanostructure fluorescent label comprises two, three, four, five, six or more polynucleotides. In certain embodiments, the nucleic acid nanostructure fluorescent label comprises a total number of nucleotides of at least about 50, 100, 200, 500, 1000, 5000, 10000, 15000, 20000, or any range in between.

DNA self-assembly and other emerging nano-scale manufacturing techniques permit the fabrication of many instances of a specified structure with precision at the nano-scale. For example, as described in WO/2018/231805, a nucleic acid nanostructure is made by annealing custom, synthetic DNA produced by chemical methods. The multiple strands are pre-conjugated to fluorophores, peptides, small molecules, etc. prior to being mixed and annealed. The sequences are designed such that there is a single, finite assembly of lowest energy and is stable in solution, dry, or frozen and preserves the relative location of any conjugated materials. Such precision can permit fluorophores, quantum dots, dye molecules, plasmonic nanorods, or other optical resonators to be positioned at precise locations and/or orientations relative to each other in order to create a variety of optical resonator networks. Such resonator networks may be specified to facilitate a variety of different applications. In some examples, the resonator networks could be designed such that they exhibit a pre-specified temporal relationship between optical excitation (e.g., by a pulse of illumination) and re-emission; this could enable temporally-multiplexed labels and taggants that could be detected using a single excitation wavelength and a single detection wavelength. Additionally, or alternatively, the probabilistic nature of the timing of optical re-emission, relative to excitation, by these resonator networks could be leveraged to generate samples of a random variable. These resonator networks may include one or more “input resonators” that exhibit a dark state; resonator networks including such input resonators may be configured to implement logic gates or other structures to control the flow of excitons or other energy through the resonator network. Such structures could then be used, e.g., to permit the detection of a variety of different analytes by a single resonator network, to control a distribution of a random variable generated using the resonator network, to further multiplex a set of labels used to image a biological sample, or to facilitate some other application.

These resonator networks include networks of fluorophores, quantum dots, dyes, Raman dyes, conductive nanorods, chromophores, or other optical resonator structures. The networks can additionally include antibodies, aptamers, strands of deoxyribonucleic acid (DNA) or ribonucleic acid (RNA), or other receptors configured to permit selective binding to analytes of interest (e.g., to a surface protein, molecular epitope, characteristic nucleotide sequence, or other characteristic feature of an analyte of interest). The labels can be used to observe a sample, to identify contents of the sample (e.g., to identify cells, proteins, or other particles or substances within the sample), to sort such contents based on their identification (e.g., to sort cells within a flow cytometer according to identified cell type or other properties), or to facilitate some other applications. In certain embodiments disclosed herein the labels are linked to a substrate, such as an antibody or bead, via a polynucleotide linker.

In an example application, such resonator networks may be applied (e.g., by coupling the resonator network to an antibody, aptamer, or other analyte-specific receptor) to detect the presence of, discriminate between, or otherwise observe a large number of different labels in a biological or material sample or other environment of interest. Such labels may permit detection of the presence, amount, or location of one or more analytes of interest in a sample (e.g., in a channel of a flow cytometry apparatus). Having access to a large library of distinguishable labels can allow for the simultaneous detection of a large number of different analytes. Additionally, or alternatively, access to a large library of distinguishable labels can allow for more accurate detection of a particular analyte (e.g., a cell type or sub-type of interest) by using multiple labels to bind with the same analyte, e.g., to different epitopes, surface proteins, or other features of the analyte. Yet further, access to such a large library of labels may permit selection of labels according to the probable density or number of corresponding analytes of interest, e.g., to ensure that the effective brightness of different labels, corresponding to analytes having different concentrations in a sample, is approximately the same when optically interrogating such a sample.

Such labels may be distinguishable by virtue of differing with respect to an excitation spectrum, an emission spectrum, a fluorescence lifetime, a fluorescence intensity, a susceptibility to photobleaching, a fluorescence dependence on binding to an analyte or on some other environmental factor, a polarization of re-emitted light, or some other optical properties.

WO/2018/231805 describes methods for specifying, fabricating, detecting, and identifying optical labels that differ with respect to temporal decay profile and/or excitation and emission spectra. Additionally, or alternatively, the provided labels may have enhanced brightness relative to existing labels (e.g., fluorophore-based labels) and may have a configurable brightness to facilitate panel design or to permit the relative brightness of different labels to facilitate some other consideration. Such labels can differ with respect to the time-dependent probability of re-emission of light by the label subsequent to excitation of the label (e.g., by an ultra-fast laser pulse). Additionally, or alternatively, such labels can include networks of resonators to increase a difference between the excitation wavelength of the labels and the emission wavelength of the labels (e.g., by interposing a number of mediating resonators between an input resonator and an output resonator to permit excitons to be transmitted between input resonators and output resonators between which direct energy-transfer is disfavored). Yet further, such labels may include logic gates or other optically-controllable structures to permit further multiplexing when detecting and identifying the labels.

Resonator networks (e.g., resonator networks included as part of labels) as described in WO/2018/231805 can be fabricated in a variety of ways such that one or more input and/or readout resonators, output resonators, dark-state-exhibiting “logical input” resonators, and/or mediating resonators are arranged according to a specified network of resonators and further such that a temporal decay profile of the network, a brightness of the network, an excitation spectrum, an emission spectrum, a Stokes shift, or some other optical property of the network, or some other detectable property of interest of the network (e.g., a state of binding to an analyte of interest) corresponds to a specification thereof (e.g., to a specified temporal decay profile, a probability of emission in response to illumination). Such arrangement can include ensuring that a relative location, distance, orientation, or other relationship between the resonators (e.g., between pairs of the resonators) correspond to a specified location, distance, orientation, or other relationship between the resonators.

This can include using DNA self-assembly to fabricate a plurality of instances of one or more resonator networks. For example, a number of different DNA strands could be coupled (e.g., via a primary amino modifier group on thymidine to attach an N-Hydroxysuccinimide (NHS) ester-modified dye molecule) to respective resonators of a resonator networks (e.g., input resonators, output resonator, and/or mediator resonators). Pairs of the DNA strands could have portions that are at least partially complementary such that, when the DNA strands are mixed and exposed to specified conditions (e.g., a specified pH, or a specified temperature profile), the complementary portions of the DNA strands align and bind together to form a semi-rigid nanostructure that maintains the relative locations and/or orientations of the resonators of the resonator networks.

In a representative resonator network, an input resonator, an output resonator and two mediator resonators are coupled to respective DNA strands. The coupled DNA strands, along with additional DNA strands, then self-assemble into the illustrated nanostructure such that the input resonator, mediator resonators, and output resonator form a resonator wire. In some examples, a plurality of separate identical or different networks could be formed, via such methods or other techniques, as part of a single instance of a resonator network (e.g., to increase a brightness of the resonator network).

The distance between resonators of such a resonator network could be specified such that the resonator network exhibits one or more desired behaviors (e.g., is excited by light at a particular excitation wavelength and responsively re-emits light at an emission wavelength according to a specified temporal decay profile). This can include specifying the distances between neighboring resonators such that they are able to transmit energy between each other (e.g., bidirectionally or unidirectionally) and further such that the resonators do not quench each other or otherwise interfere with the optical properties of each other. In examples wherein the resonators are bound to a backbone via linkers (e.g., to a DNA backbone via an amide bond (created, e.g., by N-Hydroxysuccinimide (NHS) ester molecules) or other linking structures), the linkers can be coupled to locations on the backbone that are specified with these considerations, as well as the length(s) of the linkers, in mind. For example, the coupling locations could be separated by a distance that is more than twice the linker length (e.g., to prevent the resonators from coming into contact with each other, and thus quenching each other or otherwise interfering with the optical properties of each other). Additionally, or alternatively, the coupling locations could be separated by a distance that is less than a maximum distance over which the resonators may transmit energy between each other. For example, the resonators could be fluorophores or some other optical resonator that is characterized by a Forster radius when transmitting energy via Forster resonance energy transfer, and the coupling locations could be separated by a distance that is less than the Forster radius.

The following examples are offered by way of illustration and not by way of limitation.

### EXAMPLES

### Example 1. Effects of Single Stranded DNA (ssDNA) Linker Sequence on Nucleic Acid Nanostructure Fluorescence

The purpose of this study was to examine whether the sequence of the ssDNA linker extension of a nucleic acid nanostructure affects its fluorescence properties.

Materials and Methods: Three nucleic acid nanostructures, each with different spectral properties and underlying fluorophore compositions, were tested in this experiment. For each nanostructure, two different versions were assembled: one containing a polyT ssDNA linker extension and one containing a ssDNA linker of the same length but composed of a mixture of bases. Each nanostructure was assembled using the same standard procedure. After assembly, the fluorescence spectrum of each nanostructure was measured in a 96-well plate using a VARIOSKAN LUX microplate reader (Thermo Fisher Scientific, Waltham, MA).

Results: FIG. 3 shows the overlaid fluorescence spectra for all three nucleic acid nanostructures assembled with either the polyT linker or the linker composed of a mixture of bases. The nearly identical spectral overlays show that that the sequence of the linker does not have a significant impact on the fluorescence spectrum or quantum yield of the nanostructure. Since the shape of the spectra is formed as a function of the assembly process, i.e., by arranging multiple fluorophores into interacting FRET networks, these results also provide supporting evidence that the assembly yield was not significantly altered by the sequence of the ssDNA linker extension. If the yield were affected, one would expect to see a change in the spectrum due to unincorporated fluorophore that cannot participate in the FRET process. Furthermore, by testing three nanostructures, each with different spectral properties, this experiment illustrates that these results are generalizable across different underlying fluorophore compositions as long as they are assembled using the same nucleic acid scaffold.

### Example 2. Experimental Methods of Fluorescence Amplification Using Primary-Secondary NOVAFLUOR Yellow 610 Nucleic Acid Nanostructure Complexes

This example was performed to prove that the dimer structure P-(L-S)₁, as described herein, formed and enabled an amplified fluorescence signal relative to a monomer control. Flow cytometry and size exclusion chromatography (SEC) experiments were performed to provide evidence that the dimer formed and amplified the fluorescence signal.

Dimers were formed by including a linker extension in each NOVAFLUOR Yellow 610 nucleic acid nanostructure that contains at least partially complementary regions (as described in FIG. 1). Two different length linker extensions were tested. One linker extension was 24 nucleotides long (“Chain-1”) and the other was 32 nucleotides long (“Chain-2”). The dimers that formed with these linker extensions were called Dimer Chain-1 and Dimer Chain-2, respectively. A chain refers to the at least partially double-stranded structure formed between the partially complementary regions of the primary and secondary nucleic acid nanostructures.

Sample Preparation: One-to-one molar equivalents of primary and secondary nucleic acid nanostructures (NOVAFLUOR Yellow 610, Thermo Fisher Scientific, Waltham, MA) were mixed to form the dimer structure of P-(L-S)₁. Once dimers were formed, they were either analyzed by size exclusion chromatography (SEC) or conjugated to anti-human CD4 antibodies (Clone SK3) for staining. The CD4 stains were prepared by mixing the dimer with a single-stranded DNA modified anti-CD4 antibody in a one-to-one molar ratio. A monomer control stain was prepared in the same way.

Flow Cytometry Analysis: Peripheral blood mononuclear cells (PBMCs) were first treated with a solution of CELLBLOX Monocyte and Macrophage Blocking Buffer (Thermo Fisher Scientific) to prevent non-specific binding of the antibody or nucleic acid nanostructure to the cell surface. Next, the cells were stained with either the monomer, Dimer Chain-1, or Dimer Chain-2 conjugates. An unstained cell sample was prepared as well. The stained cells were analyzed on an ATTUNE NxT flow cytometer (Thermo Fisher Scientific). FCS files were processed with FLOWJO software (BD Biosciences).

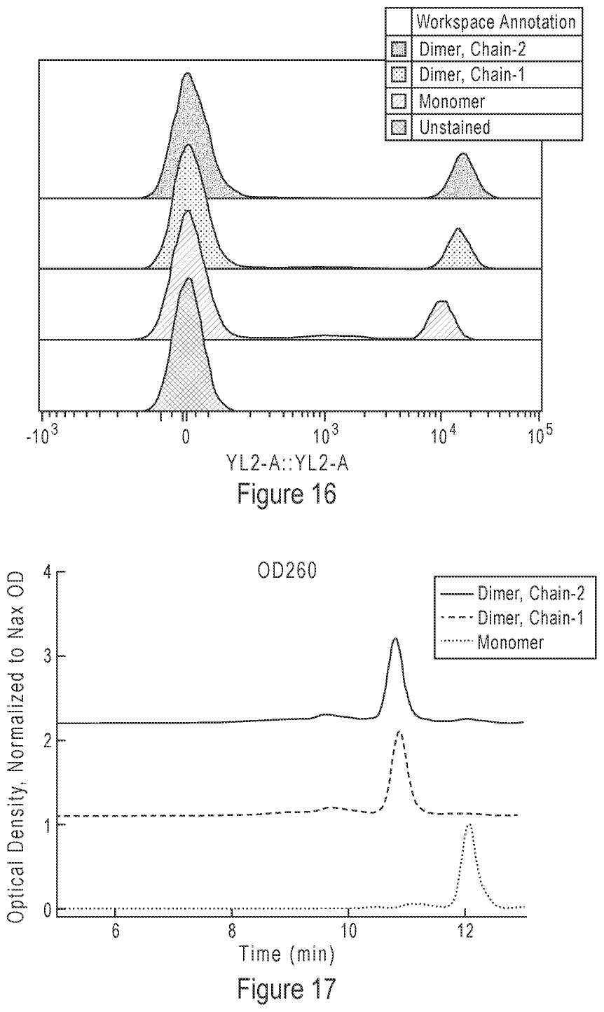

In the flow cytometry experiment, an amplified signal relative to the monomer control was expected to be observed. As seen in FIG. 16 and Table 1 below, the signals from Dimer Chain-1 and Chain-2 were brighter than the monomer by a factor of 1.5 and 1.7, respectively.

TABLE 1
Positive median fluorescence intensity (MFI+) in the YL2 detector
channel from monomer and dimer NOVAFLUOR Yellow
610 anti-CD4 antibody conjugate stained PBMCs. The x Factor
indicates signal intensity increase relative to the monomer.

| Sample Name | MFI+ | x Factor, MFI |
|---|---|---|
| Dimer, Chain-2 | 16,742 | 1.67 |
| Dimer, Chain-1 | 14,860 | 1.48 |
| Monomer | 10,024 | 1.00 |

Size Exclusion Chromatography Analysis: For the SEC experiment, the monomer and dimer NOVAFLUOR Yellow 610 nucleic acid nanostructures were injected onto an SEC column. The optical density was monitored at 260 nm, corresponding to DNA absorption. Data were processed with Octave software version 4.4.1 (John W. Eaton, David Bateman, Soren Hauberg, Rik Wehbring (2018). GNU Octave version 4.4.1 manual: a high-level interactive language for numerical computations, at the world-wide web at https://www.gnu.org/software/octave/doc/v4.4.1/).

In the SEC experiment, the larger dimer structure was expected to elute before the smaller monomer structure. FIG. 17 shows that the dimer structures (Chain-1 and Chain-2) eluted earlier than the monomer. These results indicate that the dimer is a larger structure than the monomer, thus providing evidence of dimer formation.

The above described flow cytometry and SEC experiments provided substantial evidence of dimer formation and resulted in amplification of the fluorescence signal. NOVAFLUOR Yellow 610 is one of many such nucleic acid nanostructures, all sharing similar molecular properties. Thus, it is contemplated that many nucleic acid nanostructures will behave in a manner similar to NOVAFLUOR Yellow 610, exhibiting an amplified signal in flow cytometry and shorter retention times in SEC than the respective monomer.

### Example 3. Methods of Fluorescence Amplification and Multicolor Barcoding Using Two-Step Primary and Secondary Nucleic Acid Nanostructure Staining

This illustrative example is to demonstrate that the dimer structure P-(L-S)₁, as described herein, will form using two separate staining steps. In the first step, cell staining occurs with only the primary nucleic acid nanostructure-antibody conjugate. In the second step, a secondary nucleic acid nanostructure binds to the primary nucleic acid nanostructure and forms a dimer. The fluorescence of the dimer can then be measured.

Dimers form because the primary and secondary nucleic acid nanostructures have linker extensions that are at least partially complementary to each other. In some embodiments, the dye compositions of the primary and secondary nucleic acid nanostructures are the same and thus the dimer will amplify the fluorescence signal relative to the primary nucleic acid nanostructure-antibody conjugate (see Example 1). In other embodiments the primary and secondary nucleic acid nanostructures will have different and unique spectral profiles, for example, to enable multicolor barcoding.

Cell Preparation: Peripheral blood mononuclear cells (PBMCs) are first treated with a solution of CELLBLOX Monocyte and Macrophage Blocking Buffer (Thermo Fisher Scientific) to prevent non-specific binding of the antibody or nucleic acid nanostructure (e.g., a NOVAFLUOR nanostructure) to the cell surface. Next, the cells are stained with the primary nucleic acid nanostructure-antibody conjugates. An unstained cell sample is prepared as well. Afterwards, the cells are washed to remove any unbound or non-specifically bound conjugates from the cells. Finally, the cells are split into two separate samples.

To one sample, the secondary nucleic acid nanostructure is added with the appropriate linker extension such that it can hybridize to the complementary linker on the primary nucleic acid nanostructure-antibody conjugate. To the other sample, a secondary nucleic acid nanostructure negative control is added that does not have the linker extension, and thus, should not hybridize to the primary nucleic acid nanostructure-antibody conjugate. This sample also acts as a negative control to show that secondary nucleic acid nanostructures do not have substantial non-specific binding to the primary nucleic acid nanostructure, antibody, or cells. Both cell samples are washed after staining to remove any unbound or non-specifically bound secondary nucleic acid nanostructure from the cells.

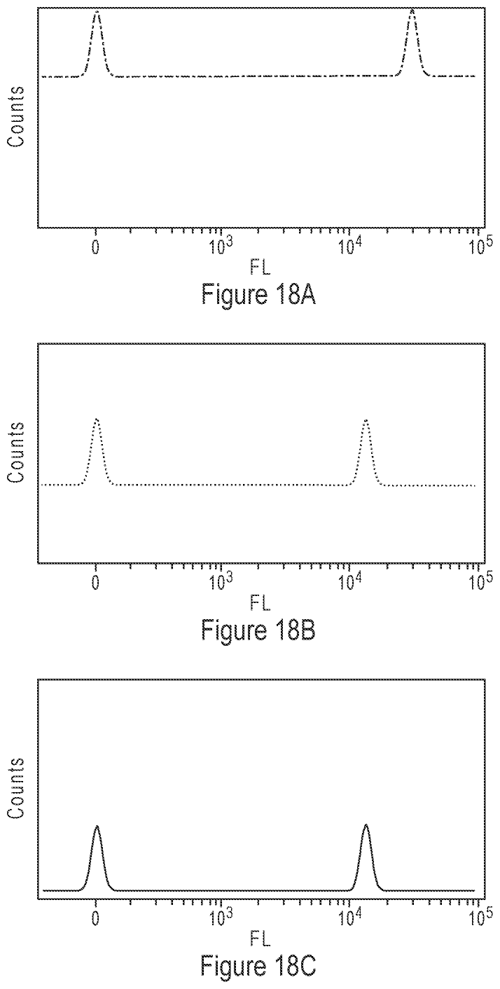

Signal Amplification when Primary and Secondary Nucleic Acid Nanostructures have the Same Dye Composition: When the cells are acquired by flow cytometry, the positive median fluorescence intensity (MFI+) of the amplified sample is expected to be greater than the primary nucleic acid nanostructure antibody-conjugate and the secondary nucleic acid nanostructure negative control samples. FIG. 18A and Table 2 show the predicted doubling of signal relative to FIG. 18B and FIG. 18C. Because the secondary nucleic acid nanostructure negative control does not comprise a linker extension, dimer formation is not expected upon adding the nucleic acid nanostructure. If this is true, then there will be no signal amplification. FIG. 18C and Table 2 show the prediction that adding a secondary nucleic acid nanostructure negative control to the primary nucleic acid nanostructure-antibody conjugate does not result in amplification of the signal. The significance of this result is that the secondary nucleic acid nanostructure does not bind to the primary nucleic acid nanostructure or cells non-specifically. Moreover, the result will demonstrate that the linker extension is required for the secondary nucleic acid nanostructure to bind, as seen in comparing FIG. 18A and FIG. 18C.

TABLE 2
Predicted positive MFI (MFI+) in the FL detector channel from
the Primary, Primary + Secondary, and Primary + Secondary
Negative Control nucleic acid nanostructure-antibody conjugates
of Example 2. The x Factor indicates signal intensity relative
to the primary nucleic acid nanostructure conjugate.

| Sample Name | MFI+ | x Factor, MFI |
|---|---|---|
| Primary + Secondary | 30,000 | 2.0 |
| Primary | 15,000 | 1.0 |
| Primary + Secondary Negative Control | 15,000 | 1.0 |

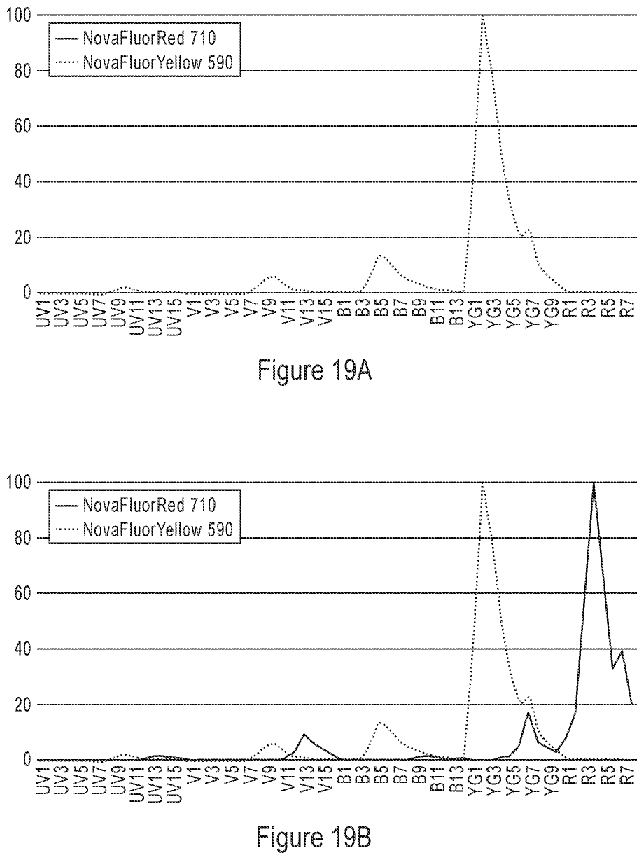

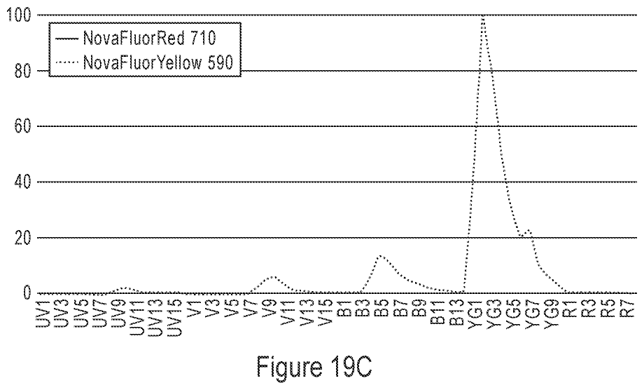

Multicolor Barcoding when Primary and Secondary Nucleic Acid Nanostructures have Different Dye Compositions: FIG. 19A represents how the primary nucleic acid nanostructure spectral signature will appear prior to adding the secondary nucleic acid nanostructure. Here, NOVAFLUOR Yellow 590 (Thermo Fisher Scientific) serves as an example spectral profile for the primary nucleic acid nanostructure and NOVAFLUOR Red 710 (Thermo Fisher Scientific) serves as an example secondary nucleic acid nanostructure. FIG. 19B shows the type of data to expect if NOVAFLUOR Red 710 is added to the primary nucleic acid nanostructure. Note that in this example, the individual spectral profiles are overlaid; however, when actually measured, a summed spectral profile will be acquired, where the resulting profile will depend on the individual contributing profiles' relative brightness from the two NOVAFLUOR nucleic acid nanostructures. FIG. 19C shows the type of data to expect if the secondary NOVAFLUOR Red 710 is a negative control (i.e. no linker extension). In this case, only the original NOVAFLUOR Yellow 590 signature will be observed, demonstrating the need for the linker extension to form a dimer. The exemplary data shown in FIG. 19A and FIG. 19B are indicative of a successful two-step approach to forming dimers and the ability to modify the fluorescence signature relative to the signal from the primary nucleic acid nanostructure-antibody conjugate. The exemplary data shown in FIG. 19C are indicative that the linker extension is a prerequisite for the secondary nucleic acid nanostructure to bind and that it has no observable non-specific binding effects on the spectral profile of primary nucleic acid nanostructure.

### Example 4. Fluorescence Amplification Using Streptavidin as an Intermediate Cross-Linker

The purpose of this study was to demonstrate amplification of antibody-nucleic acid nanostructure conjugate signal utilizing streptavidin as an intermediate cross-linker. The potential amplification possible using this method was demonstrated in flow cytometry experiments using NOVAFLUOR Yellow 610 (Thermo Fisher Scientific) and a dimer structure of NOVAFLUOR Yellow 610. It is contemplated that these reagents and method are generalizable to any nucleic acid nanostructure and antibody combination.

Three strategies were tested for utilizing streptavidin to amplify the fluorescence signal:
1. Streptavidin was complexed to NOVAFLUOR Yellow 610 using amine-reactive labeling chemistry, leaving open all biotin binding sites. This streptavidin-NOVAFLUOR Yellow 610 complex was used as a secondary stain for biotinylated antibodies in flow cytometry.
2. NOVAFLUOR Yellow 610 was biotinylated by incorporating a linker oligo modified at the 3′ terminus with biotin, then was complexed with streptavidin in various ratios. This streptavidin-NOVAFLUOR Yellow 610 complex was used as a secondary stain for biotinylated antibodies in flow cytometry.
3. NOVAFLUOR Yellow 610 was biotinylated by incorporating a linker oligo modified at the 3′ terminus with biotin, then was complexed with streptavidin in various ratios. This streptavidin-NOVAFLUOR Yellow 610 complex was then incubated with a biotinylated antibody to form an antibody-streptavidin-NOVAFLUOR Yellow 610 complex, which was used as a primary stain in flow cytometry.

In any of these methods, a nucleic acid nanostructure dimer can be used in place of a nucleic acid nanostructure monomer to increase the signal amplification. Proof of principle of this approach was demonstrated using Strategy 2. A schematic illustration of each strategy is shown in FIG. 20.

### Materials and Methods:

### Streptavidin Labeling Using Amine-Reactive Chemistry.

Purified streptavidin was labeled using a single-stranded linker oligo by targeting reactive amines on the protein, then was purified to remove the excess linker oligo and unlabeled streptavidin. The streptavidin-linker conjugate was incubated for 24 hours at 4° C. with a NOVAFLUOR nucleic acid nanostructure containing a complementary single-stranded linker to form the streptavidin-NOVAFLUOR nucleic acid nanostructure conjugate.

### Streptavidin Labeling Using Biotinylated Nucleic Acid Nanostructures.

To synthesize biotinylated NOVAFLUOR nucleic acid nanostructure, the NOVAFLUOR nucleic acid nanostructure was first folded with a short single-stranded DNA extension from one of the nanostructure's arms. The NOVAFLUOR nucleic acid nanostructure was then incubated for 24 hours at 4° C. with one equivalent of the complementary single-stranded DNA containing a 3′ biotin modification. To form the streptavidin-NOVAFLUOR nucleic acid nanostructure complex, purified streptavidin was incubated with 1 or 2 equivalents of biotinylated NOVAFLUOR nucleic acid nanostructure for 24 hours at 4° C.

### Flow Cytometry.

All staining and blocking steps were performed on ice using 1 million cells. Human peripheral blood mononuclear cells (PBMCs) were first treated with a solution of CELLBLOX Monocyte and Macrophage Blocking Buffer (Thermo Fisher Scientific) to prevent non-specific binding of the antibody or NOVAFLUOR nucleic acid nanostructure to the cell surface. The subsequent steps specific to each strategy tested were:
Strategy 1: The cells were stained with Biotin—anti-human CD4 antibody (Clone SK3), then washed twice to remove any excess biotinylated antibody. The cells were blocked again with CELLBLOX Monocyte and Macrophage Blocking Buffer, and then were stained with streptavidin-NOVAFLUOR Yellow 610 (labeled using amine-reactive chemistry).
Strategy 2: The cells were stained with Biotin—anti-human CD4 antibody (Clone SK3), then washed twice to remove any excess biotinylated antibody. The cells were blocked again with CELLBLOX Monocyte and Macrophage Blocking Buffer (Thermo Fisher Scientific), and then were stained with a pre-formed complex of streptavidin and NOVAFLUOR Yellow 610-Biotin at a ratio of 1:0, 1:1, or 1:2.
Strategy 3: The cells were stained with a pre-formed complex of Biotin-anti-human CD4 antibody (Clone SK3), streptavidin, and NOVAFLUOR Yellow 610-Biotin at a ratio of 1:1:2.

After staining was complete, the cells were washed once more. The cells were analyzed on an ATTUNE NxT flow cytometer (Thermo Fisher Scientific) and the FCS files were analyzed with FLOJO software (BD Biosciences).

### Results

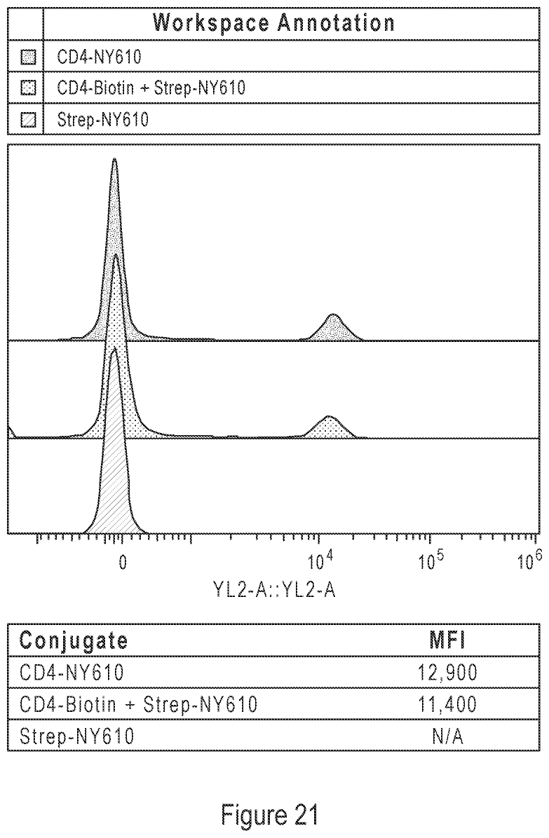

Three strategies were investigated to amplify the fluorescence signal in flow cytometry using streptavidin as a cross-linker. All of these strategies relied on a biotinylated primary antibody. Anti-human CD4 antibody (Clone SK3) was used in this example, but any other biotinylated antibody could be utilized. The first strategy left all biotin binding sites of the streptavidin tetramer open to binding. In flow, a two-step staining procedure was performed, first with the Biotin—anti-human CD4 antibody (Clone SK3) followed by the NOVAFLUOR modified streptavidin. The staining using this strategy was almost equal in performance to the primary anti-human CD4 antibody-NOVAFLUOR conjugate shown in FIG. 21, suggesting attachment of a single streptavidin-NOVAFLUOR complex to each Biotin-anti-human CD4 antibody.

The second and third strategies utilized the biotin binding sites for attaching NOVAFLUOR nucleic acid nanostructures. The NOVAFLUOR nucleic acid nanostructure was easily and quantitatively biotinylated by incorporating a 3′ biotinylated strand into the structure. The biotinylated nucleic acid nanostructure was then incubated at varying excess with purified streptavidin to form a streptavidin-NOVAFLUOR nucleic acid nanostructure conjugate. This approach formed a mixture of complexes. For example, for a 1:2 ratio of streptavidin:NOVAFLUOR nucleic acid nanostructure, the predominant complex formed was 1:2, but the other possible ratios also formed at lower concentrations. At higher ratios of NOVAFLUOR nucleic acid nanostructure, more biotin-binding sites were used up and fewer were left to bind to the biotinylated antibody. It was observed that above a 1:2 ratio of streptavidin:NOVAFLUOR nucleic acid nanostructure, the signal from the complex did not further increase, likely due to there being too few biotin-binding sites left for attachment to the antibody.

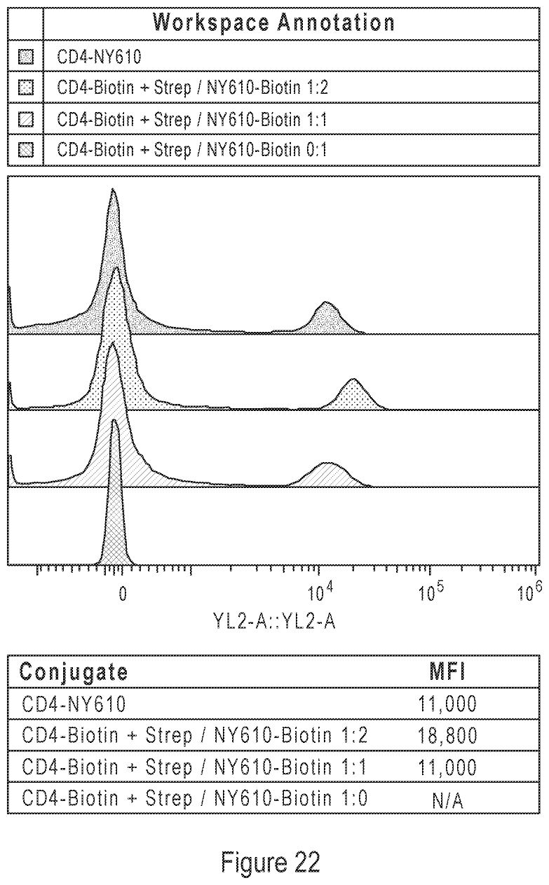

Using the second strategy and a 1:1 ratio of streptavidin:NOVAFLUOR nucleic acid nanostructure, an identical MFI compared to the primary CD4-NovaFluor conjugate was observed (FIG. 22). At a 2:1 ratio, the MFI increased by 1.64×, indicating amplification of the fluorescence signal.

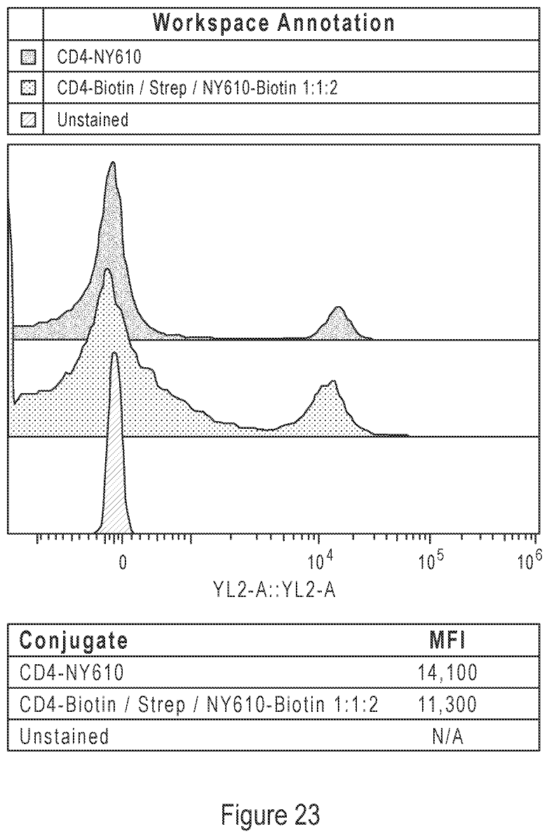

For Strategy 3, the 2:1 ratio was tested, but this complex was pre-incubated with one equivalent of the Biotin—anti-human CD4 antibody (Clone SK3). This enabled the entire antibody-streptavidin-NOVAFLUOR nucleic acid nanostructure complex to be used as a primary stain, simplifying the staining procedure in flow. Formed in this way, an increase in the fluorescence in flow cytometry experiments was not observed compared to the primary anti-CD4 antibody-NOVAFLUOR nucleic acid nanostructure conjugate (FIG. 23), as was predicted to be observed with the two-step staining using Strategy 2. Instead, the MFI of the complex was slightly lower, and the background staining increased substantially. Further optimization could reduce the background staining and increase the MFI of the complex to match that of the stain formed using two-step staining.

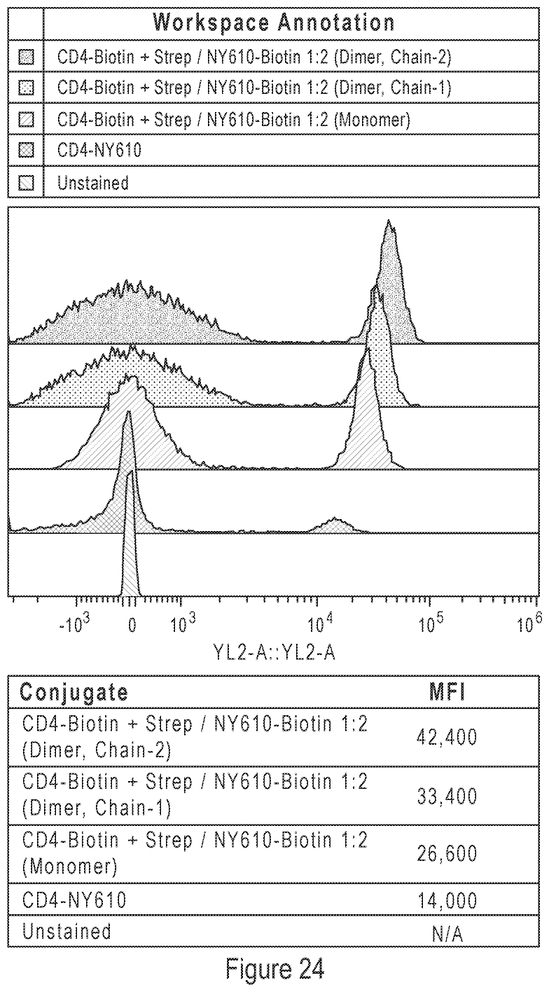

To further increase the fluorescence amplification factor, nucleic acid nanostructures (e.g., NOVAFLUOR nucleic acid nanostructure) can be chained together for any of the three strategies described above. In some embodiments, this occurs prior to attachment to streptavidin. To chain nucleic acid nanostructures, a secondary nucleic acid nanostructure is attached to the opposite arm of the primary nucleic acid nanostructure, which contains the linker for attachment to the antibody or streptavidin. These nucleic acid nanostructures are hybridized to one another through complementary single-stranded linker extensions that in this example form a 24- or 36-base pair linkage, referred to as Chain 1 and Chain 2, respectively. The chaining linker sequence is unique from the linker sequence targeting the antibody or streptavidin, giving orthogonal targeting capability. The nucleic acid nanostructure dimer is formed before attachment to streptavidin, which is demonstrated using Strategy 2 above. As shown in FIG. 24, using a 2:1 complex of NOVAFLUOR Yellow 610 monomer conjugated to streptavidin and staining cells labeled with Biotin—anti-human CD4 antibody (Clone SK3) is expected to result in a 1.9-fold increase in signal compared to the primary antibody directly labelled with NOVAFLUOR Yellow 610. By using nucleic acid nanostructure dimers instead, the amplification can be increased even further to a maximum of 3-fold over the primary antibody when using the Chain-2 dimer. A similar approach could be used to amplify fluorescence signals with strategies 1 or 3 as well.

### Example 5: Experimental Methods of Fluorescence Amplification Using Primary-Secondary NOVAFLUOR Ultraviolet Nucleic Acid Nanostructure Complexes

Overview: The purpose of the following study was to further demonstrate that the dimer structure P-(L-S)₁, as described herein, formed and enabled an amplified fluorescence signal, relative to a monomer control. Flow cytometry experiments were performed to provide evidence that the dimer formed and enabled amplification of the fluorescence signal.

Dimers were formed by including a linker extension in each nucleic acid nanostructure that contained at least partially complementary regions, as seen in FIG. 1. Herein, the dimer's ability to amplify the fluorescence signal was tested for the following nucleic acid nanostructures: NOVAFLUOR Ultraviolet 430, NOVAFLUOR Ultraviolet 445, and NOVAFLUOR Ultraviolet 755 (Thermo Fisher Scientific). The linker extension in these studies was 32 nucleotides long.

Sample Preparation: One-to-one molar equivalents of primary and secondary NOVAFLUOR Ultraviolet nanostructures were mixed to form the dimer structure of P-(L-S)₁. Monomer and Dimer nanostructures were conjugated to anti-human CD4 (Clone SK3) for staining. The CD4 stains were prepared by mixing the monomer or dimer with a single-stranded DNA modified CD4 antibody in a one-to-one molar ratio.

### Flow Cytometry Analysis:

Experimental: Peripheral blood mononuclear cells (PBMCs) were first treated with a solution of CELLBLOX Monocyte and Macrophage Blocking Buffer to prevent non-specific binding of the antibody or nucleic acid nanostructure to the cell surface. Next, the cells were stained with either the monomer or dimer CD4 stains. An unstained cell sample was prepared as well. Finally, the cells were analyzed on a CYTEK Aurora flow cytometer (Cytek Biosciences, Fremont, CA). The FCS files were processed with FLOWJO software.

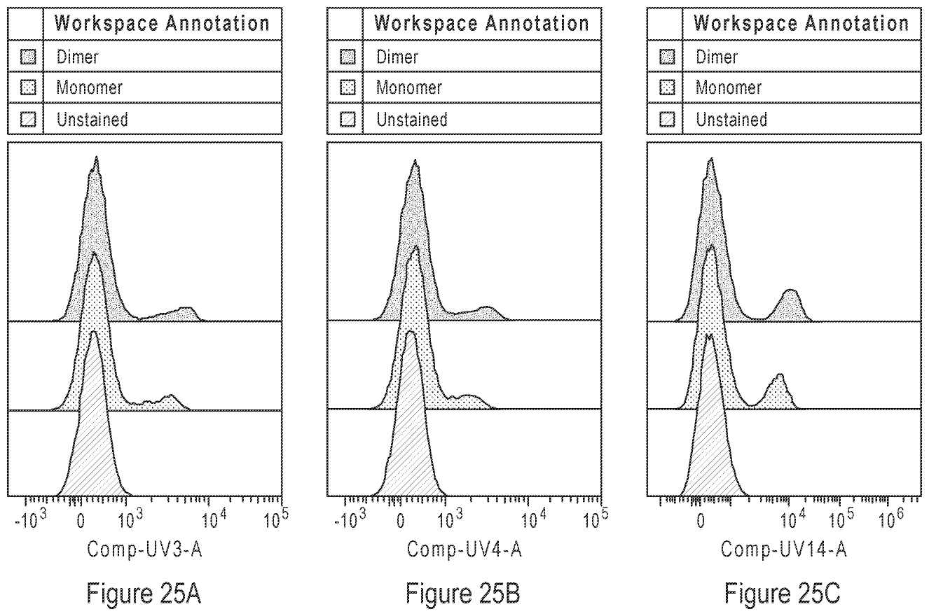

Results: In the flow cytometry experiment we expected to see an amplified signal relative to the monomer control. As seen in FIGS. 25A-C and Table 3 below, the signals from dimer were brighter than the monomer by a factor of 1.3 to 1.7.

TABLE 3
Positive median fluorescence intensity (MFI+) in each labels primary
detector channel from monomer and dimer NOVAFLUOR Ultraviolet
CD4 stained PBMCs. The x Factor indicates signal intensity increase
relative to the monomer. Primary detector channels were UV3, UV4,
and UV14 for NOVAFLUOR Ultraviolet 430 (NF-Ultraviolet 430),
NOVAFLUOR Ultraviolet 445 (NF-Ultraviolet 445), and NOVAFLUOR
Ultraviolet 755 (NF-Ultraviolet 755), respectively.

| Sample Name | Monomer MFI+ | Dimer MFI+ | x Factor, MFI |
|---|---|---|---|
| NF-Ultraviolet 430 | 3,128 | 4,165 | 1.3 |
| NF-Ultraviolet 445 | 1,752 | 2,569 | 1.5 |
| NF-Ultraviolet 755 | 5,849 | 9,739 | 1.7 |

Summary Statement: The flow cytometry data provided evidence the dimers were capable of amplifying the fluorescence signal as described herein. The fact amplification works across multiple types of nucleic acid nanostructure compositions provided further evidence of the efficacy and versatility of this technique to amplify fluorescence signal for any nucleic acid nanostructure.

The breadth and scope of the present disclosure should not be limited by any of the above-described exemplary embodiments but should be defined only in accordance with the following claims and their equivalents.

### REFERENCES

1. “Introduction to Secondary Antibodies” Thermo Fisher Scientific, 9 Sep. 2020, https://www.thermofisher.com/us/en/home/life-science/antibodies/antibodies-learning-center/antibodies-resource-library/antibody-methods/introduction-secondary-antibodies.html.
2. “Anti-Dye Antibodies” Thermo Fisher Scientific, 9 Sep. 2020, https://www.thermofisher.com/us/en/home/life-science/antibodies/primary-antibodies/epitope-tag-antibodies/anti-dye-antibodies.html.
3. Player A N, Shen L P, Kenny D, Antao V P, Kolberg J A. Single-copy gene detection using branched DNA (bDNA) in situ hybridization. J Histochem Cytochem. 2001; 49(5):603-612.
4. “RNA Detection by Flow Cytometry” Thermo Fisher Scientific, 9 Sep. 2020, https://www.thermofisher.com/us/en/home/life-science/cell-analysis/flow-cytometry/flow-cytometry-assays-reagents/rna-detection-flow-cytometry.html.
5. U.S. Patent Publication No. 2020/0124532. Lebeck, A., Dwyer, C., LaBoda C. Resonator Networks for Improved Label Detection, Computation, Analyte Sensing, and Tunable Random Number Generation.

What is claimed is:

1. A nucleic acid nanostructure complex comprising a primary nucleic acid nanostructure linked to one or more secondary nucleic acid nanostructures.

2. The nanostructure complex of claim 1, wherein the primary nanostructure is linked to a specificity determining molecule.

3. The nanostructure complex of claim 1 or 2, wherein (i) the primary nanostructure is linked to the specificity determining molecule and/or secondary nanostructures via a nucleic acid linker or (ii) wherein the primary nanostructure is linked to the specificity determining molecule via a biotin/biotin-binding protein complex and linked to the secondary nanostructures via a nucleic acid linker;
optionally, wherein the nucleic acid linker is a hybridized at least partially double-stranded linker;
optionally, wherein more than one primary nanostructure is linked to the specificity determining molecule via the same biotin/biotin-binding protein complex;
optionally, wherein the biotin-binding protein is avidin, streptavidin, or NeutrAvidin.

4. The nanostructure complex of claim 3, comprising the general structure:
P(-L-S)ₙ
wherein P is the primary nanostructure, L is a hybridized at least partially double-stranded nucleic acid linker, S is one or more adjacently linked secondary nanostructures, and n is an integer greater than zero, and wherein:
(i) the primary nanostructure comprises n number of partially single-stranded nucleic acid linker extensions,
(ii) there are n number of secondary nanostructures comprising an at least partially single-stranded nucleic acid linker extension, and
(iii) at least a portion of the nucleic acid sequence of the single-stranded region of the n number of primary nanostructure nucleic acid linker extensions is at least partially complementary to at least a portion of the nucleic acid sequence of the single-stranded region of one of the secondary nucleic acid linker extensions sufficient to form a hybridized at least partially double-stranded linker between the primary nanostructure and each of the secondary nanostructures, thus linking the primary nanostructure to n number of secondary nanostructures.

5. The nanostructure complex of claim 4, wherein:
(a) the primary nanostructure is adjacently linked to one secondary nanostructure, and wherein:
(i) the primary nanostructure comprises an at least partially single-stranded nucleic acid linker extension,
(ii) the secondary nanostructure comprises an at least partially single-stranded nucleic acid linker extension, and
(iii) at least a portion of the nucleic acid sequence of the single-stranded region of the primary nanostructure nucleic acid linker extension is at least partially complementary to at least a portion of the nucleic acid sequence of the single-stranded region of the secondary nanostructure nucleic acid linker extension sufficient to form a hybridized at least partially double-stranded linker, thus linking the primary nanostructure to the secondary nanostructure;
(b) the primary nanostructure is adjacently linked to two secondary nanostructures, and wherein:
(i) the primary nanostructure comprises two at least partially single-stranded nucleic acid linker extensions comprising the same single-stranded hybridizing region sequence,
(ii) both secondary nanostructures comprise an at least partially single-stranded nucleic acid linker extension comprising the same single-stranded hybridizing region sequence, and
(iii) at least a portion of the nucleic acid sequence of the single-stranded hybridizing region of the primary nanostructure nucleic acid linker extensions is at least partially complementary to at least a portion of the nucleic acid sequence of the single-stranded hybridizing region of the secondary nanostructures nucleic acid linker extensions, sufficient to form a hybridized at least partially double-stranded linker between the primary nanostructure and both secondary nanostructures, thus linking the primary nanostructure to the two secondary nanostructures; or
(c) the primary nanostructure is adjacently linked to three secondary nanostructures, and wherein:
(i) the primary nanostructure comprises three at least partially single-stranded nucleic acid linker extensions comprising the same single-stranded hybridizing region sequence,
(ii) all three secondary nanostructures comprise an at least partially single-stranded nucleic acid linker extension, optionally, comprising the same single-stranded hybridizing region sequence, and
(iii) at least a portion of the nucleic acid sequence of the single-stranded hybridizing region of the primary nanostructure nucleic acid linker extensions is at least partially complementary to at least a portion of the nucleic acid sequence of the single-stranded hybridizing region of the secondary nanostructures nucleic acid linker extensions, sufficient to form a hybridized at least partially double-stranded linker between the primary nanostructure and all three secondary nanostructures, thus linking the primary nanostructure to the three secondary nanostructures.

6. The nanostructure complex of claim 4, wherein:
(a) the primary nanostructure is adjacently linked to two secondary nanostructures, and wherein:
(i) the primary nanostructure comprises two at least partially single-stranded nucleic acid linker extensions, wherein each of the at least partially single-stranded nucleic acid linker extensions comprises a different single-stranded hybridizing region sequence,
(ii) both secondary nanostructures comprise an at least partially single-stranded nucleic acid linker extension, wherein each of the at least partially single-stranded nucleic acid linker extensions comprises a different single-stranded hybridizing region sequence, and
(iii) at least a portion of the nucleic acid sequence of the single-stranded hybridizing region of each of the primary nanostructure nucleic acid linker extensions is at least partially complementary to at least a portion of the nucleic acid sequence of the single-stranded hybridizing region of one of the secondary nanostructures nucleic acid linker extensions, sufficient to form a hybridized at least partially double-stranded linker between the primary nanostructure and both secondary nanostructures, thus linking the primary nanostructure to the two secondary nanostructures; or
(b) the primary nanostructure is adjacently linked to three secondary nanostructures, and wherein:
(i) the primary nanostructure comprises three at least partially single-stranded nucleic acid linker extensions, wherein at least one of the at least partially single-stranded nucleic acid linker extensions comprises a different single-stranded hybridizing region sequence, optionally, wherein each of the at least partially single-stranded nucleic acid linker extensions comprises a different single-stranded hybridizing region sequence,
(ii) all three secondary nanostructures comprise an at least partially single-stranded nucleic acid linker extension, wherein at least one of the partially single-stranded nucleic acid linker extensions comprises a different single-stranded hybridizing region sequence, optionally, wherein each of the partially single-stranded nucleic acid linker extensions comprises a different single-stranded hybridizing region sequence, and
(iii) at least a portion of the nucleic acid sequence of the single-stranded hybridizing region of the each of the primary nanostructure nucleic acid linker extensions is at least partially complementary to at least a portion of the nucleic acid sequence of the single-stranded hybridizing region of one of the secondary nanostructures nucleic acid linker extensions, sufficient to form a hybridized at least partially double-stranded linker between the primary nanostructure and all three secondary nanostructures, thus linking the primary nanostructure to the three secondary nanostructures.

7. The nanostructure complex of any one of claims 1 to 6, wherein the primary nanostructure and/or at least one of the secondary nanostructures comprises one or a combination of fluorophore moieties that give the nanostructure complex a spectral profile.

8. The nanostructure complex of claim 7, wherein the primary nanostructure and at least one of the linked secondary nanostructures in combination give the nanostructure complex a spectral profile.

9. The nanostructure complex of any one of claims 1 to 8, wherein the primary nanostructure and/or at least one of the secondary nanostructures comprises a unique identifying sequence.

10. The nanostructure complex of claim 9, wherein the primary nanostructure and/or at least one of the linked secondary nanostructures comprises two or more unique identifying sequences.

11. The nanostructure complex of claim 9 or 10,
wherein the primary nanostructure comprises a unique identifying sequence and one or a combination of fluorophore moieties that give the primary nanostructure a spectral profile; and/or wherein at least one of the linked secondary nanostructures comprises a unique identifying sequence and one or a combination of fluorophore moieties that give the secondary nanostructure a spectral profile.

12. The nanostructure complex of any one of claims 1 to 11, wherein the primary nanostructure comprises one or a combination of fluorophore moieties that give the primary nanostructure a spectral profile and is linked to n number of secondary nanostructures having the same spectral profile, wherein the secondary nanostructures with the same spectral profile amplify the fluorescence signal of the nanostructure complex in comparison to the fluorescence signal of the primary nanostructure alone;
optionally, wherein the secondary nanostructures having the same spectral profile as the primary nanostructure also each comprise the same number of fluorophore moieties as the primary nanostructure and amplify the fluorescence signal of the nanostructure complex by a factor of about n+0.5, n+0.6, n+0.7, n+0.8, n+0.9 or n+1 times the amount of the fluorescence signal of the primary nanostructure alone.

13. The nanostructure complex of any one of claims 1 to 12, wherein the primary nanostructure comprises a unique identifying sequence and is linked to n number of secondary nanostructures comprising the same unique identifying sequence, wherein the secondary nanostructures with the same unique identifying sequence amplify the sequencing signal of the nanostructure complex in comparison to the sequencing signal of primary nanostructure alone;
optionally, wherein the secondary nanostructures also each comprise the same number of unique identifying sequences as the primary nanostructure and amplify the sequencing signal of the nanostructure complex by a factor of about n+0.5, n+0.6, n+0.7, n+0.8, n+0.9 or n+1 times the amount of the sequencing signal of the primary nanostructure alone.

14. The nanostructure complex of any one of claims 1 to 13, wherein the primary nanostructure comprises one or a combination of fluorophore moieties and is linked to a secondary nanostructure comprising one or more quenchers that absorb the fluorescence of and/or undergo Forster resonance energy transfer with the fluorophore moieties of the primary nanostructure.

15. The nanostructure complex of any one of claims 1 to 14, wherein the primary nanostructure comprises one or a combination of fluorophore moieties that give the primary nanostructure a spectral profile and is linked to a secondary nanostructure comprising a different fluorophore moiety or different combination of fluorophore moieties that give the secondary nanostructure and/or the nanostructure complex a spectral profile that is different from the primary nanostructure spectral profile.

16. The nanostructure complex of any one of claims 1 to 15,
wherein the primary nanostructure comprises x number of a fluorophore moiety or a combination of fluorophore moieties and is linked to y number of secondary nanostructures each comprising z number of the same fluorophore moiety or combination of fluorophore moieties as the primary nanostructure, wherein z can be independently determined for each secondary nanostructure and the sum of z from the secondary nanostructures is zₜₒₜₐₗ;
wherein the fluorescent signal of the nanostructure complex compared to the fluorescent signal of the primary nanostructure alone is amplified by a factor of about (x+zₜₒₜₐₗ)/x;
optionally, wherein z is the same for each secondary nanostructure and zₜₒₜₐₗ=y*z.

17. The nanostructure complex of any one of claims 1 to 16, comprising a primary nucleic acid nanostructure adjacently linked to one or more proximal secondary nucleic acid nanostructures, wherein at least one of the proximal secondary nanostructure is further linked to another secondary nanostructure, optionally, wherein the primary nanostructure and the one or more proximal secondary nanostructures and/or the one or more proximal secondary nanostructures and the another secondary nanostructure linked to the proximal secondary nanostructure is linked via a hybridized at least partially double-stranded nucleic acid linker.

18. The nanostructure complex of claim 17 comprising the general formula:
P-L₁-S_{P}-L₂-(Sₓ-L_{y})ₙ-Z
wherein P is the primary nanostructure, L₁ is a linker linking the primary nanostructure to a secondary nanostructure, S_{P} (proximal secondary nanostructure) is a secondary nanostructure adjacently linked to the primary nanostructure, L₂ is a linker linking S_{P} to another secondary nanostructure, n is zero or a positive integer, (Sₓ-L_{y}) comprises a secondary nanostructure Sₓ and linker L_{y} linking Sₓ to an additional secondary nanostructure, and Z is an additional one or more secondary nanostructures;
optionally, wherein Z is a terminal secondary nanostructure S_{T}.

19. The nanostructure complex of claim 17 or 18, wherein L₁, L₂, and/or one or more of L_{y} are a hybridized at least partially double-stranded nucleic acid linker and wherein:
L₁ and L₂ comprise the same hybridized region sequence;
L₁ and L₂ comprise different hybridized region sequences;
L₁ and L_{y} comprise the same hybridized region sequence;
L₁ and L_{y} comprise different hybridized region sequences;
L₂ and L_{y} comprise the same hybridized region sequence;
L₂ and L_{y} comprise different hybridized region sequences;
L₁, L₂, and L_{y} all comprise the same hybridized region sequence; and/or
L₁, L₂, and L_{y} each comprise different hybridized region sequences.

20. The nanostructure complex of any one of claims 17 to 19,
wherein n is zero and the nanostructure complex comprises the general formula:
P-L₁-S_{P}-L₂-Z
optionally, wherein Z is S_{T} (P-L₁-S_{P}-L₂-S_{T});
wherein n is one and the nanostructure complex comprises the general formula:
P-L₁-S_{P}-L₂-Sₓ-L_{y}-Z
optionally, wherein Z is S_{T} (P-L₁-S_{P}-L₂-Sₓ-L_{y}-S_{T});
wherein n is two and the nanostructure complex comprises the general formula:
P-L₁-S_{P}-L₂-S_{x[a]}-L_{y[a]}-S_{x[b]}-L_{y[c]}-Z
optionally, wherein Z is S_{T} (P-L₁-S_{P}-L₂-S_{x[a]}-L_{y[a]}-S_{x[b]}-L_{y[b]}-S_{T}); or
wherein n is three and the nanostructure complex comprises the general formula:
P-L₁-S_{P}-L₂-S_{x[a]}-L_{y[a]}-S_{x[b]}-L_{y[c]}-S_{x[c]}-L_{y[c]}-Z
optionally, wherein Z is S_{T} (P-L₁-S_{P}-L₂-S_{x[a]}-L_{y[a]}-S_{x[b]}-L_{y[b]}-S_{x[c]}-L_{y[c]}-S_{T}).

21. The nanostructure complex of any one of claims 17 to 20, wherein L₁, L₂, and L_{y} are hybridized at least partially double-stranded nucleic acid linkers each comprising a different hybridized region sequence and wherein n is two or more and at least two L_{y} comprise the same hybridized region sequence.

22. The nanostructure complex of any one of claims 17 to 20, wherein L₁, L₂, and L_{y} are hybridized at least partially double-stranded nucleic acid linkers each comprising a different hybridized region sequence and wherein n is two or more and all L_{y} comprise the same hybridized region sequence.

23. The nanostructure complex of any one of claims 17 to 20, wherein L₁, L₂, and L_{y} are hybridized at least partially double-stranded nucleic acid linkers each comprising a different hybridized region sequence and wherein n is two or more and all L_{y}, except for the L_{y} linking Sₓ to S_{T}, comprise the same hybridized region sequence.

24. The nanostructure complex of any one of claims 17 to 23, wherein the identity and/or order of secondary nanostructures attached to a primary nanostructure is determined by the hybridizing region sequences of the at least partially single-stranded linker extensions of the secondary nanostructures.

25. The nanostructure complex of any one of claims 17 to 24, wherein a terminal secondary nanostructure can be linked by hybridization to no more than one primary or secondary nanostructure;
optionally, wherein the terminal secondary nanostructure only comprises one at least partially single-stranded linker extension for hybridization to another secondary nanostructure.

26. The nanostructure complex of any one of claims 17 to 25, wherein at least one secondary nanostructure comprises one or more quenchers that absorb the fluorescence of and/or undergo Forster resonance energy transfer with the fluorophore moieties of another nanostructure;
optionally, wherein a terminal secondary nanostructure comprises one or more quenchers that absorb the fluorescence of and/or undergo Forster resonance energy transfer with the fluorophore moieties of another nanostructure.

27. The nanostructure complex of any one of claims 17 to 26, wherein at least one proximal secondary nanostructure is adjacently linked to two or more other secondary nanostructures.

28. The nanostructure complex of any one of claims 17 to 27, wherein at least one non-proximal secondary nanostructure is adjacently linked to three or more other secondary nanostructures.

29. A nanostructure complex comprising a nucleic acid scaffold to which is attached at least one primary nucleic acid nanostructure, wherein the primary nanostructure comprises an at least partially single-stranded nucleic acid linker extension that is hybridized to at least a portion of sequence of the nucleic acid scaffold.

30. The nanostructure complex of claim 29, wherein the nucleic acid scaffold is linked to a specificity determining molecule;
optionally, wherein the complex is bound to a target via the specificity determining molecule.

31. The nanostructure complex of claim 29 or 30, wherein at least two primary nanostructures are attached to the nucleic acid scaffold via hybridization.

32. The nanostructure complex of any one of claims 29 to 31, wherein at least one primary nanostructure is linked to one or more secondary nanostructures.

33. The nanostructure complex of any one of claims 29 to 32, wherein at least one nanostructure of the complex is a fluorescent nanostructure and/or comprises a unique identifying sequence.

34. A multiplexed composition comprising two or more different primary nanostructures, at least one of which is part of a nanostructure complex according to any one of claims 1 to 33.

35. The multiplexed composition of claim 34,
wherein at least one primary nanostructure comprises one or a combination of fluorophore moieties that give the primary nanostructure a spectral profile and the different primary nanostructures differ at least in the presence of fluorophore moieties, number of fluorophore moieties, position of the fluorophore moieties, and/or spectral profile of the fluorophore moieties; and/or
wherein at least one primary nanostructure comprises a unique identifying sequence and the different primary nanostructures differ at least in the presence, number, and/or sequence of the unique identifying sequence.

36. The multiplexed composition of claim 34 or 35, wherein at least two of the different primary nanostructures are conjugated to different specificity determining molecules.

37. The multiplexed composition of any one of claims 34 to 36,
wherein at least two of the different primary nanostructures of the composition differ at least by the sequence of one or more nucleic acids comprising the primary nanostructure and/or at least two of the different primary nanostructures of the composition each comprise one or more at least partially single-stranded nucleic acid linker extensions and differ at least by the sequence and/or combination of their one or more at least partially single-stranded nucleic acid linker extensions.

38. The multiplexed composition of any one of claims 34 to 37, comprising at least one primary nanostructure that is not linked to a secondary nanostructure.

39. The multiplexed composition of any one of claims 34 to 38, comprising two or more different nanostructure complexes of any one of claims 1 to 33;
optionally, wherein all of the primary nanostructures are part of a nanostructure complex.

40. The multiplexed composition of claim 39,
wherein at least two of the different nanostructure complexes having different primary nanostructures comprise different secondary nanostructures; and/or
wherein at least two of the different nanostructure complexes having different primary nanostructures comprise the same secondary nanostructure.

41. The multiplexed composition of claim 40,
wherein at least one secondary nanostructure comprises one or more fluorophore moieties that give the secondary nanostructure a spectral profile and the different secondary nanostructures differ at least in the presence of fluorophore moieties, number of fluorophore moieties, position of the fluorophore moieties, and/or spectral profile of the fluorophore moieties; and/or
wherein at least one secondary nanostructure comprises a unique identifying sequence and the different secondary nanostructures differ at least in the presence, number, and/or sequence of the unique identifying sequence.

42. The multiplexed composition of any one of claims 34 to 41,
wherein at least two of the different secondary nanostructures differ at least by the sequence of one or more nucleic acids comprising the secondary nanostructure and/or at least two of the different secondary nanostructures each comprise an at least partially single-stranded nucleic acid linker extension and differ at least by the hybridizing region sequence of their at least partially single-stranded nucleic acid linker extensions;
optionally, wherein the at least two different secondary nanostructures comprise the same spectral profile, sequencing signal, and/or fluorescence or sequence signal intensity.

43. The multiplexed composition of any one of claims 34 to 42, wherein for each nanostructure complex, the primary nanostructure and each of its linked secondary nanostructures comprise the same spectral profile, sequencing signal, and/or fluorescence or sequence signal intensity.

44. The multiplexed composition of any one of claims 34 to 43, wherein in at least one nanostructure complex the primary nanostructure comprises one or a combination of fluorophore moieties and is linked to a secondary nanostructure comprising one or more quenchers that absorb the fluorescence of and/or undergo Förster resonance energy transfer with the fluorophore moieties of the primary nanostructure.

45. The multiplexed composition of any one of claims 34 to 44, comprising at least two nanostructure complexes wherein:
(i) the primary nanostructure of a first nanostructure complex comprises one or a combination of fluorophore moieties that give the primary nanostructure a spectral profile, wherein the primary nanostructure of said first nanostructure complex is linked to a secondary nanostructure comprising one or a combination of fluorophore moieties that give the secondary nanostructure a different spectral profile from its linked primary nanostructure and/or that give the first nanostructure complex a different spectral profile from its primary nanostructure; and
(ii) the primary nanostructure of an at least second nanostructure complex comprises one or a combination of fluorophore moieties that give the primary nanostructure a different spectral profile than the spectral profile of the primary nanostructure of the first nanostructure complex, wherein the primary nanostructure of the at least second nanostructure complex is also linked to a secondary nanostructure comprising one or a combination of fluorophore moieties that give the secondary nanostructure a different spectral profile from its linked primary nanostructure and/or that give the second nanostructure complex a different spectral profile from its primary nanostructure.

46. The multiplexed composition of any one of claims 34 to 45, comprising at least two nanostructure complexes wherein the primary nanostructure of a first nanostructure complex comprises one or a combination of fluorophore moieties that give the primary nanostructure a first spectral profile and the primary nanostructure of an at least second nanostructure complex also comprises one or a combination of fluorophore moieties that give the primary nanostructure a second spectral profile that is different from the first spectral profile of the primary nanostructure of the first nanostructure complex, and
wherein the secondary nanostructure linked to the primary nanostructure of the first nanostructure complex comprises one or a combination of fluorophore moieties that give it a spectral profile and the secondary nanostructure linked to the primary nanostructure of the at least second nanostructure complex comprises the same one or combination of fluorophore moieties as the secondary nanostructure linked to the primary nanostructure of the first nanostructure complex, optionally, wherein the secondary nanostructure linked to the primary nanostructure of the first nanostructure complex and the secondary nanostructure linked to the primary nanostructure of the second nanostructure complex are the same.

47. The multiplexed composition of any one of claims 34 to 46, wherein at least two nanostructure complexes are fluorescent nanostructure complexes and differ from each other at least by their total number of fluorophore moieties and/or by their intensity of fluorescent signal;
optionally, wherein the nanostructure complexes also differ from each other by their spectral profiles.

48. The multiplexed composition of claim 47,
wherein the number of fluorophore moieties of the primary nanostructure of one nanostructure complex differs from the number of fluorophore moieties of another primary nanostructure complex in the composition; and/or
wherein the number of fluorophore moieties of a secondary nanostructure of one nanostructure complex differs from the number of fluorophore moieties of another secondary nanostructure complex in the composition.

49. The multiplexed composition of any one of claims 34 to 48, wherein at least one nanostructure complex comprises a primary nanostructure linked to two or more secondary nanostructures.

50. The multiplexed composition of any one of claims 34 to 49, wherein two or more nanostructure complexes comprise a primary nanostructure linked to two or more secondary nanostructures;
optionally, wherein at least one nanostructure complex comprising a primary nanostructure linked to two or more secondary nanostructures has a different number of secondary nanostructures than at least one other nanostructure complex comprising a primary nanostructure linked to two or more secondary nanostructures.

51. The multiplexed composition of any one of claims 34 to 50, wherein at least two nanostructure complexes comprise a unique identifying sequence and differ from each other at least by their total number of unique identifying sequences and/or by the intensity of their sequencing signal;
optionally, wherein the nanostructure complexes also differ from each other by the sequence of their unique identifying sequences.

52. The multiplexed composition of any one of claims 34 to 51, wherein at least two nanostructure complexes bind to the same target molecule and wherein at least one of said at least two nanostructure complexes comprises a unique identifying sequence and at least one other of said at least two nanostructure complexes does not comprise a unique identifying sequence with the same sequence or does not comprise a unique identifying sequence.

53. The multiplexed composition of claim 51 or 52,
wherein the number of unique identifying sequences of a primary nanostructure of one nanostructure complex differs from the number of unique identifying sequences of another primary nanostructure of another nanostructure complex in the composition; and/or
wherein the number of unique identifying sequences of a secondary nanostructure of one nanostructure complex differs from the number of unique identifying sequences of another secondary nanostructure of another nanostructure complex in the composition.

54. The multiplexed composition of any one of claims 51 to 53, wherein at least one nanostructure complex comprises a primary nanostructure linked to two or more secondary nanostructures.

55. The multiplexed composition of any one of claims 51 to 54, wherein two or more nanostructure complexes comprise a primary nanostructure linked to two or more secondary nanostructures;
optionally, wherein at least one nanostructure complex comprising a primary nanostructure linked to two or more secondary nanostructures has a different number of secondary nanostructures than at least one other nanostructure complex comprising a primary nanostructure linked to two or more secondary nanostructures.

56. The multiplexed composition of any one of claims 34 to 55, comprising at least two nanostructure complexes and wherein at least two of the nanostructure complexes comprise a primary nanostructure linked to one or a combination of secondary nanostructures and further wherein the one or combination of secondary nanostructures of one nanostructure complex is different from the one or combination of secondary nanostructures of another nanostructure in the composition.

57. The multiplexed composition of claim 56,
wherein the one nanostructure complex has a different spectral profile and/or different intensity of fluorescent signal than that of at least one other nanostructure complex in the composition;
wherein one nanostructure complex has a different sequencing signal and/or different intensity of sequencing signal than that of at least one other nanostructure complex in the composition; and/or
wherein the at least one nanostructure complex has a unique fluorescence identity and/or sequencing identity to at least one other nanostructure complex and/or from any other nanostructure complex in the composition.

58. The multiplexed composition of any one of claims 34 to 57, wherein the secondary nanostructure or combination of secondary nanostructures linked to a primary nanostructure is determined by sequence complementarity of their at least partially single-stranded linker extensions with the sequence of the at least partially single-stranded linker extensions of the primary nanostructure.

59. The multiplexed composition of claim 58, where the sequence of the at least partially single-stranded linker extensions of one primary nanostructure is distinct for purposes of hybridization from the sequence of the at least partially single-stranded linker extensions of at least one other primary nanostructure in the composition.

60. The nanostructure complex of any one of claims 1 to 33 or a nanostructure of the multiplexed composition of any one of claims 34 to 59, wherein at least one nanostructure complex is bound to a target.

61. A method of labeling a target molecule, the method comprising binding a nucleic acid nanostructure complex of any one of claims 1 to 33 to the target molecule.

62. The method of claim 61 comprising:
(i) attaching a primary nucleic acid nanostructure to the target molecule, wherein the primary nucleic acid nanostructure specifically binds to the target molecule; and
(ii) attaching one or more secondary nanostructures to the primary nanostructure bound to the target molecule,
thus forming a nanostructure complex bound to the target molecule;
optionally, wherein the one or more secondary nanostructures is attached to the primary nanostructure via hybridization between an at least partially single-stranded linker extension of the secondary nanostructure and an at least partially single-stranded linker extension of the primary nanostructure to form a hybridized at least partially double-stranded nucleic acid linker.

63. The method of claim 61 comprising:
(i) attaching a specificity determining molecule to the target molecule, wherein the specificity determining molecule specifically binds to the target molecule;
(ii) attaching a primary nanostructure to the specificity determining molecule bound to the target molecule; and
(iii) attaching one or more secondary nanostructures to the primary nanostructure bound to the specificity determining molecule,
thus forming a nanostructure complex bound to the target molecule;
optionally,
(a) wherein the primary nanostructure is attached to the specificity determining molecule via hybridization between an at least partially single-stranded linker extension of the primary nanostructure and an at least partially single-stranded linker extension of the specificity determining molecule to form a hybridized at least partially double-stranded nucleic acid linker; or
(b) wherein the primary nanostructure is attached to the specificity determining molecule via a biotin/biotin-binding protein complex; and/or
optionally, wherein the one or more secondary nanostructures is attached to the primary nanostructure via hybridization between an at least partially single-stranded linker extension of the secondary nanostructure and an at least partially single-stranded linker extension of the primary nanostructure to form a hybridized at least partially double-stranded nucleic acid linker.

64. The method of claim 62 or 63 further comprising attaching at least one additional secondary nanostructure to a proximal secondary nanostructure that is already attached to a primary nanostructure;
optionally, wherein the at least one additional secondary nanostructure is attached to the proximal secondary nanostructure via hybridization between an at least partially single-stranded linker extension of the additional secondary nanostructure and an at least partially single-stranded linker extension of the proximal secondary nanostructure to form a hybridized at least partially double-stranded nucleic acid linker.

65. The method of claim 64, further comprising attaching at least one further additional secondary nanostructure to a secondary nanostructure that is already attached to a secondary nanostructure attached to a proximal secondary nanostructure, either adjacently or through intervening secondary nanostructures;
optionally, wherein the at least one further additional secondary nanostructure is attached to another secondary nanostructure via hybridization between an at least partially single-stranded linker extension of the further additional secondary nanostructure and an at least partially single-stranded linker extension of the other secondary nanostructure to form a hybridized at least partially double-stranded nucleic acid linker;
optionally, wherein the addition of the at least one further additional secondary nanostructure forms a linear polymer of secondary nanostructures attached to the primary nanostructure; and/or
optionally, wherein the addition of two or more further additional secondary nanostructures forms a branched network of secondary nanostructures.

66. The method of any one of claim 64 or 65,
wherein two or more secondary nanostructures are linked together before they are incorporated into the nanostructure complex; and/or
wherein two or more secondary nanostructures are simultaneously contacted with a primary nanostructure or a nanostructure complex that has already been partially assembled to comprise a primary nanostructure and at least one proximal secondary nanostructure, and wherein the order of incorporation into the nanostructure complex of the two or more secondary nanostructures is determined by the sequence of an at least partially single-stranded nucleic acid linker extension of the secondary nanostructures.

67. The method of any one of claims 61 to 66, wherein:
(i) the primary nanostructure and the specificity determining molecule comprising the nucleic acid nanostructure complex are already assembled together before binding the nanostructure complex to the target molecule;
(ii) the primary nanostructure and one or more secondary nanostructures comprising the nucleic acid nanostructure complex are already assembled together before binding the nanostructure complex to the target molecule;
(iii) the primary nanostructure and all secondary nanostructures comprising the nucleic acid nanostructure complex are assembled together before binding the nanostructure complex to the target molecule;
(iv) the primary nanostructure and one or more secondary nanostructures comprising the nucleic acid nanostructure complex are assembled together before attaching the primary nanostructure to the specificity determining molecule, after which the nanostructure complex is bound to the target molecule; and/or
(v) the primary nanostructure and all secondary nanostructures comprising the nucleic acid nanostructure complex are assembled together before attaching the primary nanostructure to the specificity determining molecule, after which the nanostructure complex is bound to the target molecule.

68. The method of claim 67, wherein the primary nanostructure and all secondary nanostructures comprising the nanostructure complex are assembled together, except for one or more final terminal secondary nanostructures, before attaching the primary nanostructure to the specificity determining molecule and/or before binding the nucleic acid nanostructure complex to the target molecule, optionally, wherein none of the final terminal secondary nanostructures are assembled onto the nanostructure complex before binding to the target molecule;
optionally, further attaching the final terminal secondary nanostructures following binding the rest of the assembled nanostructure complex to the target molecule.

69. The method of any one of claims 61 to 68, wherein at least the primary nanostructure and/or at least one secondary nanostructure of the nanostructure complex bound to the target molecule is a fluorescent nanostructure and/or comprises a unique identifying sequence.

70. The method of any one of claims 61 to 69, further comprising measuring for a fluorescent signal and/or sequence signal from the labeled target molecule;
optionally, wherein a fluorescent signal and/or sequencing signal is detected.

71. The method of claim 70, comprising measuring for a fluorescent signal and/or sequencing signal after the primary nanostructure and/or nanostructure complex is bound to the target molecule but before the nanostructure complex is completely assembled and then measuring for a fluorescent signal and/or sequencing signal at least one additional time after at least one secondary nanostructure or additional secondary nanostructure is assembled into the nanostructure complex.

72. The method of claim 70 or 71, comprising measuring for a fluorescent signal from the labeled target molecule before a final terminal secondary nanostructure is attached and then measuring for a fluorescent signal after the final terminal secondary nanostructure is attached.

73. A multiplex method of labeling one or more target molecules, the method comprising binding two or more nanostructure complexes of any one of claims 1 to 33 or at least one nanostructure complex of any one of claims 1 to 33 and at least one primary nanostructure to the one or more target molecules according the method of any one of claims 61 to 72.

74. The multiplex method of claim 73, wherein:
(i) at least two of the nanostructure complexes bind to the same target molecule and the nanostructure complexes differ in at least spectral profile, fluorescent intensity, sequencing signal, and/or sequencing signal intensity;
(ii) at least two of the nanostructure complexes bind to different target molecules and each of the nanostructure complexes have the same spectral profile, fluorescent intensity, sequencing signal, and/or sequencing signal intensity; or
(iii) at least two of the nanostructure complexes bind to different target molecules and each of the nanostructure complexes differ in at least spectral profile, fluorescent intensity, sequencing signal, and/or sequencing signal intensity;
optionally, wherein different nanostructure complexes differ at least in their primary nanostructures and/or wherein different nanostructure complexes differ at least in secondary nanostructures and/or number of secondary nanostructures.

75. The multiplex method of claim 73 or 74, wherein at least two different target molecules are bound by the same nanostructure complex or to nanostructure complexes having at least the same spectral profile, fluorescent signal intensity, sequencing signal, and/or sequencing signal intensity, and wherein at least one other target molecule is bound to a nanostructure complex that differs in at least spectral profile, fluorescent signal intensity, sequencing signal, and/or sequencing signal intensity.

76. The multiplex method of claim 73 or 74, wherein each target molecule is bound to a different nanostructure complex that differs from the other nanostructure complex or complexes at least in spectral profile, fluorescent intensity, sequencing signal, and/or sequencing signal intensity.

77. The multiplex method of claim 73, wherein:
(i) at least one nanostructure complex and at least one primary nanostructure bind to the same target molecule; or
(ii) at least one nanostructure complex and at least one primary nanostructure bind to different target molecules.

78. The method of any one of claims 61 to 77, wherein for at least one nanostructure complex, the addition of one or more secondary nanostructures amplifies the fluorescence signal and/or sequencing signal in comparison to the primary nanostructure alone; optionally, wherein the amplification of the signal can be stoichiometrically controlled.

79. The method of any one of claims 61 to 78, wherein for at least one nanostructure complex, the primary nanostructure comprises one or a combination of fluorophore moieties that give the primary nanostructure a spectral profile and is linked to n number of secondary nanostructures having the same spectral profile, wherein the secondary nanostructures with the same spectral profile amplify the fluorescence signal of the nanostructure complex in comparison to the fluorescence signal of the primary nanostructure alone;
optionally, wherein the secondary nanostructures having the same spectral profile as the primary nanostructure also each comprise the same number of fluorophore moieties as the primary nanostructure and amplify the fluorescence signal of the nanostructure complex by a factor of about n+0.5, n+0.6, n+0.7, n+0.8, n+0.9 or n+1 times the amount of the fluorescence signal of the primary nanostructure alone;
optionally, wherein at least two nanostructure complexes are used in a multiplex method.

80. The method of any one of claims 61 to 79, wherein for at least one nanostructure complex, the primary nanostructure comprises a unique identifying sequence and is linked to n number of secondary nanostructures comprising the same unique identifying sequence, wherein the secondary nanostructures with the same unique identifying sequence amplify the sequencing signal of the nanostructure complex in comparison to the sequencing signal of primary nanostructure alone;
optionally, wherein the secondary nanostructures also each comprise the same number of unique identifying sequences as the primary nanostructure and amplify the sequencing signal of the nanostructure complex by a factor of about n+0.5, n+0.6, n+0.7, n+0.8, n+0.9 or n+1 times the amount of the sequencing signal of the primary nanostructure alone;
optionally, wherein at least two nanostructure complexes are used in a multiplex method.

81. The method of any one of claims 61 to 80, wherein for at least one nanostructure complex, the primary nanostructure comprises one or a combination of fluorophore moieties and is linked to a secondary nanostructure comprising one or more quenchers that absorb the fluorescence of and/or undergo Forster resonance energy transfer with the fluorophore moieties of the primary nanostructure;
optionally, wherein at least two nanostructure complexes are used in a multiplex method.

82. The method of any one of claims 61 to 81, wherein for at least one nanostructure complex, the primary nanostructure comprises one or a combination of fluorophore moieties that give the primary nanostructure a spectral profile and is linked to a secondary nanostructure comprising a different fluorophore moiety or different combination of fluorophore moieties that give the secondary nanostructure and/or the nanostructure complex a spectral profile that is different from the primary nanostructure spectral profile;
optionally, wherein the primary nanostructure is linked to at least two secondary nanostructures each comprising a different fluorophore moiety or a different combination of fluorophore moieties that give each secondary nanostructure and/or the nanostructure complex a spectral profile that is different from the primary nanostructure spectral profile,
further optionally, wherein at least one of the linked secondary nanostructures comprises a different fluorophore moiety or different combination of fluorophore moieties that give such secondary nanostructure a spectral profile that is different from another linked secondary nanostructure spectral profile, and
further optionally, wherein each of the linked secondary nanostructures comprises a different fluorophore moiety or different combination of fluorophore moieties that give each secondary nanostructure a spectral profile that is different from any other linked secondary nanostructure spectral profile;
optionally, wherein the spectral profile of the nanostructure complex is different from the spectral profile of the primary nanostructure;
optionally, wherein the secondary nanostructure comprises a fluorophore moiety or combination of fluorophore moieties that can undergo Forster resonance energy transfer with the fluorophore moiety or combination of fluorophore moieties of the primary nanostructure;
optionally, wherein in the method the fluorescence of the labeled target molecule is measured before and after the addition of a secondary nanostructure that gives the nanostructure complex a different spectral profile; and/or
optionally, wherein at least two nanostructure complexes are used in a multiplex method.

83. The method of any one of claims 61 to 83,
wherein for at least one nanostructure complex, the primary nanostructure comprises x number of a fluorophore moiety or a combination of fluorophore moieties and is linked to y number of secondary nanostructures each comprising z number of the same fluorophore moiety or combination of fluorophore moieties as the primary nanostructure, wherein z is independently determined for each secondary nanostructure and the sum of z from the secondary nanostructures is zₜₒₜₐₗ;
wherein the fluorescent signal of the nanostructure complex compared to the fluorescent signal of the primary nanostructure alone is amplified by a factor of about (x+zₜₒₜₐₗ)/x;
optionally, wherein z is the same for each secondary nanostructure and zₜₒₜₐₗ=y*z; and/or
optionally, wherein at least two nanostructure complexes are used in a multiplex method.

84. A method of detecting a target molecule, the method comprising labeling a target molecule according to the method of any one of claims 61 to 83 and measuring for a fluorescent signal and/or sequence signal from the labeled target molecule;
optionally, wherein the method is a multiplex method and the method comprises labeling at least two target molecules according to the method of any one of claims 61 to 83 and measuring for a fluorescent signal and/or sequence signal from the labeled target molecules.

85. A kit for performing the method of any one of claims 61 to 84,
comprising a nanostructure complex of any one of claims 1 to 59, or a component thereof, and/or
comprising reagents and/or apparatus for labeling a target molecule according to the method of any one of claims 61 to 84;
optionally, wherein the kit further comprises instructions either printed and/or on an electronic storage medium, buffers and/or additional reagents, and/or packaging materials.

86. A nanostructure complex of any of claims 1 to 85, wherein a primary nanostructure and/or a secondary nanostructure comprises a sequence that is a target for a nucleic acid binding protein;
optionally, wherein the nanostructure complex is bound with at least one nucleic acid binding protein; and further,
optionally, wherein the number of bound nucleic acid binding proteins is controlled quantitatively.
